(19) United States
(12) Patent Application Publication
(10) Pub. No.: US 2019/0282501 A1
(43) Pub. Date: Sep. 19, 2019

(54) HYDROALCOHOLIC FOAM FORMULATIONS OF NAFTIFINE

(71) Applicant: FOAMIX PHARMACEUTICALS LTD., Rehovot (IL)

(72) Inventors: Yohan HAZOT, Rehovot (IL); Julia Nomi Biso FEIMAN, Rehovot (IL); Dov TAMARKIN, Maccabim (IL); David SCHUZ, Gimzu (IL); Sarah Marissa CALEY, Chapel Hill, NC (US); Bhushan HARDAS, Summerfield, NC (US)

(73) Assignee: FOAMIX PHARMACEUTICALS LTD., Rehovot (IL)

(21) Appl. No.: 16/072,046

(22) PCT Filed: Jan. 25, 2017
(86) PCT No.: PCT/IB2017/000083

§ 371 Date: Jul. 23, 2018

Related U.S. Application Data
(60) Provisional application No. 62/288,952, filed on Jan. 29, 2016.

(51) Int. Cl. A61K 9/12; A61P 31/10; A61K 31/135; A61K 47/10; A61K 47/38; A61K 47/18; A61K 9/00

(52) U.S. Cl. CPC A61K 9/122; A61P 31/10; A61K 9/124; A61K 9/0014; A61K 47/10; A61K 47/38; A61K 47/183; A61K 31/135

(57) ABSTRACT

The present disclosure relates to topical, hydroalcoholic foam formulations of antifungal agents, including allylamines such as naftifine. In some embodiments, the topical foam formulations comprise a carrier and a liquefied or compressed gas propellant, wherein the carrier comprises about 2% to about 4% by weight of naftifine hydrochloride; about 15% to about 35% by weight of a C₁₋₆ alkyl alcohol; about 15% to about 30% by weight of an alkylene glycol; about 1% to about 2% by weight of a polymer; about 0.5% to about 2.0% by weight of a long chain fatty alcohol; a pH adjuster; about 0.01% to about 0.3% by weight of a chelating agent; water; and optionally about 0.5% to about 1.5% by weight of a preservative; wherein the propellant is present in the formation at about 5% to about 15% by weight of the formulation. Such compositions are useful for treating fungal infections including tinea capitis.

This application is a § 371 national phase entry of International Patent Application No. PCT/IB2017/000083, filed Jan. 25, 2017, which claims priority to U.S. Provisional Application Ser. No. 62/288,952, filed Jan. 29, 2016, all of which are incorporated herein by reference in their entirety.

### TECHNICAL FIELD

The present disclosure relates to topical, hydroalcoholic foam formulations of antifungal agents, including allylamine drugs such as naftifine. Such compositions are useful for treating fungal infections including tinea capitis.

### BACKGROUND

Tinea capitis is a fungal infection of the scalp and is the most common pediatric tinea infection, but can occur at any age. The condition may affect small areas of the scalp or the entire scalp. Infected areas may become bald, with small black dots where hair has broken off. Some infected areas become inflamed or swollen and can lead to pus-filled sores that may develop into lasting scars. Current therapies require primarily oral antifungal treatments that have many negative side-effects and contraindications. These disadvantages are further magnified in the target patient population for this disease, which is mostly pediatric patients.

### SUMMARY

The present application provides, inter alia, a topical foamable formulation comprising a carrier and a liquefied or compressed gas propellant, wherein the carrier comprises:

about 2% to about 4% by weight of the carrier of naftifine, or a pharmaceutically acceptable salt thereof;

about 15% to about 35% by weight of the carrier of a C₁₋₆ alkyl alcohol;

about 15% to about 30% by weight of the carrier of an alkylene glycol;

about 1% to about 2% by weight of the carrier of a polymer;

about 0.5% to about 2.0% by weight of the carrier of a long chain fatty alcohol;

a pH adjuster;

about 0.01% to about 0.3% by weight of the carrier of a chelating agent; and

water; and

wherein the propellant is present at about 5% to about 15% by weight of the formulation.

In some embodiments, the naftifine, or a pharmaceutically acceptable salt thereof, is naftifine hydrochloride.

In some embodiments, the topical foamable formulation comprises:

about 2.5% to about 3% by weight of the carrier of naftifine hydrochloride;

about 18% to about 22% by weight of the carrier of a C₁₋₆ alkyl alcohol;

about 17% to about 23% by weight of the carrier of an alkylene glycol;

about 1.5% to about 2% by weight of the carrier of a polymer;

about 0.5% to about 1.5% by weight of the carrier of a long chain fatty alcohol;

a pH adjuster;

about 0.01% to about 0.3% by weight of the carrier of a chelating agent; and

water; and

about 5% to about 10% by weight of the formulation of a liquefied or compressed gas propellant.In some embodiments:

the alkylene glycol is propylene glycol; and/or

the polymer is a hydroxyalkyl cellulose; and/or

the pH adjuster is an amine base.

In some embodiments:

the alkylene glycol is propylene glycol in an amount of about 19% by weight of the carrier; and/or

the polymer is a hydroxyalkyl cellulose in an amount of about 1.75% by weight of the carrier; and/or

the pH adjuster is an amine base.

In some embodiments:

the C₁₋₆ alkyl alcohol is ethanol; and/or

the long chain fatty alcohol is cetostearyl alcohol.

In some embodiments:

the C₁₋₆ alkyl alcohol is ethanol in an amount of about 20% by weight of the carrier; and/or

the long chain fatty alcohol is cetostearyl alcohol in an amount of about 1.0% by weight of the carrier.

In some embodiments, the topical foamable formulation comprises:

about 2.5% to about 3% by weight of the carrier of naftifine hydrochloride;

about 28% to about 32% by weight of the carrier of a C₁₋₆ alkyl alcohol;

about 25% to about 30% by weight of the carrier of an alkylene glycol;

about 1.5% to about 2% by weight of the carrier of a polymer;

about 1% to about 2% by weight of the carrier of a long chain fatty alcohol;

a pH adjuster;

about 0.01% to about 0.3% by weight of the carrier of a chelating agent; and

water; and/or

about 5% to about 10% by weight of the formulation of a liquefied or compressed gas propellant.

In some embodiments:

the alkylene glycol is propylene glycol; and/or

the polymer is a hydroxyalkyl cellulose; and/or

the pH adjuster is an amine base.

In some embodiments:

the alkylene glycol is propylene glycol in an amount of about 28.5% by weight of the carrier; and/or

the polymer is a hydroxyalkyl cellulose in an amount of about 1.75% by weight of the carrier; and/or

the pH adjuster is an amine base.

In some embodiments:

the C₁₋₆ alkyl alcohol is ethanol; and/or

the long chain fatty alcohol is cetostearyl alcohol.

In some embodiments:

the C₁₋₆ alkyl alcohol is ethanol in an amount of about 30% by weight of the carrier; and/or

the long chain fatty alcohol is cetostearyl alcohol in an amount of about 1.5% by weight of the carrier.

In some embodiments:

the hydroxyalkyl cellulose is hydroxypropyl cellulose; and/or

the pH adjuster is trolamine; and/or

the chelating agent is edetate disodium.

In some embodiments, the topical foamable formulation further comprises about 0.5% to about 1.5% by weight of the carrier of a preservative.

In some embodiments, the topical foamable formulation further comprises about 1% by weight of the carrier of a preservative.

In some embodiments, the preservative is benzyl alcohol.

In some embodiments, the topical foamable formulation comprises:

a) about 3% by weight of the carrier of naftifine hydrochloride;

b) about 20% by weight of the carrier of ethanol;

c) about 19% by weight of the carrier of propylene glycol;

d) about 1.75% by weight of the carrier of hydroxypropyl cellulose;

e) about 1% by weight of the carrier of cetostearyl alcohol;

f) about 1% by weight of the carrier of benzyl alcohol;

g) about 0.17% by weight of the carrier of trolamine;

h) about 0.02% by weight of the carrier of disodium edetate; and

i) about 54.06% by weight of the carrier of water; and

wherein the propellant is AP-70 propellant present at about 8% by weight of the formulation.

In some embodiments, the topical foamable formulation comprises:

a) about 3% by weight of the carrier of naftifine hydrochloride;

b) about 20% by weight of the carrier of ethanol;

c) about 19% by weight of the carrier of propylene glycol;

d) about 1.75% by weight of the carrier of hydroxypropyl cellulose;

e) about 1% by weight of the carrier of cetostearyl alcohol;

f) about 0.17% by weight of the carrier of trolamine;

g) about 0.02% by weight of the carrier of disodium edetate; and

h) about 55.06% by weight of the carrier of water; and

wherein the propellant is AP-70 propellant at about 8% by weight of the formulation.

In some embodiments, the topical foamable formulation comprises:

a) about 2.5% by weight of the carrier of naftifine hydrochloride;

b) about 20% by weight of the carrier of ethanol;

c) about 19% by weight of the carrier of propylene glycol;

d) about 1.75% by weight of the carrier of hydroxypropyl cellulose;

e) about 1% by weight of the carrier of cetostearyl alcohol;

f) about 1% by weight of the carrier of benzyl alcohol;

g) about 0.14% by weight of the carrier of trolamine;

h) about 0.02% by weight of the carrier of disodium edetate; and

i) about 54.59% by weight of the carrier of water; and

wherein the propellant is AP-70 propellant at about 8% by weight of the formulation.

In some embodiments, the topical foamable formulation comprises:

a) about 2.5% by weight of the carrier of naftifine hydrochloride;

b) about 30% by weight of the carrier of ethanol;

c) about 28.5% by weight of the carrier of propylene glycol;

d) about 1.75% by weight of the carrier of hydroxypropyl cellulose;

e) about 1.5% by weight of the carrier of cetostearyl alcohol;

f) about 1% by weight of the carrier of benzyl alcohol;

g) about 0.14% by weight of the carrier of trolamine;

h) about 0.02% by weight of the carrier of disodium edetate; and

i) about 34.59% by weight of the carrier of water; and

wherein the propellant is AP-70 propellant at about 8% by weight of the formulation.

In some embodiments, the topical foamable formulation comprises:

a) about 3% by weight of the carrier of naftifine hydrochloride;

b) about 30% by weight of the carrier of ethanol;

c) about 28.5% by weight of the carrier of propylene glycol;

d) about 1.75% by weight of the carrier of hydroxypropyl cellulose;

e) about 1.5% by weight of the carrier of cetostearyl alcohol;

f) about 1% by weight of the carrier of benzyl alcohol;

g) about 0.17% by weight of the carrier of trolamine;

h) about 0.02% by weight of the carrier of disodium edetate; and

i) about 34.06% by weight of the carrier of water; and

wherein the propellant is AP-70 propellant at about 8% by weight of the formulation.

In some embodiments, a topical foam or foamable formulation comprises a carrier and a liquefied or compressed gas propellant, wherein the carrier comprises:

about 15% to about 35% by weight of the carrier of a C₁₋₆ alkyl alcohol;

about 15% to about 30% by weight of the carrier of an alkylene glycol;

about 1% to about 2% by weight of the carrier of a polymer;

about 0.5% to about 2.0% by weight of the carrier of a long chain fatty alcohol;

a pH adjuster;

about 0.01% to about 0.3% by weight of the carrier of a chelating agent; and

water; and

wherein the propellant is present at about 5% to about 15% by weight of the formulation.

In some embodiments, a topical foam or foamable formulation comprises a carrier and a liquefied or compressed gas propellant, wherein the carrier comprises:

about 2% to about 4% by weight of the carrier of an anti-fungal drug or a pharmaceutically acceptable salt thereof;

about 15% to about 35% by weight of the carrier of a C1_6 alkyl alcohol;

about 15% to about 30% by weight of the carrier of an alkylene glycol;

about 1% to about 2% by weight of the carrier of a polymer;

about 0.5% to about 2.0% by weight of the carrier of a long chain fatty alcohol;

a pH adjuster;

about 0.01% to about 0.3% by weight of the carrier of a chelating agent; and

water; and

wherein the propellant is present at about 5% to about 15% by weight of the formulation.

According to some embodiments, the anti-fungal drug comprises a polyene, an azole, an allylamine, a benzyl amine, or a pharmaceutically acceptable salt thereof. According to some embodiments, the anti-fungal drug comprises terbinafine, butenafine, amorolfine, nystatin, Amphotericin B, miconazole, clotrimazole, ketoconazole, oxiconazole, eberconazole, econazole, sulconazole, sertaconazle bifonazole, butoconazole, fenticonazole, isoconazole, omoconazole, tioconazole, ciclopirox, selenium sulfide, flucytosine, griseofulvin, candicidin, filipin, hamycin, natamycin, rimocidin, albaconazole, fluconazole, isavuconazole, itraconazole, posaconazole, ravuconazole, terconazole, voriconazole, abafungin, anidulafungin, caspofungin, micafungin, benzoic acid, haloprogin, polygodial, tolnaftate, undecylenic acid, Crystal violet, or a pharmaceutically acceptable salt thereof.

According to some embodiments, the herein disclosed formulations further comprise another active agent. According to some embodiments, the other active agent comprises one or more agents selected from the group consisting of a non-steroidal anti-inflammatory agent, a local anesthetic agent, a steroid, a keratolytically active agent, a retinoid, antipsoriasis agent, an antiinfective agent, an azole, a steroid, an antimicrobial agent, an antibiotic, an allergen, an analgesic agent, an antiallergic agent, an antibacterial agent, an antifungal agent, an antihistamine, an antimycotic agent, an antiparasitic agent, an antipruritic agent, an antiviral agent, a calcium channel blocker, such as flunarizine, a fungicide, an insecticide, a vasoconstrictor, a vasodilator, a vitamin, a vitamin B, a vitamin B derivative, a vitamin C, a vitamin C derivative, a vitamin F, a vitamin F derivative, a vitamin K, and a vitamin K derivative.

According to some embodiments, the herein disclosed topical foam or foamable formulation is free or substantially free of a surfactant.

In some embodiments, the present application further provides methods of treating one or more fungal infections of the skin, particularly of the scalp, and/or of the eyebrows, eyelashes, hair, and/or nails. In some embodiments, the method comprises applying a foam or foamable composition disclosed herein to and/or near the impacted skin. In some embodiments, the method comprises applying a foamable composition disclosed herein to and/or near the impacted skin. In some embodiments, the method comprises applying a foam composition prepared from a foamable composition disclosed herein to and/or near the impacted skin. In some embodiments, described herein is a use of the topical foam or foamable formulation as provided herein in the manufacture of a medicament for treating a fungal infection in a patient in need thereof. In some embodiments, described herein is the topical foam or foamable formulation as disclosed herein for use in the treatment of a fungal infection in a patient in need thereof. In some embodiments, a foam or foamable composition disclosed herein is administered to a patient suffering from a dermatophytosis. In some embodiments, the dermatophytosis is tinea capitis, tinea favosa (infection by Trichophyton schoenleinii), tinea corporis (ringworm of glabrous skin), tinea imbricata (infection by Trichophyton concentiricum), tinea cruris (ringworm of the groin) tinea unguium, or onychomycosis (ringworm of the nail), tinea pedis (ringworm of the feet), tinea barbae (ringworm of the beard), and tinea manuum (ringworm of the hand). In some embodiments, a foam or foamable composition disclosed herein is administered to a patient suffering from tinea capitis.

In some embodiments, a method is disclosed of treating tinea capitis in a patient in need thereof, the method comprising administering to the patient a topical foam formulation prepared from a topical foamable formulation as provided herein.

In some embodiments, described herein is a use of the topical foam or foamable formulation as provided herein in the manufacture of a medicament for treating tinea capitis. In some embodiments, described herein is the topical foam or a foamable formulation as disclosed herein for use in the treatment of tinea capitis.

In some embodiments, the tinea capitis is associated with Trichophyton mentagrophytes. In some embodiments, the tinea capitis is associated with Microsporum canis. In some embodiments, the tinea capitis is associated with Trichophyton tonsurans.

In some embodiments, the topical foam or foamable formulation is administered one, two, or three times per day, or every other day or every three days. In some embodiments, the topical foam or foamable formulation is administered one time per day. In some embodiments, the topical foam or foamable formulation is administered two times per day. In some embodiments, the topical foam or foamable formulation is administered three times per day. In some embodiments, the topical foam or foamable formulation is applied for at least 2, 3, 4, 5, 6, or 7 consecutive days. In some embodiments, the topical foam or foamable formulation is applied for at least 7 consecutive days.

In some embodiments, the present application further provides a method of treating skin, hair, nail, or scalp of a subject infected by a fungal infection comprising administering topically to an infected area on the subject a foamable formulation or foam formulation prepared from a topical foamable formulation as provided herein, wherein the percentage of naftifine released from the formulation is selected from the group consisting of:

at least about 75%, at least about 80%, at least about 85%, and at least about 90% naftifine within 30 minutes of application; and

at least about 95%, at least about 97%, at least about 98%, at least about 99%, and at least about 100% of naftifine within 3 hours of application.

In some embodiments, the percentage of naftifine released from a foam or foamable formulation disclosed herein within 30 minutes of application is about 1-10 times higher (i.e., 1, 1.5, 2, 2.5, 3, 3.5, 4, 4.5, 5, 5.5, 6, 6.5, 7, 7.5, 8, 8.5, 9, 9.5, or 10 times higher, or any factor in between) than a comparable gel or cream formulation and/or about 1-5 times higher (1, 1.1, 1.2, 1.3, 1.4, 1.5, 2, 2.5, 3, 3.5, 4, 4.5, or 5 times higher or any factor in between) within 3 hours of application than a comparable gel or cream formulation. In some embodiments, the percentage of released naftifine is about 9 times higher within 30 minutes of application and/or about 3.3 times higher within 3 hours of application than a comparable cream formulation. In some embodiments, the percentage of naftifine released from the foam or foamable formulation is about 1.5 times higher within 30 minutes of application and/or about 1.2 times higher within 3 hours of application than a comparable gel formulation. In some embodiments, the percentage is calculated as the amount of naftifine released relative to the initial amount of naftifine in the foam or foamable formulation.

In some embodiments, the release rate of naftifine from the foam or foamable formulation provided herein is higher than a comparable gel or cream formulation. In some embodiments, the release of naftifine from the foam or foamable formulation peaks earlier than a comparable gel or cream formulation. As used herein, a formulation “peaks earlier” when a maximal amount of active agent is released earlier from the formulation as compared to other formulations.

In some embodiments, the maximal release of naftifine from the foam or foamable formulation peaks at most about 60 minutes after the initial administration (e.g., about 10, 20, 30, 40, 50, or 60 minutes, or any time period in between). In some embodiments, the maximal release of naftifine from the foam or foamable formulation peaks at about 30 minutes after the initial administration. In some embodiments, the release rate of naftifine from a foam or foamable formulation disclosed herein is about 1-10 times higher (i.e., 1, 1.2, 1.5, 2, 2.5, 3, 3.5, 4, 4.5, 5, 5.5, 6, 6.5, 7, 7.5, 8, 8.5, 9, 9.5, or 10 times higher, or any factor in between) within 30 minutes of application and/or about 1-5 times higher (1, 1.1, 1.2, 1.3, 1.4, 1.5, 2, 2.5, 3, 3.5, 4, 4.5, or 5 times higher or any factor in between) within 3 hours of application than a comparable cream formulation.

In some embodiments, the foam or foamable formulation provided herein shows significant clinical efficacy compared to placebo treatment. In some embodiments, the foam or foamable formulation provided herein shows significant mycological efficacy compared to placebo treatment. In some embodiments, the foam or foamable formulation provided herein shows significant clinical efficacy compared to an untreated group. In some embodiments, the foam or foamable formulation provided herein shows significant mycological efficacy compared to an untreated group. In some embodiments, the foam or foamable formulation provided herein shows significant clinical efficacy compared to placebo treatment. In some embodiments, the foam or foamable composition described herein is also capable of reducing one or more of erythema, desquamation/scaling, and papule scores of skin lesions.

In some embodiments, the effectiveness of the topical foam is assessed by comparing a clinical score of a subject treated with the topical foam to that of a subject treated with a placebo treatment or given no treatment. In some embodiments, the effectiveness of the topical foam is assessed by comparing a number of mycologically affected hairs obtained from a subject treated with the topical foam and that of mycologically affected hairs obtained from a subject treated with a placebo or given no treatment.

In some embodiments, the topical foam reduces the clinical score and/or number of mycologically affected hairs by at least about 25%, at least about 50%, at least about 75%, at least about 90%, or at least about 95% as compared to the placebo or no treatment.

In some embodiments, the topical foamable composition is also capable of reducing one or more of erythema, desquamation/scaling, and papule scores of skin lesions as compared to a placebo treatment or no treatment.

In some embodiments, the topical foam is applied at a dosage sufficient to reduce one or more of erythema, desquamation/scaling, and papule scores of skin lesions as compared to a placebo treatment or no treatment.

In some embodiments, the reduction of one or more of erythema, desquamation/scaling, and papule scores of skin lesions is assessed by a 4-grade assessment scale. In some embodiments, the topical foam reduces one or more of erythema, desquamation/scaling, and papule scores by at least one or more grades as compared to a placebo treatment or no treatment when assessed by a 4-grade assessment scale. In some embodiments, the topical foam results in obtaining a score of 0 or 1 on one or more of erythema, desquamation/scaling, and papule scores of skin lesions at the end of therapy or one week thereafter, as evaluated by a 4-grade assessment scale. In some embodiments, the topical foam is applied at a dosage sufficient to achieve a score of 0 or 1 on one or more of erythema, desquamation/scaling, and papule scores of skin lesions at the end of therapy or one week thereafter, as evaluated by a 4-grade clinical scoring assessment scale. In some embodiments, the topical foam reduces dermatophyte culture obtained from a target skin lesion at the end of therapy or up to about one week thereafter, as compared to an untreated control and/or placebo treatment. In some embodiments, the topical foam results in a negative potassium hydroxide (KOH) test obtained from a target skin lesion at the end of therapy or up to about one week thereafter.

Unless otherwise defined, all technical and scientific terms used herein have the same meaning as commonly understood by one of ordinary skill in the art to which this invention belongs. All ranges disclosed herein include the endpoints. The use of the term “or” shall be construed to mean “and/or” unless the specific context indicates otherwise. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 shows the transported amount of naftifine hydrochloride from intact and collapsed foam formulations B and D as described in Example 4.

FIG. 2A shows a Higuchi plot of the cumulative transport after 3 hours of naftifine hydrochloride from Naftin Cream 2%, Naftin Gel 2%, and naftifine hydrochloride foam formulations B and D as described in Example 4. The values shown are the mean values from the experiment in six-fold.

FIG. 2B shows a Higuchi plot of the percent amount released of naftifine hydrochloride from Naftin Cream 2%, Naftin Gel 2%, and naftifine hydrochloride foam formulations B and D as described in Example 4.

FIG. 3 shows the transported amount of naftifine hydrochloride after 3 hours from Naftin Cream 2%, Naftin Gel 2%, and naftifine hydrochloride foam formulations B and D as described in Example 4. The values shown are the mean values from the experiment conducted in six-fold.

FIG. 4 shows a comparison of the released API after 30 minutes (left bars) and 3 hours (right bars), and the release rate for all tested formulations (Naftin Cream 2%, Naftin Gel 2%, and foam formulations B and D as described in Example 4). The percent released API are based on the applied amount API. The arithmetic mean (n=6) and standard deviation are shown.

FIG. 5 shows the mycological efficacy of each test compound (foam formulation E as described in Example 4) as compared to the untreated control for the topical treatment of Trichophyton mentagrophytes-dermatophytosis in a guinea pig model.

FIG. 6 shows the clinical efficacy of each test compound (foam formulation E as described in Example 4) as compared to the untreated control for the topical treatment of Trichophyton mentagrophytes-dermatophytosis in a guinea pig model.

FIG. 7 shows a comparison of the clinical appearance of the infection site for treated and untreated controls in a Trichophyton mentagrophytes-dermatophytosis guinea pig model.

FIG. 8 shows a mycological evaluation of Trichophyton tonsurans inocula in a Guinea Pig Model.

FIG. 9 shows the mycological efficacy of each test compound (foam formulation E as described in Example 4) as compared to an untreated control for the topical treatment of Trichophyton tonsurans-dermatophytosis in a guinea pig model.

FIG. 10 shows a clinical assessment of Trichophyton tonsurans inocula in a Guinea Pig Model.

FIG. 11 shows a comparison of the clinical appearance of the infection site for Trichophyton tonsurans inocula in a Guinea Pig Model.

FIG. 12 shows the clinical efficacy of each test compound (foam formulations E and F as described in Example 4) as compared to the untreated control for the topical treatment of Trichophyton tonsurans-dermatophytosis in a guinea pig model.

FIG. 13 shows a comparison of the clinical appearance of the infection site for treated and untreated controls in a Trichophyton tonsurans-dermatophytosis guinea pig model.

FIG. 14 shows a mycological evaluation of Microsporum canis inocula in a Guinea Pig Model.

FIG. 15 shows the mycological efficacy of each test compound (foam formulation E as described in Example 4) as compared to an untreated control for the topical treatment of Microsporum canis-dermatophytosis in a guinea pig model.

FIG. 16 shows a clinical assessment of Microsporum canis inocula in a Guinea Pig Model.

FIG. 17 shows a comparison of the clinical appearance of the infection site for Microsporum canis inocula in a Guinea Pig Model.

FIG. 18 shows the clinical efficacy of each test compound (foam formulation E as described in Example 4) as compared to the untreated control for the topical treatment of Microsporum canis-dermatophytosis in a guinea pig model.

FIG. 19 shows a comparison of the clinical appearance of the infection site for treated and untreated controls in a Microsporum canis-dermatophytosis guinea pig model.

### DETAILED DESCRIPTION

Current treatments for tinea capitis are primarily systemic, with griseofulvin being the most prescribed treatment followed by terbinafine. Topical treatment with a naftifine foam that demonstrates comparable efficacy and improved overall safety profile over available oral treatments would differentiate it from current treatments. Accordingly, the present application provides hydroalcoholic foam or foamable formulations of naftifine useful in the treatment of tinea capitis.

### Compositions and Pharmaceutical Formulations

Provided herein are topical foamable formulations comprising a carrier and a liquefied or compressed gas propellant, wherein the carrier comprises:

about 2% to about 4% by weight of the carrier of naftifine hydrochloride;

about 15% to about 35% by weight of the carrier of a C₁₋₆ alkyl alcohol;

about 15% to about 30% by weight of the carrier of an alkylene glycol;

about 1% to about 2% by weight of the carrier of a polymer;

about 0.5% to about 2.0% by weight of the carrier of a long chain fatty alcohol;

a pH adjuster;

about 0.01% to about 0.3% by weight of the carrier of a chelating agent; and

water; and

wherein the propellant is present at about 5% to about 15% by weight of the formulation. In some embodiments, the ratio of all the ingredients other than propellant:propellant is from about 100:5 to about 100:15. In some embodiments, the ratio of carrier to propellant is about 100:5 to about 100:15.

Also disclosed herein, in various embodiments, are foams derived from the foamable formulations (i.e., compositions resulting from the foaming of the carrier by bringing it in contact with the propellant).

Exemplary foamable and foam formulations are further provided herein.

Foams typically exhibit low density, resulting from their bubbles structure, which includes fine or small air or gas bubbles. In contrast, ointments or gels do not include or hardly include entrapped air or gas bubbles and therefor exhibit higher density.

In some embodiments, a foam or foamable formulation as provided herein has a density or specific gravity of less than about 0.3 g/mL, depending on the composition and on the propellant concentration of the formulation. For the sake of comparison, an ointment or gel typically exhibits a density of about 1 g/mL. For example, the foam or foamable formulation may have a density of less than about 0.5 g/mL, for example, less than about 0.45 g/mL; less than about 0.4 g/mL, less than about 0.35 g/mL, less than about 0.3 g/mL, less than about 0.25 g/mL, less than about 0.2 g/mL; less than about 0.15 g/mL; less than about 0.1 g/mL, less than about 0.09 g/mL, less than about 0.08 g/mL, less than about 0.07 g/mL, less than about 0.06 g/mL, less than about 0.05 g/mL, less than about 0.04 g/mL, less than about 0.03 g/mL, less than about 0.02 g/mL, less than about 0.01 g/mL, or any density in between. In some embodiments, a foamable or foam formulation as provided herein has a density of between about 0.3 g/mL and about 0.01 g/mL. In some embodiments, a foam or foamable formulation as provided herein has a density of between about 0.05 g/mL and about 0.09 g/mL.

In some embodiments, density (mass per volume, i.e., g/mL) is measured by completely filling a pre-weighed tube having a known volume with a foam or foamable formulation and weighing the foam or foamable formulation in the filled tube. The mass is then calculated by subtracting the weight of the empty tube (i.e., the tube without the foam or foamable formulation) from the weight of the foam filled tube. Density measurement is provided for the calculated mass per the known volume of the tube.

### Surface Active Agents

For clarification, in the context herein the term “standard surfactant” or “customary surfactant” refers herein to customary non-ionic, anionic, cationic, zwitterionic, amphoteric, and amphiphilic surfactants. A fatty alcohol or a fatty acid and certain waxes (such as beeswax, hydrogenated castor oil and paraffin wax) are not regarded as a standard surfactant. However, in contrast, ethers or esters formed from such fatty alcohols or fatty acids can be regarded as a standard or customary surfactant. In some embodiments, the terms “surface active agent” and “surfactant” are used interchangeably.

Surfactants of all kinds are undesirable in accordance with the present invention, as they are generally known to possess irritation potential.

Thus, in some embodiments, a foam or foamable formulation as provided herein is surfactant free (i.e., comprises 0% by weight of the formulation of a surfactant). In some embodiments, a foam or foamable formulation as provided herein is substantially surfactant free. In some embodiments, a foam or foamable formulation as provided herein is substantially surfactant free when it comprises less than about 1%, less than about 0.5% by weight of the formulation of a surfactant, for example, less than about 0.4% by weight of the formulation; less than about 0.35% by weight of the formulation; less than about 0.3% by weight of the formulation; less than about 0.25% by weight of the formulation; less than about 0.2% by weight of the formulation; less than about 0.15% by weight of the formulation; less than about 0.1% by weight of the formulation; less than 0.05% by weight of the formulation; less than 0.025% by weight of the formulation, less than about 0.01%, or any percentage in between. In some embodiments, a foam or foamable formulation disclosed herein is free or substantially free of all surfactants. In some embodiments, a foam or foamable formulation disclosed herein is free or substantially free of all or certain classes of non-ionic, anionic, cationic, zwitterionic, amphoteric and/or amphiphilic surfactants.

Non-limiting examples of classes of non-ionic surfactants that are undesirable according to the present invention include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

As mentioned above, in the context of the present invention, while fatty alcohols, fatty acids, and certain waxes are somewhat amphiphilic, these substances may not be effective as stand-alone surfactants that can stabilize an emulsion, let alone foamable emulsion compositions, because of their very weak emulsifying capacity and further due to their weak foaming capacity on their own.

In certain embodiments, fatty alcohols, fatty acids, and certain waxes are occasionally used in a supporting role as co-emulsifiers, i.e., in combination with a standard surfactant. In this context, they are commonly used as thickeners and have successfully been used as foam adjuvants to assist customary surfactants to boost foam quality and stability. For the purposes of forming an emulsion they are usually regarded as an oil and thus have a “required” HLB value for the purpose of determining what standard surfactant might be appropriate to use with the oil phase.

Generally, surfactants are known to possess irritation potential. One way to try and reduce or minimize potential irritation and drying of the skin or mucosa due to surfactants and their repeated use, especially when formulations are to be left on the skin or mucosa rather than being washed off, is to use essentially or primarily nonionic surfactants at significant concentrations although preferably below 5%. The current disclosure uses hydroalcoholic formulations which produce quality breakable foam yet omit customary surfactants from a composition, which may contribute to improved tolerability of such a composition. This is especially so when a formulation is to be applied to a very sensitive target site, and particularly so on a repeated basis.

In certain embodiments, a foam or foamable composition described herein is free of customary surfactants or emulsifiers, or in other words “surfactant-free.” In certain embodiments, the foam or foamable composition is substantially free of customary surfactants or emulsifiers, or in other words “substantially surfactant-free,” i.e., less than 1% surfactant by weight, or less than 0.5%, or less than 0.1%, or less than 0.05%, or less than 0.01%, or any percentage in between.

In certain embodiments, a foam or foamable composition described herein is free or substantially free of a surfactant (e.g., less than 1%, 0.5%, 0.1%, 0.05%, 0.01% surfactant by weight, or any percentage in between). In certain embodiments, the composition is free or substantially free of an ionic surfactant. In certain embodiments, the composition is free or substantially free of a zwitterionic surfactant. In certain embodiments, the composition is free or substantially free of a non-ionic surfactant.

An emulsifier has three primary roles: (i) to reduce as much as possible the interfacial tension between the two liquid phases, (ii) to cover as efficiently as possible the dispersed droplets and prevent interaction with other droplets, and (iii) to desorb (opposite of absorb) from the interface as little as possible by anchoring deeply and efficiently into the dispersed phase and to dangle into the continuous phase so that the repulsive forces will be contained. In other words, once the emulsifier has been adsorbed at the interface it should remain there with high residence to stabilize the emulsion even when the repulsive forces of the continuous and dispersed phases are maximal.

Emulsions are best stabilized when the droplets dispersed in the continuous phase are fully covered by surfactant. Only surfactants that cover the interface and reside statistically most of their time around the droplets are considered efficient emulsifiers. Fatty alcohols do not meet this criterion as they have low residence around the droplets since they can reversibly move in and out of the interface and have a tendency to desorb more than they absorb.

In summary, the term surface active agent or surfactant is sometimes used loosely in the art and some publications may refer to compounds that have a supportive role, such as co-surfactants as surfactants. Substances which cannot function as true surfactants on their own but only in the context of being used with another surfactant are not considered to be surfactants for the purposes described herein. Thus, in the context herein, waxes listed above are not regarded as surfactants. Similarly, a fatty alcohol is not regarded as a surfactant, and likewise, a fatty acid is not regarded as a surfactant. In contrast, however, an ether or an ester formed from either a fatty acid or a fatty alcohol can be a surfactant. In addition, quaternary ammonium compounds and ions, which for example are often seen in hair preparations, are not regarded as surfactants.

### Other Components

In some embodiments, a foam or foamable formulation as provided herein does not comprise a carbomer and may be described as carbomer-free. In other embodiments, a foam or foamable formulation as provided herein may be substantially free of carbomer or essentially free of carbomer. As used herein, “substantially free of carbomer” means less than about 0.15%, or less than about 0.12%, or less than about 0.1%, or less than about 0.08%, or less than about 0.06%. In some embodiments, a formulation provided herein is essentially free of carbomer. As used herein, “essentially free of carbomer” means less than about 0.05%, or less than about 0.04%, or less than about 0.03%, or less than about 0.02%, or less than about 0.01%, or less than about 0.005% or having only trace amounts. In some embodiments, a foam or foamable formulation as provided herein is carbomer-free (i.e., comprises 0% by weight of the formulation of a carbomer).

In various embodiments, the active agent or pharmaceutically acceptable salt thereof is an antifungal agent. For example, in some embodiments, the antifungal agent is a polyene, an azole, an allylamine (for instance, naftifine or terbinafine), a benzyl amine (for instance, butenafine), or another antifungal agent (for instance, amorolfine), or a pharmaceutically acceptable salt thereof. Examples of polylenes include, e.g,. nystatin and Amphotericin B. Examples of azoles include miconazole, clotrimazole, ketoconazole, oxiconazole, eberconazole, econazole, sulconazole and sertaconazle bifonazole, butoconazole, fenticonazole, isoconazole, omoconazole, and tioconazole, or pharmaceutically acceptable salts thereof. Exemples of other antifungal agents include naftifine, terbinafine, and butenafine, or pharmaceutically acceptable salts. Still other antifungal agents include, e.g., ciclopirox or selenium sulfide. Additional antifungals include agents that block NA synthesis including, e.g., flucytosine, and those that disrupt microtubule function including, e.g., griseofulvin. Suitable antifungals can include one or more of candicidin, filipin, hamycin, natamycin, and rimocidin. Triazoles, including albaconazole, fluconazole, isavuconazole, itraconazole, posaconazole, ravuconazole, terconazole, and voriconazole are also suitable antifungal active agents. Also suitable are thiazoles including, e.g., abafungin. Suitable antifungal agents include, e.g., amorolfin, butenafine, naftifine, and terbinafine. In addition, echinocandins, including anidulafungin, caspofungin, and micafungin, are suitable antifungals. Also suitable are griseofulvin, benzoic acid, ciclopirox, haloprogin, polygodial, tolnaftate, undecylenic acid, and Crystal violet.

In some embodiments, the antifungal agent is an allylamine. In some embodiments, the antifungal agent is naftifine. In some embodiments, the antifungal agent is terbinafine.

In some embodiments, the active agent is present as a pharmaceutically acceptable salt. As used herein, the phrase “pharmaceutically acceptable salt” refers to salts of certain ingredient(s) which possess the same activity as the unmodified compound(s) and which are neither biologically nor otherwise undesirable. A salt can be formed with, for example, organic or inorganic acids. Exemplary suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

In some embodiments, the pharmaceutically acceptable salt is an acid salt. In some embodiments the pharmaceutically acceptable salt is a hydrochloride salt.

Naftifine ((E)-N-methyl-N-(1-naphthyl-methyl)-3-phenyl-2-propen-l-amine hydrochloride) is an allylamine derivative and has been shown to have antifungal properties. Naftifine, however, is a weak base and is poorly soluble when it is found in the nonionized form.

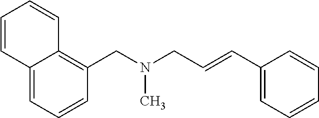 [SMILES: CN(C/C=C/c1ccccc1)Cc1cccc2ccccc12]

In various embodiments, naftifine or a pharmaceutically acceptable salt thereof is present in the disclosed foam or foamable formulation at about 2% to about 4% by weight of the carrier. For example, about 2.5% to about 3.5% by weight of the carrier; about 3% to about 4% by weight of the carrier; about 2% to about 3% by weight of the carrier; about 2.5% to about 3% by weight of the carrier; about 3% to about 3.5% by weight of the carrier; or any percentage in between. In some embodiments, naftifine or a pharmaceutically acceptable salt thereof is present at about 4% by weight of the carrier. In some embodiments, naftifine or a pharmaceutically acceptable salt thereof is present at about 3.5% by weight of the carrier. In some embodiments, naftifine or a pharmaceutically acceptable salt thereof is present at about 3% by weight of the carrier. In some embodiments, naftifine or a pharmaceutically acceptable salt thereof is present at about 2.5% by weight of the carrier. In some embodiments, naftifine or a pharmaceutically acceptable salt thereof is present at about 2% by weight of the carrier.

In some embodiments, the pharmaceutically acceptable salt form of naftitine is naftifine hydrochloride. In various embodiments, Naftifine hydrochloride is present in the disclosed foam or foamable formulation at about 2% to about 4% by weight of the carrier. For example, about 2.5% to about 3.5% by weight of the carrier; about 3% to about 4% by weight of the carrier; about 2% to about 3% by weight of the carrier; about 2.5% to about 3% by weight of the carrier; about 3% to about 3.5% by weight of the carrier; or any percentage in between. In some embodiments, naftifine hydrochloride is present at about 4% by weight of the carrier. In some embodiments, naftifine hydrochloride is present at about 3.5% by weight of the carrier. In some embodiments, naftifine hydrochloride is present at about 3% by weight of the carrier. In some embodiments, naftifine hydrochloride is present at about 2.5% by weight of the carrier. In some embodiments, naftifine hydrochloride is present at about 2% by weight of the carrier.

The foam or foamable compositions as provided herein may, in some embodiments, contain C₁₋₆ alkyl alcohols.

Exemplary C₁₋₆ alkyl alcohols include, but are not limited to, one or more of methanol, ethanol, n-propyl alcohol, isopropyl alcohol, n-butyl alcohol, t-butyl alcohol, 2-butanol, iso-butanol, pentanol, hexanol, and cyclohexanol. In some embodiments, the C₁₋₆ alkyl alcohol is methanol, ethanol, n-propyl alcohol, or isopropyl alcohol. In some embodiments, the C₁₋₆ alkyl alcohol is ethanol. In some embodiments, an C₁₋₆ alkyl alcohol is a mixture of two or more such alcohols, e.g., a mixture comprising ethanol and isopropyl alcohol, or ethanol and methanol, or ethanol and n-propyl alcohol.

As used herein and unless otherwise indicated, the term “ethanol” refers to USP grade ethanol. In some embodiments, the USP grade ethanol is USP grade dehydrated ethanol (i.e., ethanol absolute). In some embodiments, USP grade ethanol is ethanol containing NLT 94.9% and NMT 96.0%, by volume. In some embodiments, USP grade dehydrated ethanol is ethanol containing NLT 99.2% and NMT 99.5%, by volume.

In some embodiments, the C₁₋₆ alkyl alcohol is present in the foam or foamable formulation at about 15% to about 35% by weight of the carrier. For example, about 15% to about 30% by weight of the carrier; about 15% to about 25% by weight of the carrier; about 20% to about 35% by weight of the carrier; about 25% to about 35% by weight of the carrier, or any percentage in between. In some embodiments, the C₁₋₆ alkyl alcohol is present in the foam or foamable formulation at about 18% to about 22% by weight of the carrier. In some embodiments, the C₁₋₆ alkyl alcohol is present in the foam or foamable formulation at about 20% by weight of the carrier. In some embodiments, the C₁₋₆ alkyl alcohol is present in the foam or foamable formulation at about 28% to about 32% by weight of the carrier. In some embodiments, the C₁₋₆ alkyl alcohol is present in the foam or foamable formulation at about 30% by weight of the carrier. In some embodiments, the C₁₋₆ alkyl alcohol is present in the foam or foamable formulation at about 30% by weight of the carrier. In some embodiments, the C₁₋₆ alkyl alcohol is present in the foam or foamable formulation at about 25% by weight of the carrier. In some embodiments, the C₁₋₆ alkyl alcohol is present in the foam or foamable formulation at about 20% by weight of the carrier. In some embodiments, the C₁₋₆ alkyl alcohol is present in the foam or foamable formulation at about 15% by weight of the carrier. The foam or foamable formulations provided herein may be described as hydroalcoholic in view of the presence of the amounts of a C₁₋₆ alkyl alcohol in the formulation.

In some embodiments, ethanol is present in the foam or foamable formulation at about 15% to about 35% by weight of the carrier. For example, about 15% to about 30% by weight of the carrier; about 15% to about 25% by weight of the carrier; about 20% to about 35% by weight of the carrier; about 25% to about 35% by weight of the carrier, or any percentage in between. In some embodiments, ethanol is present in the foam or foamable formulation at about 18% to about 22% by weight of the carrier. In some embodiments, ethanol is present in the foam or foamable formulation at about 20% by weight of the carrier. In some embodiments, ethanol is present in the foam or foamable formulation at about 28% to about 32% by weight of the carrier. In some embodiments, ethanol is present in the foam or foamable formulation at about 30% by weight of the carrier.

The foam or foamable compositions provided herein may, in some embodiments, further contain an alkylene glycol.

Exemplary alkylene glycols include, but are not limited to, one or more of ethylene glycol, propylene glycol, hexylene glycol, and butylene glycol. In some embodiments, the alkylene glycol is propylene glycol.

In some embodiments, the alkylene glycol is present in the foam or foamable formulation at about 15% to about 30% by weight of the carrier. For example, about 15% to about 25% by weight of the carrier; about 15% to about 20% by weight of the carrier; about 20% to about 30% by weight of the carrier; about 20% to about 25% by weight of the carrier; about 25% to about 30% by weight of the carrier, or any percentage in between. In some embodiments, the alkylene glycol is present in the foam or foamable formulation at about 15% to about 20% by weight of the carrier. In some embodiments, the alkylene glycol is present in the foam or foamable formulation at about 19% by weight of the carrier. In some embodiments, the alkylene glycol is present in the foam or foamable formulation at about 25% to about 30% by weight of the carrier. In some embodiments, the alkylene glycol is present in the foam or foamable formulation at about 28.5% by weight of the carrier.

In some embodiments, propylene glycol is the alkylene glycol. In some embodiments, the alkylene glycol comprises a mixture of glycols. In some embodiments, the glycols are mixed at a ratio of from about 3:1 to about 1:3 by weight. In some embodiments, the mixture of glycols comprises propylene glycol and ethylene glycol. In some embodiments, the propylene glycol and ethylene glycol are mixed at a ratio of about 2:1 by weight.

In some embodiments, propylene glycol is present in the foam or foamable formulation at about 15% to about 30% by weight of the carrier. For example, about 15% to about 25% by weight of the carrier; about 15% to about 20% by weight of the carrier; about 20% to about 30% by weight of the carrier; about 20% to about 25% by weight of the carrier; about 25% to about 30% by weight of the carrier, or any percentage in between. In some embodiments, propylene glycol is present in the foam or foamable formulation at about 15% to about 20% by weight of the carrier. In some embodiments, propylene glycol is present in the foam or foamable formulation at about 19% by weight of the carrier. In some embodiments, the propylene glycol is present in the foam or foamable formulation at about 25% to about 30% by weight of the carrier. In some embodiments, propylene glycol is present in the foam or foamable formulation at about 28.5% by weight of the carrier.

In some embodiments, the foam or foamable formulation comprises one or more of ethanol, dimethyl sulfoxide, and polyethylene glycol 200 in addition to or in place of propylene glycol. In some embodiments, when the formulation comprises one or more of ethanol, dimethyl sulfoxide, and polyethylene glycol 200 in place of propylene glycol, the amount of the one or more of ethanol, dimethyl sulfoxide, and polyethylene glycol 200 may be similar to, or about the same as the herein disclosed amounts of propylene glycol.

In some embodiments, a foam or foamable formulation described herein may comprise a thickening agent. In some embodiments, the thickening agent can be a polymer or a polymeric agent. As used herein the term “polymer” is interchangeable with the term “polymeric agent”. In some embodiments, the polymer or polymeric agent is present in the foam or foamable formulation at about 0.1% to about 5% by weight of the carrier. In some embodiment, the polymer or polymeric agent is present in the foam or foamable formulation at about 1% to about 4% by weight of the carrier. In some embodiment, the polymer or polymeric agent is present in the foam or foamable formulation at about 1% to about 3% by weight of the carrier. In some embodiment, the polymer or polymeric agent is present in the foam or foamable formulation at about 1% to about 2% by weight of the carrier. In some embodiment, the polymer or polymeric agent is present in the foam or foamable formulation at about 1.5% by weight of the carrier. In some embodiment, the polymer or polymeric agent is present in the foam or foamable formulation at about 1.75% by weight of the carrier.

Exemplary polymers or polymeric agents include, but are not limited to one or more naturally-occurring polymeric materials, such as a locust bean gum, sodium alginate, sodium caseinate, an egg albumin, a gelatin agar, a carrageenin gum, sodium alginate, an xanthan gum, a quince seed extract, a tragacanth gum, a guar gum, cationic guars, a hydroxypropyl guar gum, a starch, amine-bearing polymers such as chitosan; acidic polymers obtainable from natural sources, such as alginic acid and a hyaluronic acid; chemically modified starches and the like; carboxyvinyl polymers, a polyvinylpyrrolidone, a polyvinyl alcohol, polyacrylic acid polymers, polymethacrylic acid polymers, polyvinyl acetate polymers, polyvinyl chloride polymers, a polyvinylidene chloride polymers and the like, or cellulose ethers, such as a methylcellulose, a hydroxypropyl cellulose, a hydroxypropyl methylcellulose, a hydroxyethyl cellulose, a hydroxyl propylmethyl cellulose, a methylhydroxyethylcellulose, a methylhydroxypropylcellulose, a hydroxyethylcarboxymethylcellulose, a carboxymethyl cellulose, a carboxymethylcellulose, and a carboxymethylhydroxyethylcellulose.

In some embodiments, the polymer is a water gelling agent. As used herein, a “water gelling agent” is an agent that, when added to water in a sufficient concentration (e.g., up to about 5%), a gel is formed. In some embodiments, the water gelling agent is strong, such that about 1% or less of the gelling agent is sufficient to form a gel.

In some embodiments, the polymer is a cellulose ether. In some embodiments, the polymer is a hydroxypropyl cellulose.

One of skill in the relevant chemical and pharmaceutical arts will appreciate that cellulose ethers, and particularly hydroxycelluloses are available in a variety of chain lengths and that the amount required by a particular formulation may vary depending on, inter alia, the chain length of the selected cellulose ether. In some embodiments, the hydroxypropyl cellulose has a viscosity of between about 100 cP and about 25000 cP. In some embodiments, the hydroxypropyl cellulose has a viscosity of between about 1500 cP and about 2500 cP. In some embodiments, the hydroxypropyl cellulose has a viscosity of between about 1500 cP and about 3000 cP. In some embodiments, the hydroxypropyl cellulose has a viscosity of between about 4000 cP and about 6500 cP. In some embodiments, the hydroxypropyl cellulose has a viscosity of between about 200 cP and about600 cP. In some embodiments, the hydroxypropyl cellulose has a viscosity of between about 150 cP and about 400 cP. In some embodiments, the hydroxypropyl cellulose has a viscosity of between about 75 cP and about150 cP.

The concentration of the polymer or polymeric agent should be selected so that the foamable formulation, after filling into aerosol canisters and pressurized with propellant, is flowable so the foamable formulation can be released from the canister. In further embodiments, the foamable formulation should be such that it can be shaken in the canister. In some embodiments, the concentration of the polymeric agent is selected such that the viscosity of the foamable formulation, prior to the filling of the foamable formulation into aerosol canisters or prior to the addition of propellant, is less than about 30,000 cP, or is less than about 15,000 cP, or is less than about 13,000 cP, or is less than about 11,000 cP, or is less than about 9,000 cP , or is less than about 7,000 cP, or is less than about 5,000 cP, or is less than about 4,000 cP, or is less than about 3,000 cP, or is less than about 2,000 cP, or any centipoise unit in between. In some embodiments, the viscosity is between about 10 cP and about 30,000 cP. In some embodiments, the viscosity is between about 20 cP and about 30,000 cP. In some embodiments, the viscosity is between about 30 cP and about 30,000 cP. In some embodiments, the viscosity is between about 40 cP and about 30,000 cP. In some embodiments, the viscosity is between about 50 cP and about 30,000 cP. In some embodiments, the viscosity is between about 60 cP and about 30,000 cP. In some embodiments, the viscosity is between about 70 cP and about 30,000 cP. In some embodiments, the viscosity is between about 80 cP t and o about 30,000 cP. In some embodiments, the viscosity is between about 90 cP and about 30,000 cP. In some embodiments, the viscosity is between about 100 cP and about 30,000 cP. In some embodiments, the viscosity is between about 120 cP and about 20,000 cP. In some embodiments, the viscosity is between about 130 cP and about 15,000 cP. In some embodiments, the viscosity is between about 140 cP and about 10,000 cP. In some embodiments, the viscosity is between about 150 cP and about 7,000 cP. In some embodiments, the viscosity is between about 160 cP and about 6,000 cP. In some embodiments, the viscosity is between about 170 cP and about 5,000 cP. In some embodiments, the viscosity is between about 180 cP and about 4,000 cP. In some embodiments, the viscosity is between about 190 cP and about 3,000 cP. In some embodiment,s the viscosity is between about 200 cP and about 2,000 cP. In some embodiments, the viscosity is between about 210 cP and about 1500 cP. In some embodiments, the viscosity is between about 220 cP and about 1000 cP. In some embodiments, the viscosity is between about 230 cP and about 900 cP. In some embodiments, the viscosity is between about 200 cP and about 800 cP. In some embodiments, the viscosity is between about 240 cP and about 800 cP. In some embodiments, the viscosity is between about 250 cP and about 700 cP. In some embodiments, the viscosity is between about 260 cP and about 650 cP. In some embodiments, the viscosity is between about 400 cP and about 600 cP. In some embodiments, the viscosity is between about 430 cP and about 570 cP. In some embodiments, the viscosity is between about 270 cP and about 550 cP. In some embodiments, the spreadability of a foam or foamable formulation improves with a lower viscosity and/or a lower density of the formulation.

In some embodiments, the polymer is present in the foam or foamable formulation at about 1% to about 2% by weight of the carrier. For example, about 1.5% to about 2% by weight of the carrier; about 1% to about 1.5% by weight of the carrier; about 1.25% to about 1.75% by weight of the carrier, or any percentage in between. In some embodiments, the polymer is present in the foam or foamable formulation at about 1.5% to about 2% by weight of the carrier. In some embodiments, the polymer is present in the foam or foamable formulation at about 1.75% by weight of the carrier.

In some embodiments, the polymer is selected from the group consisting of a methylcellulose, a hydroxypropyl cellulose, a hydroxypropyl methylcellulose, a hydroxyethyl cellulose, a hydroxyl propylmethyl cellulose, a methylhydroxyethylcellulose, a methylhydroxypropylcellulose, a hydroxyethylcarboxymethylcellulose, a carboxymethyl cellulose, a carboxymethylcellulose, a carboxymethylhydroxyethylcellulose, and mixtures of any two or more thereof. In some embodiments, the polymer comprises a hydroxylpropyl cellulose. In some embodiments, the hydroxypropyl cellulose is present in the foam or foamable formulation at about 1% to about 2% by weight of the carrier. For example, about 1.5% to about 2% by weight of the carrier; about 1% to about 1.5% by weight of the carrier; about 1.25% to about 1.75% by weight of the carrier; or any percentage in between. In some embodiments, hydroxypropyl cellulose is present in the foam or foamable formulation at about 1.5% to about 2% by weight of the carrier. In some embodiments, hydroxypropyl cellulose is present in the formulation at about 1.75% by weight of the carrier.

The foam or foamable compositions provided herein may, in some embodiments, further comprise a long chain fatty alcohol. A long chain fatty alcohol as used herein has 14 to 22 carbon atoms in its carbon chain. In some embodiments, the long chain fatty alcohol has 16 to 22 carbon atoms in its carbon chain. In some embodiments, the long chain fatty alcohol is selected from the group consisting of fatty alcohols having 15 or more carbons in their carbon chain, such as cetyl alcohol and stearyl alcohol (or mixtures thereof, i.e. cetostearyl alcohol). Other examples of long chain fatty alcohols include, but are not limited to, myristyl alcohol (C14), arachidyl alcohol (C20), and behenyl alcohol (C22). In some embodiments, the long chain fatty alcohol is cetyl alcohol, stearyl alcohol, behenyl alcohol, myristyl alcohol, or mixtures of two or more thereof. In some embodiments, the long chain fatty alcohol is cetostearyl alcohol. In some embodiments, the long chain fatty alcohol chain is a saturated long chain fatty alcohol. In some embodiments, the long chain fatty alcohol is an unsaturated long chain fatty alcohol. In some embodiments, the long chain fatty alcohol is branched. In some embodiments, the long chain fatty alcohol is a mixture of two or more fatty alcohols. In some embodiments, the foam or foamable formulation comprises a medium chain fatty alcohol having a chain length below C14, for example, in some embodiments, the medium chain fatty alcohol can have C14 to C8 carbon atoms in its carbon chain. In some embodiments, the foam or foamable formulation comprises very long chain fatty alcohols having a carbon chain of more than C22 carbon atoms, for example, in some embodiments, the very long chain fatty alcohol can have C24 to C50 carbon atoms in its carbon chain.

In some embodiments, the long chain fatty alcohol is present in the foam or foamable formulation at about 0.5% to about 2.0% by weight of the carrier. For example, about 0.5% to about 1.5% by weight of the carrier; about 0.75% to about 1.25% by weight of the carrier; or about 1.0% to about 2% by weight of the carrier, or any percentage in between. In some embodiments, the long chain fatty alcohol is present in the foam or foamable formulation at about 1.0% to about 2.0% by weight of the carrier. In some embodiments, the long chain fatty alcohol is present at about 1.0% by weight of the carrier. In some embodiments, the long chain fatty alcohol is present at about 1.5% by weight of the carrier.

In some embodiments, cetostearyl alcohol is present in the foam or foamable formulation at about 0.5% to about 2.0% by weight of the carrier. For example, about 0.5% to about 1.5% by weight of the carrier; about 0.75% to about 1.25% by weight of the carrier; or about 1.0% to about 2% by weight of the carrier, or any percentage in between. In some embodiments, cetostearyl alcohol is present in the foam or foamable formulation at about 1.0% to about 2.0% by weight of the carrier. In some embodiments, cetostearyl alcohol is present at about 1.0% by weight of the carrier. In some embodiments, cetostearyl alcohol is present at about 1.5% by weight of the carrier.

pH adjusters for use in the provided foam or foamable formulation can include any pharmaceutically acceptable composition, compound, or agent, suitable for adjusting the pH of the presently described topical pharmaceutical formulations without negatively affecting any property thereof. Suitable pH adjusters can include any pharmaceutically acceptable acid or base. In some embodiments, the pH adjustor comprises a basic pH adjuster. For example, in some embodiments, the pH adjuster is an amine base. Exemplary amine bases are known in the chemical and pharmaceutical arts and include, for example, triethanolamine (i.e., Trolamine). In some embodiments, a basic pH adjuster is a hydroxide. In some embodiments, the hydroxide is in the form of a salt of an alkali or alkaline earth metal. For example, a hydroxide salt can be selected from sodium hydroxide, potassium hydroxide, and calcium hydroxide. In some embodiments, a pH adjuster is a carbonate. In some embodiments, the carbonate is in the form of a salt of an alkali or alkaline earth metal. For example, a carbonate salt can be selected from sodium carbonate, potassium carbonate, and calcium carbonate.

In some embodiments, the described pH adjusters can be present in the foam or foamable formulations in an amount of about 0.01% to about 1% by weight of the carrier. For example, about 0.05% to about 1% by weight of the carrier; about 0.05% to about 0.5% by weight of the carrier; about 0.08% to about 0.4% by weight of the carrier; about 0.08% to about 0.35% by weight of the carrier; about 0.08% to about 0.3% by weight of the carrier; about 0.08% to about 0.25% by weight of the carrier; about 0.09% to about 0.4% by weight of the carrier; about 0.09% to about 0.3% by weight of the carrier; about 0.09% to about 0.25% by weight of the carrier; about 0.1% to about 0.25% by weight of the carrier; about 0.11% to about 0.24% by weight of the carrier; about 0.12% to about 0.23% by weight of the carrier; about 0.13% to about 0.22% by weight of the carrier; about 0.13% to about 0.21% by weight of the carrier; about 0.13% to about 0.20% by weight of the carrier; about 0.13% to about 0.19% by weight of the carrier; about 0.13% to about 0.18% by weight of the carrier; about 0.13% to about 0.17% by weight of the carrier; about 0.14% to about 0.21% by weight of the carrier; about 0.15% to about 0.2% by weight of the carrier; about 0.16% to about 0.19% by weight of the carrier; about 0.16% to about 0.18% by weight of the carrier; about 0.165% to about 0.175% by weight of the carrier; or any percentage in between. In some embodiments, the pH adjustor is present in the foam or foamable formulation in an amount of about 0.14% by weight of the carrier. In some embodiments, the pH adjustor is present in the foam or foamable formulation in an amount of about 0.15% by weight of the carrier. In some embodiments, the pH adjustor is present in the foam or foamable formulation in an amount of about 0.16% by weight of the carrier. In some embodiments, the pH adjustor is present in the foam or foamable formulation in an amount of about 0.17% by weight of the carrier. In some embodiments, the pH adjustor is present in the foam or foamable formulation in an amount of about 0.18% by weight of the carrier.

In some embodiments, the pH adjuster comprises trolamine. Trolamine can be present in the foam or foamable formulations in an amount of about 0.01% to about 1% by weight of the carrier. For example, about 0.05% to about 1% by weight of the carrier; about 0.05% to about 0.5% by weight of the carrier; about 0.08% to about 0.4% by weight of the carrier; about 0.08% to about 0.35% by weight of the carrier; about 0.08% to about 0.3% by weight of the carrier; about 0.08% to about 0.25% by weight of the carrier; about 0.09% to about 0.4% by weight of the carrier; about 0.09% to about 0.3% by weight of the carrier; about 0.09% to about 0.25% by weight of the carrier; about 0.1% to about 0.25% by weight of the carrier; about 0.11% to about 0.24% by weight of the carrier; about 0.12% to about 0.23% by weight of the carrier; about 0.13% to about 0.22% by weight of the carrier; about 0.13% to about 0.21% by weight of the carrier; about 0.13% to about 0.20% by weight of the carrier; about 0.13% to about 0.19% by weight of the carrier; about 0.13% to about 0.18% by weight of the carrier; about 0.13% to about 0.17% by weight of the carrier; about 0.14% to about 0.21% by weight of the carrier; about 0.15% to about 0.2% by weight of the carrier; about 0.16% to about 0.19% by weight of the carrier; about 0.16% to about 0.18% by weight of the carrier; about 0.165% to about 0.175% by weight of the carrier; or any percentage in between. In some embodiments, trolamine is present in the foam or foamable formulation in an amount of about 0.14% by weight of the carrier. In some embodiments, trolamine is present in the foam or foamable formulation in an amount of about 0.15% by weight of the carrier. In some embodiments, trolamine is present in the foam or foamable formulation in an amount of about 0.16% by weight of the carrier. In some embodiments, trolamine is present in the foam or foamable formulation in an amount of about 0.17% by weight of the carrier. In some embodiments, trolamine is present in the foam or foamable formulation in an amount of about 0.18% by weight of the formulation.

In some embodiments, a foamable formulation provided herein has a pH of from about 4.0 to about 7.5; from about 4.0 to about 7.0; from about 4.0 to about 6.5; from about 4.0 to about 6.0; from about 4.5 to about 6.5; from about 4.5 to about 6.0; from about 4.5 to about 5.5; from about 4.7 to about 5.5;; from about 4.8 to about 5.4; from about 4.7 to about 5.5, from about 4.9 to about 5.3; from about 4.5 to about 6.0; from about 4.6 to about 5.9; from about 4.7 to about 5.8; from about 4.8 to about 5.7; from about 4.9 to about 5.6; from about 5.0 to about 5.4; from about 5.1 to about 5.3; from about 5.5 to about 7.5; from about 5.6 to about 7.4; from about 5.7 to about 7.3; from about 5.8 to about 7.2; from about 5.9 to about 7.1; from about 6.0 to about 7.0; from about 6.1 to about 6.9; from about 6.2 to about 6.8; from about 6.3 to about 6.7; from about 6.4 to about 6.6; about 4.5; about 4.6; about 4.7; about 4.8; about 4.9; about 5.0; about 5.1; about 5.2; about 5.3; about 5.4; about 5.5; about 5.6; about 5.7; about 5.8; about 5.9; about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. For example, the pH of a foamable formulation provided herein comprising 3% by weight of the foam or foamable formulation of naftifine hydrochloride at 23° C. ±2° C. can be from about 4.0 to about 6.0 or from about 4.5 to about 6.0. In some embodiments, a foam or foamable formulation provided herein has a pH of from about 4.0 to about 6.0. In some embodiments, a foam or foamable formulation provided herein has a pH of from about 4.5 to about 6.5.

Chelating agents for use in the provided foam or foamable formulation can include any known pharmaceutically acceptable chelating agents. Suitable chelating agents include, but are not limited to, any one or more of ethylenediaminetetraacetic acid (EDTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), polyphosphates, porphine, and any pharmaceutically acceptable salt thereof. In some embodiments, a suitable chelating agent is disodium EDTA (i.e., edetate disodium).

The chelating agents can be present in the provided foam or foamable formulations in an amount of, for example, about 0.001% to about 10% by weight of the carrier; about 0.005% to about 5% by weight of the carrier; about 0.005% to about 1% by weight of the carrier; about 0.005% to about 0.5% by weight of the carrier; about 0.001% to about 1% by weight of the carrier; about 0.01% to about 5% by weight of the carrier; about 0.01% to about 0.3% by weight of the carrier; about 0.006% to about 0.04% by weight of the carrier; about 0.007% to about 0.035% by weight of the carrier; about 0.008% to about 0.035% by weight of the carrier; about 0.009% to about 0.035% by weight of the carrier; about 0.01% to about 0.03% by weight of the carrier; about 0.015% to about 0.025% by weight of the carrier; about 0.018% to about 0.022% by weight of the carrier; or about 0.019% to about 0.021% by weight of the carrier, or any percentage in between. In some embodiments, the chelating agent is present at about 0.01% to about 0.3% by weight of the carrier. In some embodiments, the chelating agent is present at about 0.019% by weight of the carrier. In some embodiments, the chelating agent is present at about 0.020% by weight of the carrier. In some embodiments, the chelating agent is present at about 0.021% by weight of the carrier.

Disodium EDTA (i.e., edetate disodium) can be present in the provided foam or foamable formulations in an amount of, for example, about 0.001% to about 10% by weight of the carrier; about 0.005% to about 5% by weight of the carrier; about 0.005% to about 0.5% by weight of the carrier; about 0.001% to about 1% by weight of the carrier; about 0.01% to about 5% by weight of the carrier; about 0.01% to about 0.3% by weight of the carrier; about 0.006 to about 0.04% by weight of the carrier; about 0.007 to about 0.035% by weight of the carrier; about 0.008% to about 0.035% by weight of the carrier;

about 0.009% to about 0.035% by weight of the carrier; about 0.01% to about 0.03% by weight of the carrier; about 0.015% to about 0.025% by weight of the carrier; about 0.018% to about 0.022% by weight of the carrier; about 0.019% to about 0.021% by weight of the carrier, or any percentage in between. In some embodiments, disodium EDTA (i.e., edetate disodium) is present at about 0.01% to about 0.3% by weight of the carrier. In some embodiments, disodium EDTA (i.e., edetate disodium) is present at about 0.019% by weight of the carrier. In some embodiments, disodium EDTA (i.e., edetate disodium) is present at about 0.020% by weight of the carrier. In some embodiments, disodium EDTA (i.e., edetate disodium) is present at about 0.021% by weight of the carrier.

Water may be included in the provided foam or foamable formulations and can be present in amounts to bring the total carrier composition to 100% by weight. For example, water can be present in the provided foam or foamable formulations at an amount of about 10% to about 75% by weight of the carrier; about 25% to about 75% by weight of the carrier, about 40% to about 70% by weight of the carrier, or about 50% to about 60% by weight of the carrier, or any percentage in between. In some embodiments, water is present at about 52% to about 56% by weight of the carrier (e.g., about 54% by weight of the carrier or about 55% by weight of the carrier). In some embodiments, water is present at about 32% to about 38% by weight of the carrier (e.g., about 34% by weight of the carrier or about 35% by weight of the carrier). As used herein, foams and foamable formulations comprising these amounts of water can be described as aqueous formulations because water is a primary component of the formulation.

In some embodiments, a foam or foamable formulation described herein comprises a propellant. Suitable propellants include volatile hydrocarbons such as butane, propane, isobutane or mixtures thereof. In some embodiments, a hydrocarbon mixture AP-70 is used. In some embodiments, a lower pressure hydrocarbon mixture AP-46 is used. Both AP-70 and AP-46 contain butane, propane, and isobutane although in different proportions. AP-46 is composed of about 16% w/w of propane, about 82% w/w of isobutane and about 2% w/w of propane. AP-70 is composed of about 50% w/w of propane, about 20% w/w of isobutene and about 30% w/w of propane. Hydrofluorocarbon (HFC) propellants are also suitable as propellants in the context disclosed herein. Exemplary HFC propellants include 1,1,1,2-tetrafluorethane (Dymel 134), and 1,1,1,2,3,3,3 heptafluoropropane (Dymel 227). Dimethyl ether is also a suitable propellant. In some embodiments, compressed gas (e.g., air, carbon dioxide, nitrous oxide, and nitrogen) may be used as a suitable propellant.

In some embodiments, a combination of at least two propellants, selected from HFC, hydrocarbon propellants, dimethyl ether and compressed gases may be used in the foamable formulations provided herein.

Any concentration of the propellant which affords administrable foam is useful in accordance with the present invention. In some embodiments, in preparing the foam or foamable formulations, all the ingredients other than propellant are combined to 100% and the propellant is added thereafter. For example, if all the ingredients other than propellant amount to 100 grams by weight, and 10 grams of propellant are subsequently added, then the ratio of all ingredients other than propellant:propellant is 100:10. Likewise, if the carrier amounts to 100 grams and 10 grams of propellant is added then the ratio of carrier to propellant is 100:10. In some embodiments, all % values are provided on a weight (w/w) basis, based on the foam or foamable formulation without propellant. In some embodiments, the propellant is present in the foam or foamable formulations at a concentration from about 3% to about 25% by weight of the overall formulation. For example, about 3% to about 18% by weight of the formulation; about 4% to about 22% by weight of the foamable formulation; or about 3% to about 12% by weight of the formulation, or any percentage in between.

In some embodiments, the propellant is present in the foam or foamable formulations at a concentration from about 5% to about 15% by weight of the formulation. For example, about 5% to about 10% by weight of the formulation; about 10% to about 15% by weight of the formulation; or about 6% to about 12% by weight of the formulation. In some embodiments, the propellant is present at about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25% by weight of the foam or foamable formulation. In some embodiments, the propellant is present at about 8% by weight of the foam or foamable formulation.

In some embodiments, the propellant can also be used to expel the foamable formulation using a bag in can system or a can in can system as will be appreciated by someone skilled in the art. In some embodiments, part of the propellant system is in the foamable formulation and part of the propellant system is separated from the foamable formulation. In this way it is possible to reduce the amount of propellant in the foamable formulation but still provide good expulsion from the canister, where the foamable formulation is expelled quickly but without jetting or noise.

In some embodiments, a foam or foamable formulation provided herein further comprises a preservative. The preservatives for use in the presently described foam or foamable formulations can include any known pharmaceutically acceptable preservative that functions by inhibiting bacteria, fungi, yeast, mold, other microbe, and/or by inhibiting oxidation. Suitable preservatives can include but are not limited to one or more of antimicrobial agents and/or antioxidants. Suitable antimicrobial agents can include, but are not limited to, one or more of benzoates, benzyl alcohol, sodium benzoate, sorbates, propionates, and nitrites. Suitable antioxidants can include, but are not limited to, one or more of vitamin C, butylated hydroxytoluene (BHT), sulphites, and vitamin E In some embodiments, the preservative is benzyl alcohol.

In some embodiments, the preservative is present in the foam or foamable formulation at about 0.5% to about 1.5% by weight of the carrier. For example, the preservative is present at about 0.75% to about 1.25% by weight of the carrier. In some embodiments, the preservative is present at about 1% by weight of the carrier.

In some embodiments, benzyl alcohol is present in the foam or foamable formulation at about 0.5% to about 1.5% by weight of the carrier. For example, benzyl alcohol is present at about 0.75% to about 1.25% by weight of the carrier. In some embodiments, benzyl alcohol is present at about 1% by weight of the carrier.

In some embodiments, a foam or foamable formulation provided herein is substantially preservative free. As used here, the term “substantially preservative free” refers to a foam or foamable formulation comprising less than about 0.5%, or less than about 0.4%, or less than about 0.3%, or less than about 0.2%, or less than about 0.1% of a preservative.

In some embodiments, a foam or foamable formulation provided herein is essentially preservative free. In some embodiments, a foam or foamable formulation provided herein is essentially free of one or more, or all of, the preservatives disclosed herein. As used herein, the term “essentially preservative free” refers to a foam or foamable formulation comprising less than about 0.05%, or less than about 0.04%, or less than about 0.03%, or less than about 0.02%, or less than about 0.01%, or less than about 0.005%, or having only trace amounts of a preservative. In some embodiments, a foam or foamable formulation provided herein is preservative free.

In some embodiments, a foam or foamable formulation disclosed herein includes one or more additional components. Such additional components include but are not limited to one or more of bulking agents, diluents, humectants, moisturisers, keratolytic agents, pH preservatives, pH adjusters, protectants, skin penetration or permeation enhancers, solubilizers, penetration enhancers, sunscreens, sun blocking agents, and viscosity modifiers. As is known to one skilled in the art, in some instances a specific additional component may have more than one activity, function or effect.

In some embodiments, a foam or foamable formulation as provided herein is applied to a subject and is very easy to use. When applied onto the afflicted body surface of mammals, i.e., humans or animals, it is in a foam state, allowing free application without spillage. Upon further application of a mechanical force, e.g., by rubbing the composition onto the body surface, the formulation spreads on the surface and is absorbed. In some embodiments, the rubbing action can be gentle and a simple rub may suffice. In some embodiments, the foam will collapse on the application of a mechanical or shear force. In some embodiments, a low, mild, or gentle force may be sufficient to facilitate the foam's collapse. In some embodiments, most or all of the foam collapses upon application of a mechanical or shear force. In some embodiments, the foam spreads and collapses upon application of a shear or mechanical force.

In some embodiments, the absorption of the foam or foamable formulation described herein is rapid. In some embodiments, the majority of the formulation (e.g., more than about 50% of the formulation) is absorbed in less than about 60 seconds, or in less than about 50 seconds, or in less than about 40 seconds, or in less than about 30 seconds, or in less than about 20 seconds, or in less than about 10 seconds, or in less than about 8 seconds, or in less than about 6 seconds, or in less than about 5 seconds, or in less than about 4 seconds, or in less than about 3 seconds, or in less than about 2 seconds, or in less than about 1 second, or any time period in between. In some embodiments, the great majority of the formulation (e.g., more than about 75% of the formulation) is absorbed in less than about 60 seconds, or in less than about 50 seconds, or in less than about 40 seconds, or in less than about 30 seconds, or in less than about 20 seconds, or in less than about 10 seconds, or in less than about 8 seconds, or in less than about 6 seconds, or in less than about 5 seconds, or in less than about 4 seconds, or in less than about 3 seconds, or in less than about 2 seconds, or in less than about 1 second, or any time period in between.

In some embodiments, the foam or foamable formulations described herein facilitate easy treatment due to a combination of at least the following characteristics: the formulations are freely spreadable, rapidly absorbable, and low density. Other desirable characteristics may include, but are not limited to:

Uniformity: The foam or foamable formulation should be formulated so that it is and can remain uniform or substantially uniform over time. This property may be of particular importance when the product is intended to be a pharmaceutical product with a shelf life of about one year, or about 15 months, or about 18 months, or about two years. In some embodiments, the foamable formulation is shaken before use and is readily re-homogenized upon shaking so the foamable formulation is uniform when dispensed.

Flowability: The foamable formulation, when placed in an aerosol container and pressurized, should be flowable such that it can be expelled through the canister valve. The foamable formulation should preferably also be shakable inside the container. These requirements create a formulation challenge, because low or non-viscous flowable and shakable compositions are prone to undergo phase separation or precipitation.

Quality: Upon release from the can, the foamable formulation should generate a foam of good or excellent quality having low density and small bubble size.

Stability/Breakability: The balance between stability and breakability of the foam coming out of the container is very delicate: on one hand the foam should preferably not be “quick breaking”, i.e., it should at least have short term stability upon release from the pressurized container and not break as a result of exposure to skin temperature; by short term stability is meant that upon exposure to a temperature of 36° C., the foam has a collapse time of about 60 seconds, or more than about 60 seconds, or more than about 90 seconds, or more than about 120 seconds, or more than about 150 seconds, or more than about 180 seconds, or more than about 300 seconds, and on the other hand, the foam should be “breakable”, i.e., it should spread easily, break down and absorb into the skin or membrane upon application of mild shear force.

Skin Feeling: To ensure patient compliance the skin feeling after application should be pleasant, and greasy or waxy residues should be minimal.

Non-irritating: The above requirements should be achieved with the awareness that formulation excipients, especially surfactants, can be irritating, and should preferably be eliminated from the foam or foamable formulation or reduced as much as possible.

In some embodiments, a foamable or foam compositions described herein is capable of retaining a high amount of naftifine in solution even though the composition does not comprise surfactant. Thus, in some embodiments, the compositions described herein do not contain or essentially do not contain a potential source of irritation, i.e., a surfactant.

Delivery: The foam or foamable formulation should also be designed to ensure efficient delivery of a therapeutic agent into the target site of treatment.

Solubility: In some embodiments, the therapeutic agent is soluble in the foam or foamable composition. In some embodiments, the therapeutic agent is naftifine or a pharmaceutically acceptable salt thereof. In some embodiments, the foam or foamable composition is capable of solubilizing at least about 1%, 2%, 3%, 4%, 5%, 6%, 7%, 8%, 9%, or 10% or more by weight of the carrier of naftifine or a pharmaceutically acceptable salt thereof. In some embodiments, the foam or foamable composition is capable of solubilizing about 3%, or about 2.5%, or about 2% by weight of the carrier of naftifine or a pharmaceutically acceptable salt thereof.

In some embodiments, the foam or foamable compositions are capable of providing higher solubility than known gel or cream compositions without requiring surfactants to retain the solubility of naftifine or a pharmaceutically acceptable salt thereof (see e.g., Examples 1 and 2). In some embodiments, the solubility of naftifine or a pharmaceutically acceptable salt thereof in the foam or foamable composition is at least about 1-5 times higher (1, 1.1, 1.2, 1.3, 1.4, 1.5, 2, 2.5, 3, 3.5, 4, 4.5, or 5 times higher or any factor in between) than a gel or cream compositions comprising a surfactant. In some embodiments, the solubility of naftifine or a pharmaceutically acceptable salt thereof in the foam or foamable composition is about 1.5 times higher than gel or cream compositions comprising a surfactant. A surfactant may cause skin irritation. The foamable or foam formulations provided herein have shown surprisingly that naftifine hydrochloride can remain soluble within the formulations at concentrations of about 2% to about 4% naftifine by weight of the carrier in the absence of surfactants (see, e.g., Examples 4 and 5). Accordingly, provided herein are foamable or foam formulations wherein the naftifine hydrochloride is dissolved evenly throughout the entire composition.

In some embodiments, the foamable formulation is clear and transparent when placed under the pressure of the propellant. In some embodiments, the foamable formulation is transparent upon pressurization by the gas propellant.In some embodiments, the foamable formulation is a single phase solution. In some embodiments, the foamable formulation, prior to addition of propellant, is a single phase solution. In some embodiments, the foamable formulation, after the addition of propellant and prior to release, is a single phase solution. In some embodiments, the foamable formulation, prior to addition of propellant, is an emulsion. In some embodiments, the foamable formulation, following addition of propellant, is an emulsion. In some embodiments, the foamable formulation has an acceptable shelf-life of at least about one year, or at least about 15 months, or at least about 18 months, or at least about two years at ambient temperature, or any time period in between.

One of ordinary skill in the art would appreciate that propellants tend to impair stability. Addition of propellant can dilute the formulation and upon release of the formulation the position can reverse. Dissipation of propellant from the formulation on release can increase the formulation concentration. Dilution and concentration increase may affect stability. The foamable formulations herein are surprisingly stable, even in the absence of surfactants and even upon dilution and concentration.

Following accelerated stability studies, the foam formulations provided herein demonstrate desirable texture; they form fine bubbles structures that do not break immediately upon contact with a surface, spread easily on the treated area and absorb quickly. In some embodiments, the bubbles structure is characterized by visual inspection. In some embodiments, the spreading is characterized by dispending and spreading foam onto the skin.

A foamable formulation as provided herein should also be free flowing, to allow it to flow through the aperture of the container, e.g., an aerosol container, and create an acceptable foam.

Upon release from an aerosol container, the foamable formulations provided herein form an expanded breakable foam suitable for topical administration. In some embodiments, the foam is a breakable foam that is thermally stable upon dispensing, for example, as selected by a collapse time of about 60 secs or more (e.g., 50, 60, 70, 80, 90, 100, or more seconds); and yet breaks easily upon application of shear force.

In some embodiments, the shear force is applied by administering a low, a mild or a gentle rub over or onto the dispensed foam. In some embodiments, the shear force applied is sufficient to facilitate the foam's collapse and/or spread. In some embodiments, most or all of the foam collapses and/or spreads upon application of the shear force.

Thus, in some embodiments, there is provided a foamable formulation that provides a good to excellent breakable foam, e.g., using a test discussed below. In some embodiments, the foam displays a collapse time of about 60 seconds or more, or of about 90 seconds or more, or of about 120 seconds or more, or of about 150 seconds or more, or of about 180 seconds or more at 36° C., or any time period in between. In some embodiments, the foam is thermally stable upon exposure to a body surface at normal body temperature.

In some embodiments, the foam displays a collapse time of about 60 seconds or more, or of about 50 seconds or more, or of about 40 seconds or more, or of about 30 seconds or more at 36° C.

Breakable foam that is thermally stable, yet breaks under shear force is desirable. Shear-force breakability of the foam is clearly advantageous over thermally-induced breakability. Thermally sensitive foams start to collapse immediately upon exposure to skin temperature and, therefore, cannot be applied on the hand and afterwards delivered evenly to an afflicted area.

The collapse time of foam represents its tendency to be temperature-sensitive and its ability to be at least stable in the short term so as to allow a user sufficient time to comfortably handle and apply the foam to a target area without being rushed and or concerned that it may rapidly collapse, liquefy and or disappear. Collapse time, as an indicator of thermal sensitivity, is examined by dispensing a given quantity of foam and photographing sequentially its appearance with time during incubation at 36° C.

Foam quality may be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of a more stable foam, which does not collapse spontaneously immediately upon discharge from the container. The finer foam structure looks and feels smoother, thus increasing its usability and appeal.

### Exemplary Compositions and Pharmaceutical Formulations

In some embodiments, provided herein are foam or foamable formulations comprising any of the above-described components in any of the above described amounts. In some embodiments, the foam or foamable formulations exemplified herein can be used for any antifungal active agent. In some embodiments, the antifungal agent is an allylamine (e.g., naftifine or terbinafine), a benzyl amine (e.g., butenafine), or other antifungal agents (e.g., amorolfine). Whilst the foam or foamable formulations described herein comprises by way of example, naftifine, the naftifine can be replaced in some embodiments by other allylamines, such as terbinafine. In some embodiments, the allylamine may be a combination of two or more allylamines, such as, terbinafine and naftifine. In some embodiments, the allylamine can be used in a natural form or in a crystalline form. In some embodiments, the allylamine can be in the form of a salt. In some embodiments, the allylamine can be an acid salt. In some embodiments, the allylamine can be a hydrochloride salt.

In some embodiments, the allylamine can be used in combination with an antifungal agent. In some embodiments, naftifine can be used in combination with another antifungal agent. In some embodiments, the antifungal agent is a polyene antifungal, such as amphotericin B, candicidin, or nystatin, an azole such as an imidazole, a triazole, or a thiazole or is an echinocandin or other known antifungal agent. Lists of various antifungal agents are provided in US Publication No. 2007/0292355, which is incorporated by reference in its entirety.

In some embodiments, the allylamine can be used in combination with one or more additional active agents.

In some embodiments, the other active agent is selected from the group consisting of a non-steroidal anti-inflammatory agent, a local anesthetic agent, a steroid, a keratolytically active agent, a retinoid, antipsoriasis agent, an antiinfective agent, an azole, a steroid, an antimicrobial agent, an antibiotic, an allergen, an analgesic agent, an antiallergic agent, an antibacterial agent, an antifungal agent, an antihistamine, an antimycotic agent, an antiparasitic agent, an antipruritic agent, an antiviral agent, a calcium channel blocker, such as flunarizine, a fungicide, an insecticide, a vasoconstrictor, a vasodilator, a vitamin, a vitamin B, a vitamin B derivative, a vitamin C, a vitamin C derivative, a vitamin F, a vitamin F derivative, a vitamin K, and a vitamin K derivative.

In some embodiments, the allylamine can be an allylamine derivative or analogue. In some embodiments, two or more antifungal agents selected from the group consisting of naftifine, terbinafine, butenafine, and amorolfine can be used in the formulations provided herein. In one or more some embodiments, the two or more antifungal agents can be used in a natural form or in a crystalline form. In one or more some embodiments, the two or more antifungal agents can be in the form of salts. In one or more embodiments, the two or more antifungal agents can be acid salts, e.g., hydrochloride salts.

The foamable formulations provided herein comprise a carrier and a liquefied or compressed gas propellant. In some embodiments, the foamable formulation is in a pressurized canister or container. The foamable formulations provided herein form a foam upon expulsion from a canister or container.

For example, provided herein are topical foamable formulations comprising a carrier and a liquefied or compressed gas propellant, wherein the carrier comprises:

about 2% to about 4% by weight of the carrier of naftifine hydrochloride;

about 15% to about 35% by weight of the carrier of a C₁₋₆ alkyl alcohol;

about 15% to about 30% by weight of the carrier of an alkylene glycol;

about 1% to about 2% by weight of the carrier of a polymer;

about 0.5% to about 2.0% by weight of the carrier of a long chain fatty alcohol;

a pH adjuster;

about 0.01% to about 0.3% by weight of the carrier of a chelating agent;

water; and

optionally a preservative;

wherein the propellant is present at about 5% to about 15% by weight of the formulation.

In some embodiments, the preservative is about 0% to about 2% by weight of the carrier. In some embodiments, the preservative is about 0.5% to about 2% by weight of the carrier. In some embodiments, it is about 0.5% to about 1.5% by weight of the carrier. In some embodiments, the carrier is substantially preservative free. In some embodiments, the carrier is essentially preservative free. In some embodiments, the carrier is preservative free. In some embodiments, the presence of a preservative is not needed in the hydroalcoholic formulations provided herein.

In some embodiments, a foamable formulation provided herein comprises:

about 2.5% to about 3% by weight of the carrier of naftifine hydrochloride;

about 18% to about 22% by weight of the carrier of a C₁₋₆ alkyl alcohol;

about 15% to about 23% by weight of the carrier of an alkylene glycol;

about 1.5% to about 2% by weight of the carrier of a polymer;

about 0.5% to about 1.5% by weight of the carrier of a long chain fatty alcohol;

a pH adjuster;

about 0.01% to about 0.3% by weight of the carrier of a chelating agent;

water;

optionally a preservative;

and

about 5% to about 10% by weight of the formulation of a liquefied or compressed gas propellant.

In some embodiments, the foamable formulation is substantially preservative free. In some embodiments, the foamable formulation is essentially preservative free. In some embodiments, the foamable formulation is preservative free. In some embodiments, a foamable formulation provided herein comprises:

about 2.5% to about 3% by weight of the carrier of naftifine hydrochloride;

about 18% to about 22% by weight of the carrier of a C₁₋₆ alkyl alcohol;

about 17% to about 21% by weight of the carrier of an alkylene glycol;

about 1.5% to about 2% by weight of the carrier of a polymer;

about 0.5% to about 1.5% by weight of the carrier of a long chain fatty alcohol;

a pH adjuster;

about 0.01% to about 0.3% by weight of the carrier of a chelating agent;

water;

optionally a preservative;

and

about 5% to about 10% by weight of the formulation of a liquefied or compressed gas propellant.

In some embodiments, a foamable formulation provided herein comprises:

about 2.5% to about 3% by weight of the carrier of naftifine hydrochloride;

about 18% to about 22% by weight of the carrier of a C₁₋₆ alkyl alcohol;

about 15% to about 20% by weight of the carrier of an alkylene glycol;

about 1.5% to about 2% by weight of the carrier of a polymer;

about 0.5% to about 1.5% by weight of the carrier of a long chain fatty alcohol;

a pH adjuster;

about 0.01% to about 0.3% by weight of the carrier of a chelating agent;

water;

optionally a preservative; and

about 5% to about 10% by weight of the formulation of a liquefied or compressed gas propellant.

In the above examples, alternative ranges are provided for the alkylene glycol. Likewise, alternative ranges for the other ingredients may be substituted, and where appropriate making some adjustments to the formulation as would be appreciated by one skilled in the art. For example, the C₁₋₆ alkyl alcohol may be present at about 15% to about 25% by weight of the carrier; or at about 16% to about 24% by weight of the carrier; or at about 17% to about 23% by weight of the carrier; and for example the polymer may be present at about 0.2% to about 2% by weight of the carrier; or at about 0.3% to about 1.9% by weight of the carrier; or at about 0.4% to about 1.8% by weight of the carrier; or at about 0.5% to about 3% by weight of the carrier; or at about 0.6% to about 2.6% by weight of the carrier; or at about 1% to about 2% by weight of the carrier; or at about 1.5% to about 2% by weight of the carrier; or at about 0.01% to about 0.2% by weight of the carrier; or at about 0.2% to about 2.2% by weight of the carrier; or at about 0% to about 0.1% by weight of the carrier; and for example the long chain fatty alcohol may be present at about 0.4% to about 2% by weight of the carrier; or at about 0.7% to about 1.8% by weight of the carrier; or at about 0.8% to about 1.6% by weight of the carrier; or at about 0.5% to about 1.5% by weight of the carrier; at about 0.8% to about 1.2% by weight of the carrier; or at about 0.1% to about 2.5% by weight of the carrier; and for example the chelator may be present at about 0.001% to about 0.1% by weight of the carrier; or at about 0.002% to about 0.2% by weight of the carrier; or at about 0.003% to about 0.3% by weight of the carrier; or at about 0.004% to about 0.4% by weight of the carrier; or at about 0.005% to about 0.5% by weight of the carrier; or about 0.01% to about 0.03% by weight of the carrier; or at about 0% to about 0.02% by weight of the carrier; and for example, water can be present at about 50% to about 60% by weight of the carrier, or at about 48% to about 62% by weight of the carrier, or at about 49% to about 59% by weight of the carrier, or at about 50% to about 59% by weight of the carrier, or at about 52% to about 57% by weight of the carrier, or at about 53% to about 56% by weight of the carrier.

Other ranges or amounts may be used as described throughout the specification.

In some embodiments, a foamable formulation provided herein comprises:

about 2.5 to about 3% by weight of the carrier of naftifine hydrochloride;

about 28 to about 32% by weight of the carrier of a C₁₋₆ alkyl alcohol;

about 25 to about 30% by weight of the carrier of an alkylene glycol;

about 1.5 to about 2% by weight of the carrier of a polymer;

about 0.5 to about 2% by weight of the carrier of a long chain fatty alcohol;

a pH adjuster;

about 0.01 to about 0.3% by weight of the carrier of a chelating agent; and

water; and

about 5 to about 10% by weight of the formulation of a liquefied or compressed gas propellant.

Likewise alternative ranges for the ingredients may be substituted, and where appropriate making some adjustments to the formulation as would be appreciated by one skilled in the art. For example, the C₁₋₆ alkyl alcohol may be present at about 25% to about 35% by weight of the carrier; or at about 26% to about 34% by weight of the carrier; or at about 27% to about 33% by weight of the carrier; and for example the alkylene glycol may be present at about 23% to about 34% by weight of the carrier, or at about 24% to about 33% by weight of the carrier; or at about 25% to about 32% by weight of the carrier; and for example the polymer may be present at about 0.2% to about 2% by weight of the carrier; or at about 0.3% to about 1.9% by weight of the carrier; or at about 0.4% to about 1.8% by weight of the carrier; or at about 0.5% to about 3% by weight of the carrier; or at about 0.6% to about 2.6% by weight of the carrier; or at about 1% to about 2% by weight of the carrier; or at about 1.5% to about 2% by weight of the carrier; or at about 0.01% to about 0.2% by weight of the carrier; or at about 0.2% to about 2.2% by weight of the carrier; or at about 0% to about 0.1% by weight of the carrier; and for example the long chain fatty alcohol may be present at about 0.4% to about 2% by weight of the carrier; or at about 0.7% to about 1.8% by weight of the carrier; or at about 0.8% to about 1.6% by weight of the carrier; or at about 0.5% to about 1.5% by weight of the carrier; at about 0.8% to about 1.2% by weight of the carrier; or at about 1% to about 2% by weight of the carrier; at about 1.2% to about 1.8% by weight of the carrier; or at about 0.1% to about 2.5% by weight of the carrier; and for example the chelator may be present at about 0.001% to about 0.1% by weight of the carrier; or at about 0.002% to about 0.2% by weight of the carrier; or at about 0.003% to about 0.3% by weight of the carrier; or at about 0.004% to about 0.4% by weight of the carrier; or at about 0.005% to about 0.5% by weight of the carrier; or about 0.01% to about 0.03% by weight of the carrier; or at about 0% to about 0.02% by weight of the carrier and for example, water can be present at about 30% to about 40% by weight of the carrier, or at about 28% to about 42% by weight of the carrier, or at about 29% to about 39% by weight of the carrier, or at about 30% to about 39% by weight of the carrier, or at about 32% to about 37% by weight of the carrier, or at about 33% to about 36% by weight of the carrier.

Other ranges or amounts may be used as described throughout the specification.

In some embodiments, the alkylene glycol is propylene glycol; the polymer is a hydroxyalkyl cellulose; and the pH adjuster is an amine base. For example, the alkylene glycol is propylene glycol in an amount of about 19% by weight of the carrier or 28.5% by weight of the carrier; the polymer is a hydroxyalkyl cellulose in an amount of about 1.75% by weight of the carrier; and the pH adjuster is an amine base.

In some embodiments, the C₁₋₆ alkyl alcohol is ethanol; and the long chain fatty alcohol is cetostearyl alcohol. For example, the C₁₋₆ alkyl alcohol is ethanol in an amount of about 20% by weight of the carrier or 30% by weight of the carrier; and the long chain fatty alcohol is cetostearyl alcohol in an amount of about 1.0% by weight of the carrier or 1.5% by weight of the carrier.

In some embodiments, the hydroxyalkyl cellulose is hydroxypropyl cellulose; the pH adjuster is trolamine; and the chelating agent is edetate disodium.

In some embodiments, a foamable formulation provided herein includes:

about 2% to about 4% by weight of the carrier of naftifine hydrochloride;

about 15% to about 35% by weight of the carrier of ethanol;

about 15% to about 30% by weight of the carrier of propylene glycol;

about 1% to about 2% by weight of the carrier of hydroxypropyl cellulose;

about 0.5% to about 2.0% by weight of the carrier of cetostearyl alcohol;

trolamine;

about 0.01% to about 0.3% by weight of the carrier of disodium edetate;

water; and

about 5% to about 15% by weight of the formulation of a liquefied or compressed gas propellant.

For example, a foamable formulation as provided herein includes:

about 3% by weight of the carrier of naftifine hydrochloride;

about 20% by weight of the carrier of ethanol;

about 19% by weight of the carrier of propylene glycol;

about 1.75% by weight of the carrier of hydroxypropyl cellulose;

about 1% by weight of the carrier of cetostearyl alcohol;

optionally about 1% by weight of the carrier of benzyl alcohol;

about 0.17% by weight of the carrier of trolamine;

about 0.02% by weight of the carrier of disodium edetate;

about 54.06% to about 55.06% by weight of the carrier of water; and

about 8% by weight of the formulation of AP-70 propellant.

For example, a foamable formulation as provided herein includes:

about 20% by weight of the carrier of ethanol;

about 19% by weight of the carrier of propylene glycol;

about 1.75% by weight of the carrier of hydroxypropyl cellulose;

about 1% by weight of the carrier of cetostearyl alcohol;

optionally about 1% by weight of the carrier of benzyl alcohol;

about 0.14% by weight of the carrier of trolamine;

about 0.02% by weight of the carrier of disodium edetate;

about 58.09 to about 59.09% by weight of the carrier of water; and

about 8% by weight of the formulation of AP-70 propellant.

In some embodiments, a foamable formulation as provided herein includes:

about 2.5% by weight of the carrier of naftifine hydrochloride;

about 20% by weight of the carrier of ethanol;

about 19% by weight of the carrier of propylene glycol;

about 1.75% by weight of the carrier of hydroxypropyl cellulose;

about 1% by weight of the carrier of cetostearyl alcohol;

optionally about 1% by weight of the carrier of benzyl alcohol;

about 0.14% by weight of the carrier of trolamine;

about 0.02% by weight of the carrier of disodium edetate;

about 54.59% to about 55.59% by weight of the carrier of water; and

about 8% by weight of the formulation of AP-70 propellant.

In some embodiments, a foamable formulation as provided herein includes:

about 2.5% by weight of the carrier of naftifine hydrochloride;

about 30% by weight of the carrier of ethanol;

about 28.5% by weight of the carrier of propylene glycol;

about 1.75% by weight of the carrier of hydroxypropyl cellulose;

about 1.5% by weight of the carrier of cetostearyl alcohol;

optionally about 1% by weight of the carrier of benzyl alcohol;

about 0.14% by weight of the carrier of trolamine;

about 0.02% by weight of the carrier of disodium edetate;

about 34.59% to about 35.59% by weight of the carrier of water; and

about 8% by weight of the formulation of AP-70 propellant.

In some embodiments, a foamable formulation as provided herein includes:

about 3% by weight of the carrier of naftifine hydrochloride;

about 30% by weight of the carrier of ethanol;

about 28.5% by weight of the carrier of propylene glycol;

about 1.75% by weight of the carrier of hydroxypropyl cellulose;

about 1.5% by weight of the carrier of cetostearyl alcohol;

optionally about 1% by weight of the carrier of benzyl alcohol;

about 0.17% by weight of the carrier of trolamine;

about 0.02% by weight of the carrier of disodium edetate;

about 34.06% to about 35.06% by weight of the carrier of water; and

about 8% by weight of the formulation of AP-70 propellant.

For example, a foamable formulation as provided herein includes:

3% by weight of the carrier of naftifine hydrochloride;

20% by weight of the carrier of ethanol;

19% by weight of the carrier of propylene glycol;

1.75% by weight of the carrier of hydroxypropyl cellulose;

1% by weight of the carrier of cetostearyl alcohol;

1% by weight of the carrier of benzyl alcohol;

0.17% by weight of the carrier of trolamine;

0.02% by weight of the carrier of disodium edetate;

54.06% by weight of the carrier of water; and

8% by weight of the formulation of AP-70 propellant.

For example, a foamable formulation as provided herein includes:

3% by weight of the carrier of naftifine hydrochloride;

20% by weight of the carrier of ethanol;

19% by weight of the carrier of propylene glycol;

1.75% by weight of the carrier of hydroxypropyl cellulose;

1% by weight of the carrier of cetostearyl alcohol;

0.17% by weight of the carrier of trolamine;

0.02% by weight of the carrier of disodium edetate;

55.06% by weight of the carrier of water; and

8% by weight of the formulation of AP-70 propellant.

In some embodiments, a foamable formulation as provided herein includes:

2.5% by weight of the carrier of naftifine hydrochloride;

20% by weight of the carrier of ethanol;

19% by weight of the carrier of propylene glycol;

1.75% by weight of the carrier of hydroxypropyl cellulose;

1% by weight of the carrier of cetostearyl alcohol;

1% by weight of the carrier of benzyl alcohol;

0.14% by weight of the carrier of trolamine;

0.02% by weight of the carrier of disodium edetate;

54.59% by weight of the carrier of water; and

8% by weight of the formulation of AP-70 propellant.

In some embodiments, a foamable formulation as provided herein includes:

2.5% by weight of the carrier of naftifine hydrochloride;

30% by weight of the carrier of ethanol;

28.5% by weight of the carrier of propylene glycol;

1.75% by weight of the carrier of hydroxypropyl cellulose;

1.5% by weight of the carrier of cetostearyl alcohol;

1% by weight of the carrier of benzyl alcohol;

0.14% by weight of the carrier of trolamine;

0.02% by weight of the carrier of disodium edetate;

34.59% by weight of the carrier of water; and

8% by weight of the formulation of AP-70 propellant.

In some embodiments, a foamable formulation as provided herein includes:

3% by weight of the carrier of naftifine hydrochloride;

30% by weight of the carrier of ethanol;

28.5% by weight of the carrier of propylene glycol;

1.75% by weight of the carrier of hydroxypropyl cellulose;

1.5% by weight of the carrier of cetostearyl alcohol;

1% by weight of the carrier of benzyl alcohol;

0.17% by weight of the carrier of trolamine;

0.02% by weight of the carrier of disodium edetate;

34.06% by weight of the carrier of water; and

8% by weight of the formulation of AP-70 propellant.

In some embodiments, a foamable formulation as provided herein includes:

20% by weight of the carrier of ethanol;

19% by weight of the carrier of propylene glycol;

1.75% by weight of the carrier of hydroxypropyl cellulose;

1% by weight of the carrier of cetostearyl alcohol;

0.14% by weight of the carrier of trolamine;

0.02% by weight of the carrier of disodium edetate;

58.09% by weight of the carrier of water; and

8% by weight of the formulation of AP-70 propellant.

In some embodiments, a foam or foamable formulation as provided herein further comprises a preservative. For example, the preservative may be benzyl alcohol in an amount of about 1% by weight of the carrier. In some embodiments, when benzyl alcohol is present the amount of water is reduced by the amount of benzyl alcohol added. In some embodiments, the amount of glycol may be reduced by the amount of benzyl alcohol added. In some embodiments, the amount of alkyl alcohol may be reduced by the amount of benzyl alcohol added. In some embodiments, two or more of the components of the formulation may be reduced by the amount of benzyl alcohol added. The reduction can be approximately equally or approximately in proportion or otherwise.

In one or more embodiments, the foamable formulation comprises a carrier and a propellant, wherein the carrier comprises water, a C₁₋₆ alkyl alcohol, an alkene glycol and a fatty alcohol. In some embodiments, the carrier further comprises an allylamine in a therapeutically effective amount. In some embodiments, the carrier may further comprise a polymeric agent. In some embodiments, the carrier may further comprise a pH adjuster or base. In some embodiments, the carrier may further comprise a chelating agent. In some embodiments, the carrier may further comprise a preservative. In some embodiments, the alkyl alcohol comprises ethanol. In some embodiments, the alkene glycol comprises propylene glycol. In some embodiments, the fatty alcohol comprises cetyl and/or stearyl alcohol. In some embodiments, the allylamine comprises naftifine hydrochloride. In some embodiments, the polymeric agent, if present, comprises a cellulose derivative, for example, a hydroxypropyl cellulose. In some embodiments, the pH adjuster, if present, comprises trolamine. In some embodiments, the chelating agent, if present, comprises EDTA. In some embodiments, the preservative, if present, comprises benzyl alcohol. Any and all of the aforesaid ingredients may be present in any of the amounts and or ranges described in the specification.

The foamable formulations are stored in a canister or in an aerosol container and upon release expand rapidly to form a foam formulation. In some embodiments, the expansion is immediate. In some embodiments, about 50% expansion occurs in less than about 7 seconds, or less than about 6 seconds, or less than about 5 seconds, or less than about 4 seconds, or less than about 3 seconds, or less than about 2 seconds, or less than about 1 second, or less than about 0.8 seconds, or less than about 0.7 seconds, or less than about 0.6 seconds, or less than about 0.5 seconds; or less than about 0.4 seconds, or less than about 0.3 seconds, or any time period in between. In some embodiments, about 75% expansion occurs in less than about 10 seconds, or less than about 9 seconds, or less than about 8 seconds, or less than about 7 seconds, is less than about 6 seconds, or less than about 5 seconds; or less than about 4 seconds, or less than about 3 seconds, is less than about 2 seconds, or less than about 1 second, or less than about 0.8 seconds, or less than about 0.7 seconds, or less than about 0.6 seconds, or less than about 0.5 seconds, or less than about 0.4 seconds, or less than about 0.3 seconds, or any time period in between. In some embodiments, about 95% expansion occurs in less than about 30 seconds, or less than about 25 seconds, or less than about 20 seconds, or less than about 15 seconds, is less than about 12 seconds, or less than about 11 seconds, or less than 10 seconds, or less than about 9 seconds, or less than about 8 seconds, or less than about 7 seconds, or less than about 6 seconds, or less than about 5 seconds; or less than about 4 seconds, or less than about 3 seconds, or less than about 2 seconds, or less than about 1 second, or any time period in between. In some embodiments, the foam formulation produces a thermally stable and breakable foam. In some embodiments, the foam formulation is thermally stable at about 36° C., (i.e., similar to skin temperature). In some embodiments, the foam formulation is thermally stable at skin temperature. In some embodiments, thermal stability is indicated by measuring a collapse time as described herein.

The following references are further incorporated by reference herein in their entirety and specifically in relation to their disclosure of foamable ingredients, excipients, formulations, methods and technology: U.S. Pat. Nos. 9,167,813 and 9,072,667.

### Methods of Use

Provided herein are methods for treating fungal infections in a subject, the method comprising topically administering to a subject in need thereof a therapeutically effective amount of a foamable formulation or a foam formulation prepared from a foamable formulation provided herein. In some embodiments, the subject is a pediatric subject.

In some embodiments, administration of a provided foam or foamable formulation reduces the number of fungi in or on the subject to which it is administered. For example, Naftifine and pharmaceutically acceptable salts thereof have fungicidal activity against organisms, including but not limited to, dermatophytes, including for example, Trichophyton rubrum, Trichophyton interdigitale, Trichophyton Trichophyton mentagrophytes, Trichophyton megninii, Trichophyton Trichophyton schoenleinii, Trichophyton soudanense, Trichophyton verrucosum, tonsurans, violaceum, Epidermophyton floccosum, Microsporum audouini, Microsporum canis, Microsporum distortum, Microsporum gypseum; nondermatophyte molds including, for example, Scopulariopsis brevicaulis, Fusarium spp., Aspergillus spp., Alternaria, Acremonium, Scytalidinum dimidiatum, and Scytalidinium hyalinum; and Candida spp. including, for example, Candida albicans, and Candida parapsilosis.

In some embodiments, provided herein are methods for treating a condition, disease or disorder in a subject, wherein the condition, disease or disorder is a fungal infection. In some embodiments, the fungal infection is a fungal infection of the skin. In some embodiments, the fungal infection is a fungal infection of the nail. In some embodiments, the fungal infection is a fungal infection of the hair (e.g., the hair follicle or the hair shaft). In some embodiments, the fungal infection is associated with Trichophyton mentagrophytes. In some embodiments, the fungal infection is associated with Microsporum canis. In some embodiments, the fungal infection is associated with Trichophyton tonsurans.

In some embodiments, a foamable or foam formulation as provided herein is administered to treat a fungal infection in a subject by topically administering the composition to the subject in need thereof, e.g., a subject suffering from tinea capitis. In some embodiments, a foamable or foam formulation as provided herein is administered to treat one or more fungal infections of the skin, particularly of the scalp, and/or of the eyebrows, eyelashes, hair, and/or nails. In some embodiments, the method comprises applying a composition disclosed herein to and/or near the impacted skin. In some embodiments, a composition disclosed herein is administered to a patient suffering from a dermatophytosis. In some embodiments, the dermatophytosis is tinea capitis, tinea favosa (infection by Trichophyton schoeieinii), tinea corporis (ringworm of glabrous skin), tinea imbricata (infection by Trichophyton concentricum), tinea cruris (ringworm of the groin), tinea unguium, or onychomycosis (ringworm of the nail), tinea pedis (ringworm of the feet), tinea barbae (ringworm of the beard), and tinea nianuum (ringworm of the hand).

In some embodiments, a composition disclosed herein is administered to a patient suffering from tinea capitis. In some embodiments, a method is disclosed of treating tinea capitis in a patient in need thereof, the method comprising administering to the patient a topical foam formulation prepared from a topical foamable formulation as provided herein.

In some embodiments, provided herein is the use of a foamable or foam formulation as disclosed herein in the manufacture of a medicament for the treatment of a fungal infection of the skin, particularly of the scalp, and/or of the eyebrows, eyelashes, hair, and/or nails. In some embodiments, provided herein is the use of a foamable or foam formulation as disclosed herein in the manufacture of a medicament for the treatment of a dermatophytosis, e.g., tinea capitis. In some embodiments, a foamable or foam formulation is disclosed, wherein the foamable or foam formulation is used in the treatment of a fungal infection, e.g., tinea capitis.

In some embodiments, provided herein are methods of application of one or more foam or foamable formulations described herein on a skin, mucosa, body cavity, hair, hair follicle, nail surface, or any combination thereof. In some embodiments, the surface or a part thereof or an adjacent area is infected (hereinafter “target surface” or “target skin lesion”). In some embodiments, the infection is a microbial infection. In some embodiments, the infection is a fungal, yeast, bacterial, or a viral infection, or any two or more thereof. In some embodiments, the infection is a fungal infection. In some embodiments, the method involves releasing a foam disclosed herein from a canister or aerosol directly onto the target surface. In some embodiments, the method further comprises collapsing and/or spreading the foam onto the target surface. In some embodiments, the collapsed and/or spread foam is absorbed. In some embodiments, the foam is released onto an intermediate surface such as the fingers or hand before being transferred and applied to a target surface.

In some embodiments, provided herein are methods of treating and/or preventing a fungal infection, including for example, one or more of tinea capitis, tinea favosa, tinea barbae, tinea pedis, tinea cruris and tinea corporis, in a mammal comprising administering to a mammal in need thereof a therapeutically effective amount of a foam formulation as provided herein.

In some embodiments, conditions treated by administration of a foam or foamable formulation provided herein include tinea capitis and/or tinea favosa and/or tinea barbae.

Tinea capitis is a superficial fungal infection (dermatophytosis) of the glabrous and hairy skin (scalp, eyebrows, and eyelashes) that involves the hair shaft and follicles. The disease is primarily caused by dermatophytes in the Trichophyton and Microsporum genera, including for example, Microsporum audouini, Microsporum canis, Microsporum Microsporum distortum, Microsporum gypseum, Trichophyton megninii, Trichophyton mentagrophytes, Trichophyton rubrum, Trichophyton schoenleinii, Trichophyton tonsurans, and Trichophyton verrucosum. The clinical presentation is typically a single or multiple patches of hair loss, sometimes with a ‘black dot’ pattern (often with broken-off hairs), that may be accompanied by inflammation, scaling, pustules, and itching.

Tinea favosa can be considered a variety of tinea capitis because it involves the scalp. Tinea favosa is primarily caused by dermatophytes in the Trichophyton and Microsporum genera, including for example, Microsporum gypseum and Trichophyton schoenleinii.

Tinea barbae is a superficial dermatophytosis that is limited to the bearded areas of the face and neck and occurs almost exclusively in older adolescent and adult males. The clinical presentation of tinea barbae includes inflammatory, deep, kerion-like lesions and non-inflammatory superficial patches resembling tinea corporis or bacterial folliculitis. The mechanism that causes tinea barbae is thought to be similar to that of tinea capitis, and is frequently the result of a Trichophyton rubrum (T. rubrum) infection but may also be the result of Trichophyton mentagrophytes var granulosum and Trichophyton verrucosum. Finally Microsporum canis and Trichophyton mentagrophytes var erinacei have been known to cause tinea barbae but are relatively rare.

Tinea capitis, which may be caused by one or more of Microsporum audouini, Microsporum canis, Microsporum Microsporum distortum, Microsporum gypseum, Trichophyton megninii, Trichophyton mentagrophytes, Trichophyton rubrum, Trichophyton schoenleinii, Trichophyton tonsurans, and/or Trichophyton verrucosum, and tinea favosa, which may be caused by one or more of Microsporum gypseum and/or Trichophyton schoenleinii, and tinea barbae, which may be caused by one of more of Trichophyton rubrum (T. rubrum), Trichophyton mentagrophytes var granulosum, Trichophyton verrucosum, Microsporum canis and Trichophyton mentagrophytes var erinacei, are treatable by the administration of the foam formulations provided herein.

Naftifine hydrochloride has fungicidal activity against multiple fungi species. Accordingly, the administration of the provided foam or foamable formulations may treat, for example, conditions associated with or caused by Microsporum audouini, Microsporum canis, Microsporum distortum, Microsporum gypseum, Trichophyton megninii, Trichophyton mentagrophytes var granulosum, Trichophyton mentagrophytes var ermacel, Trichophyton rubrum, Trichophyton schoenleinii, Trichophyton tonsurans, and/or Trichophyton verrucosum.

Naftifine hydrochloride also has some antibacterial activity against multiple bacterial species. Accordingly, administering the provided foamable and/or foam formulations may treat, for example, conditions associated with or caused by S. pyogenes, E. faecalis, P. vulgaris, P. mirabilis, S. agalactiae, S. aureus, P.aeruginosa.

In some embodiments, provided herein are methods of treating and/or preventing a fungal infection of the skin and/or the nails by administering the disclosed foamable and/or foam formulations.

In some embodiments, conditions treated by administration of a provided foam or foamable formulation include superficial fungal infections of the skin that appear on the outer layer of skin and can cause tinea cruris (jock itch), tinea corporis (ringworm), tinea pedis, interdigital tinea pedis, moccasin-type tinea pedis, tinea manuum, tinea versicolor (piyriasis), tinea nigra, cutaneous candidiasis, tinea faciei (facial ringworm), and white and black piedra.

Tinea corporis (body ringworm), tinea cruris (jock itch), and tinea faciei (facial ringworm), may be caused by Epidermophyton floccosum, Microsporum canis, Trichophyton mentagrophytes, T. rubrum, T. tonsurans, T. verrucosum, and/or T. violaceum, and are treatable by the administration of the present foam formulations.

Tinea pedis (athlete's foot) or tinea manuum (fungal infection of the hand), which may be caused Epidermophyton floccosum, Microsporum canis, Trichophyton mentagrophytes, T. rubrum, T. tonsurans, T. verrucosum, and/or T. violaceum, are treatable by the administration of the present foam formulations.

In various embodiments, the foam and/or foamable compositions are administered at dosages and frequencies sufficient to treat (i.e., reduce, ameliorate, or eliminate) any one or more of the fungal infections discussed herein. In some embodiments, the administered dosages of the foam and/or foamable formulations provided herein are capable of significantly reducing or eliminating fungal infection when administered for about 1, 2, 3, 4, 5, 6, 7, 8, 9, 10, 12, or 14 or more consecutive days. In some embodiments, the foam or foamable formulations provided herein are capable of eliminating fungal infection when administered for about 7 consecutive days. In some embodiments, the foam or foamable formulations are capable of significantly reducing or eliminating fungal infection when applied once a day. In other embodiments the foam or foamable formulations are capable of significantly reducing or eliminating fungal infection when applied twice a day. In other embodiments, the foam or foamable formulations are capable of significantly reducing or eliminating fungal infection when applied three times a day or more often. In some embodiments, the foam or foamable formulations are capable of eliminating fungal infection when applied once a day for at least 7 consecutive days. In some embodiments, the foam or foamable formulations are capable of eliminating fungal infection when applied twice a day for at least 7 consecutive days.

In some embodiments, the foam or foamable formulation comprising naftifine described herein eliminates fungal infection more effectively than in the absence of treatment. In some embodiments, the foam or foamable formulation comprising naftifine eliminates fungal infection more effectively than a placebo formulation such as a foam or foamable formulation that comprises the same ingredients as a naftifine foam or foamable formulation but does not comprise naftifine.

In some embodiments, the effectiveness of the foam or foamable formulation may be assessed by comparing hair samples obtained prior to and post treatment with the formulation. For instance, each hair sample may be cultured to determine whether infecting fungus is present in the hair sample (i.e., mycologically positive). For instance, the effectiveness of the formulation may be expressed as a percentage of the number of mycologically positive hairs in a sample obtained after treatment relative to that in a sample obtained prior to treatment, according to the equation below:

% Efficacy=100−(T×100/K)

wherein, T=Positive hair numbers in the sample obtained after the treatment; and K=Positive hair numbers in the sample obtained prior to the treatment.

In some embodiments, the effectiveness of the foam or foamable formulation may be assessed by comparing hair samples obtained from an infected subject treated with the formulation and an infected control subject treated with a placebo or given no treatment. The effectiveness of the formulation may be expressed as percentage of the number of mycologically positive hairs in a sample obtained from a treated subject relative to that in a sample obtained from a control subject, according to the equation below:

% Efficacy=100−(T×100/K)

wherein, T=Positive hair numbers in the sample obtained from the treated subject; and K=Positive hair numbers in the sample obtained from the control subject.

In some embodiments, the effectiveness of the foam or foamable formulation may be assessed by clinically scoring the affected area prior to and post treatment. Clinical assessment of local changes of the infected skin area may be scored as follows:

0=No lesions

1=Few slightly erythematous places on the skin

2=Well-defined redness, swelling with bristling hairs.

3=Large areas or marked redness incrustation, scaling, bald patches, ulcerated in places.

4=Partial damage to the integument and loss of hair

5=Extensive damage to the integument and complete loss of hair at the site of infection.

The effectiveness in these embodiments may be expressed as percentage of the change of clinical scores due to the treatment, as calculated using the equation below:

% Efficacy=100−(T×100/K)

where, T=Score assessed after the treatment; and K=Score assessed prior to the treatment.

In some embodiments, the effectiveness of the foam or foamable formulation may be assessed by clinically scoring the affected area from an infected subject treated with the formulation and an infected control subject treated with a placebo formulation or given no treatment. Clinical assessment of local changes of the infected skin area may be scored as described above.

The effectiveness in this case may be expressed as percentage of the difference between a clinical score assessed in a treated subject and a clinical score in a control subject, as calculated using the equation below:

% Efficacy=100−(T×100/K)

where, T=Score assessed in a treated subject; and K=Score assessed in a control subject.

In some embodiments, the foam or foamable composition significantly removes or eliminates fungal infection with at least about 5, 10, 20, 25, 30, 40, 50, 60 70, 75, 80, 80, 90, 95, 96, 97, 98, or 99% improved efficiency as compared to a placebo treatment and/or no treatment, or as compared to healthy subjects or healthy hair regions of a patient, as assessed by clinical scoring and/or the number of mycologically positive hairs. In some embodiments, the foam or foamable composition eliminates fungal infection with at least about 25% improved efficiency. In some embodiments, the foam or foamable composition eliminates fungal infection with at least about 50% improved efficiency. In some embodiments, the foam or foamable composition eliminates fungal infection with at least about 75% improved efficiency. In some embodiments the foam or foamable composition eliminates fungal infection with at least about 90% improved efficiency. In some embodiments, the foam or foamable composition eliminates fungal infection with at least about 95% improved efficiency. In some embodiments, the fungal infection is associated with Trichophyton mentagrophytes and the foam or foamable composition significantly reduces or eliminates fungal infection with at least about 90% improved efficiency. In some embodiments, the fungal infection is associated with Microsporum canis and the foam or foamable composition significantly reduces or eliminates fungal infection with at least about 92% improved efficiency. In some embodiments, the fungal infection is associated with Trichophyton tonsurans and the foam or foamable composition significantly reduces or eliminates fungal infection with at least about 85% improved efficiency.

Some embodiments, provided herein are methods of treating and/or preventing a bacterial infection of the skin and/or the body.

In some embodiments, conditions treated by administration of a foam or foamable formulation provided herein include tinea corporis and/or tinea pedis.

Cutaneous candidiasis, which may be caused by Candida albicans, may also be treatable by the administration of the present foam formulations. Naftifine or a pharmaceutically acceptable salt thereof have fungicidal activity against multiple organisms. Accordingly, the administration of the present foam or foamable formulations may treat, for example, superficial fungal infections of the skin related to or caused by Epidermophyton floccosum, Microsporum canis, Microsporum gypseum, Trichophyton mentagrophytes, T. interdigitale, T. rubrum, T. soudanense, T. tonsurans, T. verrucosum, T. violaceum, and Candida albicans.

In some embodiments, the present subject matter relates to a method of treating and/or preventing onychomycosis in a subject comprising administering to a subject in need thereof an effective amount of a presently described foam or foamable formulation.

Non-limiting conditions that are treated by the administration of the present foam or foamable formulations include onychomycosis including onychomycosis caused by one or more of dermatophytes, yeasts (candidal onychomycosis), and non-dermatophyte molds.

Onychomycosis is a fungal infection of the nail bed, matrix, and/or or nail plate. It may be caused by any of the 3 main classes of fungi: dermatophytes, yeasts (candidal onychomycosis), and nondermatophyte molds. Dermatophytes are the most common cause of onychomycosis. Onychomycosis caused by non-dermatophyte molds is becoming more common worldwide. Onychomycosis due to Candida is less common. Dermatophytes that can cause onychomycosis include one or more of Trichophyton rubrum, Trichophyton interdigitale, Epidermophyton floccosum, Trichophyton violaceum, Microsporum gypseum, Trichophyton tonsurans, Trichophyton soudanense, and Trichophyton verrucosum, and dermatophyte associated onychomycosis is often also referred to as tinea ungium. Candidal onychomycosis include cutaneous candidisis and mucocutaneous candidiasis, that are caused by one or more Candida species, including for example, Candida albicans and Candida parapsilosis. Non-dermatophyte molds that can cause onychomycosis can include one or more of, for example, Scopulariopsis brevicaulis, Fusarium spp., Aspergillus spp., Alternaria, Acremonium, Scytalidinum dimidiatum, and Scytalidinium hyalinum.

There are four classic types of onychomycosis including the following: distal subungual onychomycosis (DLSO) that is the most common form of onychomycosis, and is usually caused by Trichophyton rubrum and/or Trichophyton interdigitale, which invades the nail bed and the underside of the nail plate; white superficial onychomycosis (WSO) is caused by fungal (e.g., T. mentagrophytes) invasion of the superficial layers of the nail plate to form “white islands” on the plate, nondermatophyte molds cause deep white superficial onychomycosis; proximal subungual onychomycosis (PSO) is fungal penetration of the newly formed nail plate through the proximal nail fold and it is the least common form of onychomycosis in healthy people, but is found more commonly when the patient is immunocompromised; endonyx onychomycosis (EO), and candidal onychomycosis (CO) which is Candida species invasion of the fingernails.

Naftifine, or a pharmaceutically acceptable salt thereof has fungicidal activity against multiple organisms. Accordingly, the administration of the present compositions may treat, for example, conditions, including for example, onychomycosis, related to or caused by one or more dermatophytes, including for example, Trichophyton rubrum, Trichophyton interdigitale, Epidermophyton floccosum, Trichophyton violaceum, Microsporum gypseum, Trichophyton tonsurans, Trichophyton soudanense, and Trichophyton verrucosum; caused by one or more Candida species, including for example, Candida albicans and Candida parapsilosis; and/or caused by one or more molds, including for example, Scopulariopsis brevicaulis, a Fusarium spp., a Aspergillus spp., Alternaria, Acremonium, Scytalidinum dimidiatum, and Scytalidinium hyalinum.

The provided foam or foamable formulations can be topically administered. A therapeutically sufficient amount of the foam formulation can be collapsed and spread by application of a mechanical or shear force to the foam and gentle rubbing onto the affected area and surrounding skin, for example, in an amount sufficient to cover an affected area plus a margin of healthy skin or tissue surrounding the affected area, for example, a margin of about 0.1, 0.2, 0.3, 0.4, 0.5, 1.0 or more inches, or any amount in between. In some embodiments, the provided foam or foamable formulations can be applied not only to the affected area and the surrounding skin but also to larger amounts of healthy areas, in order to prevent proliferation of the fungal infection.

The formulations provided herein can be applied to any body surface, including for example, skin surface (e.g. glabrous skin), scalp, eyebrows, eyelashes, bearded areas, nail surface, nail bed, nail matrix, and nail fold areas. In some embodiments the foam or foamable formulations can be applied to a body cavity and a cavity surface. In some embodiments, the foam or foamable formulations can be applied to an area of hairy skin, e.g. on the scalp, on the face, on the torso, on an arm, on a leg, and can be applied to large areas including the entire scalp.

As detailed in the examples below, various embodiments of the provided foam or foamable formulations have properties that contribute to the observed treatment.

For example, in some embodiments the foam or foamable formulations provided herein may release the active agent (e.g. naftifine hydrochloride) at a higher rate than previously prepared gel and cream formulations such as gel and cream formulations having surfactant in the formulations such as Polysorbate 20 or Polysorbate 60 in place of the long chain fatty alcohol (e.g., cetostearyl alcohol) present in the foam or foamable formulations provided herein. In some embodiments, the release rate of naftifine from the foam or foamable formulation provided herein is higher than a comparable gel or cream formulation. The term “release rate” means the rate at which drug is released from the foam or foamable formulation following application, and may be calculated by determining the slope of a line obtained by plotting the amount of drug released per unit area of applied surface area against the square root of time, as calculated following Equation (2) in Example 5. In some embodiments, the term “release rate” is interchangeably used with the term “slope.” In some embodiments, the slope is calculated based on the released amount of drug during about 10, 20, 30, 40, 50, 60, 90, 120, 150, or 180 minutes after an initial application of a formulation (see e.g., Example 5).

In some embodiments, the release of naftifine from the foam or foamable formulation peaks earlier than a comparable gel or cream formulation. As used herein the term “peaks earlier” means that a maximal drug release occurs or is achieved earlier. In some embodiments, the maximal release of naftifine from the foam or foamble formulation occurs earlier than a comparable gel or cream formaultion.By avoiding surfactant, various embodiments of the disclosed formulations also reduce skin irritation. In some embodiments, the foam or foamable formulations provided herein release at least about 75% of the active agent within 30 minutes of application. For example, at least about 80%, or about 85%, or about 90% of the active agent is released within 30 minutes of application. In some embodiments, the foam or foamable formulations provided herein release at least about 95% of the active agent within 3 hours of application. For example, at least about 97%, or about 98%, or about 99%, or about 100% of the active agent is released within 3 hours of application. In some embodiment, the percentage of the active agent released within a certain amount of time (i.e., the amount released after a defined amount of time, expressed as a percentage) is calculated such that the theoretical maximum percentage (i.e., 100%) corresponds to release of all the drug present initially in the applied formulation.

As illustrated in Examples 1-5, compared to the gel (Example 1.1.) and cream (Example 1.2) products, the amount released from a foam or foamable formulation exhibits a significant increase as compared to a comparable gel or cream. For example, the percentage of the initial active ingredient released from the foam or foamable formulation provided herein is about 9 times higher than the cream product within 30 minutes of application and about 3.3 times higher within 3 hours of application. As compared to the gel formulation, the percentage of the initial active ingredient released from the foam or foamable formulation is about 1.5 times higher within the first 30 minutes and about 1.2 times higher within 3 hours of application. Without being bound by any theory, it is believed that the increased release rate of the active agent can lead to a quicker onset of action by the active ingredient and also create conditions for its more efficient penetration into relevant (i.e., targeted) skin structures, such as hair follicle and shafts and/or the parts of the epidermis between/ around hair orifices.The foam or foamable formulations provided herein exhibit, in various embodiments an increase in the percentage amount (i.e. μg/cm²), and/or rate (i.e. μg/cm²/h^{0.5}) of the active agent delivered to the target site as compared to previously prepared gel and cream formulations (see Example 5). For example, they may have an increased transportation or delivery rate or percentage of the active agent as compared to gel and cream formulations having surfactant in the formulations such as Polysorbate 20 or Polysorbate 60 in place of the long chain fatty alcohol (e.g., cetostearyl alcohol) present in the foam or foamable formulations provided herein. In some embodiments, a foam formulation as provided herein transports about 4 times more active agent within 30 minutes of administration as compared to a naftifine cream formulation. For example, about 4.6 times more active agent is transported. In some embodiments, a foam or foamable formulation as provided herein transports about 1.5 times more active agent within three hours of administration as compared to a naftifine gel formulation. For example, about 1.6 times more active agent is transported.

Without being bound by any theory or mechanism of action, the increased transportation or delivery of the active agent contributes to or affords significant efficacy and accelerated healing effects exhibited by the disclosed foam and foamable compositions. According to some embodiments, the healing effect may be associated with one or more of: restoration of hair growth, restoration of skin to normal, ringworn itching (associated with healing), elimination or reduction in number of ringworn lesions, elimination or reduction in number of pustules lesions, elimination of or reduction in erythema, and reduction in or no desquamation.

In some embodiments, the foam or foamable formulations provided herein are capable of spreading farther across the site of application (e.g., the scalp) as compared to previously prepared gel and cream formulations (e.g., gel or cream formulations having surfactant in the formulations such as

Polysorbate 20 or Polysorbate 60 in place of the long chain fatty alcohol (e.g., cetostearyl alcohol) present in the foam or foamable formulations provided herein). For example, a foam or foamable formulation provided herein spreads about twice as far as the same amount of a naftifine gel or cream formulation. In some embodiments, a provided foam formulation spreads about three times as far; or about four times as far; or about five times as far; or about six times as far; or about ten times as far, or any amount in between, as compared to the same amount of a naftifine gel or cream formulation. Without being bound by any theory, the increased spreading of a provided foam or foamable formulation distributes the active agent to a larger surface area with a lesser quantity of the active agent relative to other formulations. The effectiveness of the foam or foamable formulations, combined with their low density and high spreadability following application, is noteworthy considering that there is no surfactant per se in the formulations. Without being bound by any theory, it had been believed that surfactants were needed in topical formulations delivered to the scalp because surfactants are thought to disrupt the phospholipid bilayer on the scalp leading to greater penetration of the active agent into the skin. Surfactants are also generally associated with increased fluidity and solubility of active agents (see e.g., Som et al., Journal of Pharmacy & Bioallied Sciences, 2012, 4(1):2-9; and Savjani et al., ISBN Phannaceutics, 2012, 195727). Yet surprisingly, the claimed formulations and methods exhibit good spreadability and target tissue penetration without requiring or substantially without requiring surfactants.

Many surfactants are known to be irritating when left on the skin, as they can extract lipids from the skin, thereby damaging skin barrier and exposing the skin to contact with pro-inflammatory factors (see e.g., Dermatitis, Vol. 33(4) 217-225, 11 Apr. 2006, John Wiley & Sons). In some embodiments, the foam or foamable formulations provided herein are surprisingly and unexpectedly stable, even in the absence of surfactants. In some embodiments, the foam or foamable formulations provided herein have shown surprisingly that naftifine hydrochloride can remain soluble within the formulation in the absence of surfactants.

In some embodiments, the above discussion, observations and parameters noted in relation to the active agent (e.g., naftifine hydrochloride), including increased release, improved onset of release, improved transportation, delivery and spreadability, can also apply when the foam or foamable formulations described herein comprise allylamines other than naftifine hydrochloride, such as terbinafine or comprise mixtures of two or more allylamines.

In some embodiments, methods of treating and/or preventing a fungal infection of skin, mucosa, hair, nails, or any combination thereof, include applying a foam or foamable formulation to an area of skin, mucosa, hair, nails, or any combination thereof, having one or more of the following characteristics:

1. good spreadability;

2. low density;

3. breakable on application of a shear force;

4. thermally stable at 36° C.;

5. small bubble size;

and

6. homogenous.

In some embodiments, the foam is additionally graded as E, G, or FG as described herein. In some embodiments, the foam is graded as E or G as described herein. In some embodiments the foam is graded E. In some embodiments, the foamable formulation before addition of propellant has a low viscosity of about or less than 1000cP. In an exemplary embodiment, the foamable formulation before addition of a propellant has a low viscosity of about 500cP.

In various embodiments, the foam or foamable formulations provided herein may be more effective than previously prepared naftifine gel and cream formulations in the treatment of fungal infections such as tinea capitis. For example, as compared to gel and cream formulations having surfactant in the formulations such as Polysorbate 20 in place of the long chain fatty alcohol (e.g., cetostearyl alcohol) present in the foam or foamable formulations provided herein. For example, the foam or foamable formulations may show a greater decrease in the size of the lesions, greater decrease in the number of lesions, and a greater reduction in the overall severity of the infection.

Finally, in some embodiments the provided foam or foamable formulations are anticipated to lead to better patient compliance. For example, the foams described herein are thermally stable, apply easily to the scalp and other surfaces, provide a pleasant and smooth skin feeling after application, and are readily absorbed into glabrous and hairy skin as compared to a gel or cream formulation. The latter may have less optimal aesthetic properties, e.g. be sticky or greasy or stain clothing.

In some embodiments, the provided foam or foamable formulations can be applied in a single, one-time application, once a week, once a bi-week, once a month, from one to four times daily, or from one to three times daily or any frequency in between, for a period of time sufficient to alleviate symptoms or clear the fungal infection. For example, for a period of time of one day, two days, five days, one week, from 1 to 12 weeks or more, from 1 to 10 weeks, from 1 to 8 weeks, from 2 to 12 weeks, from 2 to 10 weeks, from 2 to 8 weeks, from 2 to 6 weeks, from 2 to 4 weeks, from 4 to 12 weeks, from 4 to 10 weeks, from 4 to 8 weeks, from 4 to 6 weeks, from 4 weeks to six months, or any time period in between. The foam or foamable formulations provided herein can be administered, for example, at a frequency of once per day, twice per day, or three times per day. The foam or foamable formulations provided herein can be topically administered once per day for a period of time from 1 week to 8 weeks, from 1 week to 4 weeks, for 1 week, for 2 weeks, for 3 weeks, for 4 weeks, for 5 weeks, for 6 weeks, for 7 weeks, or for 8 weeks.

In some embodiments, after an initial treatment period of a few days of daily application, the foam or foamable formulations may be used less frequently than daily due to a degree of accumulation of naftifine in the targeted skin structures (hair follicles and shafts and the epidermis extending between the hair openings).

The provided foam or foamable formulations can be applied in a therapeutically effective amount, for example, an amount sufficient to cover an affected area plus a margin of healthy skin or tissue surrounding the affected area, for example, a margin of about 0.5 inches. Suitable amounts, for example, per application per treatment or application area or cumulative daily dosage per treatment or application area (for example two applications in a 24 hour period), can include, for example, about 0.05 grams, or about 0.1 grams, or about 0.15 grams, or about 0.2 grams, or about 0.25 grams, or about 0.3 grams, 0.35 grams, or about 0.4 grams, or about 0.45 grams, or about 0.5 grams, or about 0.55 grams, or about 0.6 grams, about 0.65 grams, or about 0.7 grams, or about 0.75 grams, or about 0.8 grams, or about 0.85 grams, or about 0.9 grams, 0.95 grams, or about 1.0 grams, or about 1.1 grams, or about 1.2 grams, or about 1.3 grams, or about 1.4 grams, or about 1.5 grams, or about 1.6 grams, or about 1.7 grams, or about 1.8 grams, or about 1.9 grams, or about 2.0 grams, or about 2.1 grams, or about 2.2 grams, or about 2.3 grams, or about 2.4 grams, or about 2.5 grams; or from about 0.1 grams to about 8 grams; from about 0.2 grams to about 4.5 grams; from about 0.3 grams to about 4 grams; from about 0.4 grams to about 3.5 grams; from about 0.4 grams to about 3 grams; from about 0.4 grams to about 2.5 grams; from about 0.4 grams to about 2 grams; from about 0.4 grams to about 1.5 grams; from about 0.5 grams to about 8 grams; from about 0.5 grams to about 6 grams; from about 0.5 grams to about 5 grams; from about 0.5 grams to about 4.5 grams; from about 0.5 grams to about 4 grams; from about 0.5 grams to about 3.5 grams; from about 0.5 grams to about 3 grams; from about 0.5 grams to about 2.5 grams; from about 0.5 grams to about 2 grams; from about 0.5 grams to about 1.5 grams; from about 0.5 grams to about 1 gram;

from about 1 gram to about 8 grams; from about 1 gram to about 8 grams; from about 1 gram to about 7 grams; from about 1 gram to about 6 grams; from about 1 gram to about 5 grams; from about 1 gram to about 4.5 grams; from about 1 gram to about 4 grams; from about 1 gram to about 3.5 grams; from about 1 gram to about 3 grams; from about 1 gram to about 2.5 grams; from about 1 gram to about 2 grams; from about 1 gram to about 1.5 grams; from about 1.5 grams to about 8 grams; from about 1.5 grams to about 7 grams; from about 1.5 grams to about 6 grams; from about 1.5 grams to about 5 grams; from about 1.5 grams to about 4.5 grams; from about 1.5 grams to about 4 grams; from about 1.5 grams to about 3.5 grams; from about 1.5 grams to about 3 grams; from about 1.5 grams to about 2.5 grams; from about 1.5 grams to about 2 grams; from about 2 grams to about 8 grams; from about 2 grams to about 7 grams; from about 2 grams to about 6 grams; from about 2 grams to about 5 grams; from about 2 grams to about 4.5 grams; from about 2 grams to about 4 grams; from about 2 grams to about 3.5 grams; from about 2 grams to about 3 grams; from about 2 grams to about 2.5 grams; from about 2.5 grams to about 8 grams; from about 2.5 grams to about 7 grams; from about 2.5 grams to about 6 grams; from about 2.5 grams to about 5 grams; from about 2.5 grams to about 4.5 grams; from about 2.5 grams to about 4 grams; from about 2.5 grams to about 3.5 grams; from about 2.5 grams to about 3 grams; from about 3 grams to about 8 grams; from about 3 grams to about 7 grams; from about 3 grams to about 6 grams; from about 3 grams to about 5 grams; from about 3 grams to about 4.5 grams; from about 3 grams to about 4 grams; from about 3 grams to about 3.5 grams; from about 3.5 grams to about 8 grams; from about 3.5 grams to about 7 grams; from about 3.5 grams to about 6 grams; from about 3.5 grams to about 5 grams; from about 3.5 grams to about 4.5 grams; from about 3.5 grams to about 4 grams; from about 4 grams to about 8 grams; from about 4 grams to about 7 grams; from about 4 grams to about 6 grams; from about 4 grams to about 5 grams; from about 4 grams to about 4.5 grams; from about 4.5 grams to about 8 grams; from about 4.5 grams to about 7 grams; from about 4.5 grams to about 6 grams; from about 4.5 grams to about 5 grams; from about 5 grams to about 8 grams; from about 5 grams to about 7 grams; from about 5 grams to about 6 grams; from about 5.5 grams to about 8 grams; from about 5.5 grams to about 7 grams; from about 5.5 grams to about 6 grams; from about 6 grams to about 8 grams; from about 6 grams to about 7 grams; from about 6.5 grams to about 8 grams; from about 6.5 grams to about 7 grams; from about 7 grams to about 8 grams; from about 7.5 grams to about 8 grams; about 0.2 grams; about 0.5 grams; about 1 gram; about 1.5 grams; about 2 grams; about 2.5 grams; about 3 grams, about 3.5 grams; about 4 grams, about 4.5 grams; about 5 grams, about 5.5 grams; about 6 grams, about 6.5 grams; about 7 grams, about 7.5 grams; or about 8 grams, or any amount in between.

In some embodiments, a foam or foamable formulation is applied at a frequency of from one to four times daily, including for example, once daily, twice daily, three times daily, or four times daily, one a daily or weekly basis, or on a monthly or every other month schedule, for a period of time sufficient to alleviate symptoms or clear the fungal infection. For example, for a period of time from 1 to 52 weeks, from 1 to 26 weeks, from 26 to 52 weeks, from 13 to 39 weeks, from 20 to 40 weeks, from 20 to 48 weeks, from 5 to 50 weeks, from 10 to 45 weeks, from 15 to 40 weeks, from 20 to 35 weeks, from 25 to 30 weeks, for about 30 weeks; from 28 weeks to 50 weeks, from 30 week to 48 weeks, from 32 to 46 weeks, from 34 to 44 weeks, from 36 to 42 weeks, from 38 to 40 weeks, from 2 to 24 weeks, from 2 to 22 weeks, from 2 to 20 weeks, from 2 to 18 weeks, from 2 to 16 weeks, from 2 to 14 weeks, from 2 to 12 weeks, from 2 to 10 weeks, from 2 to 8 weeks, from 2 to 6 weeks, from 2 to 4 weeks, from 10 to 48 weeks, from 12 to 48 weeks, from 14 to 48 weeks, from 16 to 48 weeks, from 18 to 48 weeks, from 20 to 48 weeks, from 22 weeks to 48 weeks, from 24 week to 48 weeks, from 26 to 48 weeks, from 28 to 48 weeks, from 30 to 48 weeks, from 32 to 48 weeks, from 34 to 48 weeks, from 34 to 48 weeks, from 36 to 48 weeks, from 38 to 48 weeks, from 40 to 48 weeks, from 42 to 48 weeks, from 44 to 48 weeks, from 46 to 48 weeks, for 1 weeks, for 2 weeks, for 4 weeks, for 6 weeks, for 8 weeks, for 10 weeks, for 12 weeks, for 24 weeks, for 26 weeks, for 28 weeks, for 30 weeks, for 32 weeks, for 34 weeks, for 36 weeks, for 38 weeks, for 40 weeks, for 42 weeks, for 44 weeks, for 46 weeks, for 48 weeks, for 50 weeks, for 50 weeks, or for 52 weeks. For example, the provided formulations can be topically administered at a frequency of once per day for a period of time from 1 week to 52 weeks, for example for about from 24 weeks to 48 weeks. In some embodiments, the provided formulations can be topically administered at a frequency of twice per day for a period of time from 1 week to 24 weeks, for example for about from 1 week to 12 weeks, such as for about 1 week to 4 weeks. In some embodiments, the provided formulations can be topically administered at a frequency of thrice per day for a period of time from 1 week to 24 weeks, for example for about from 1 week to 12 weeks, such as for about 1 week to 4 weeks.

### Definitions

The term “carbomer” as used herein refers to a polymer of acrylic acid crosslinked with a polyfunctional compound, hence, a poly (acrylic acid) or polyacrylate.

As used herein, the phrases an “effective amount” or a “therapeutically effective amount” of an active agent or ingredient, or pharmaceutically active agent or ingredient, refer to an amount of the pharmaceutically active agent sufficient enough to reduce, ameliorate, lessen, or partially or fully eliminate one or more symptoms of the disorder or to effect a mycological cure upon administration. Effective amounts of the pharmaceutically active agent will vary with the kind of pharmaceutically active agent chosen, the particular condition or conditions being treated, the severity of the condition, the duration of the treatment, the specific components of the composition being used, and like factors. For example, the presently described compositions can be topically applied in an amount sufficient to cover an affected area plus a margin of healthy skin or tissue surrounding the affected area, for example, a margin of about 0.5 inches, at a frequency, for example, of once a day, for a time period, for example of about two weeks.

The term “prevent,” “preventing,” or “prevention,” as used herein refers to any reduction, no matter how slight, of a subject's predisposition or risk for developing a condition, disease, disorder or symptom thereof. For purposes of prevention, the subject is any subject, and preferably is a subject that is at risk for, or is predisposed to, developing a condition, disease, disorder. The term “prevention” includes either preventing the onset of a clinically evident condition, disease, disorder altogether or preventing the onset of a preclinically evident condition, disease, disorder in individuals at risk. This includes prophylactic treatment of subjects at risk of developing condition, disease, disorder.

As used herein, “subject” or “individual” or “animal” or “patient” or “mammal,” refers to any subject, particularly a mammalian subject, for whom diagnosis prognosis, or therapy is desired, for example, a human.

In some embodiments, a patient is a pediatric patient (i.e. a patient under the age of 16 years at the time of diagnosis or treatment). The term “pediatric” can be further divided into various subpopulations including: neonates (from birth through the first month of life); infants (1 month up to two years of age); children (two years of age up to 12 years of age); and adolescents (12 years of age through 16 years of age (up to, but not including, the seventeenth birthday)). In some embodiments, pediatric can refer to patients up to 12 years of age and adolescents can refer to patients of 12 years of age through to 18 years of age.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or provide improvement to a patient or subject's quality of life.

As used herein “clinical efficacy” refers to a clinically observable beneficial effect in a subject resulting from a treatment, which can be expressed in a measurable parameter of clinical success. In some embodiments the subject is a human and the clinical success is defined as a reduction of one or more of the erythema, desquamation/scaling and papule scores on skin lesions. In some embodiments, erythema, desquamation/scaling and/or papule scores of 0 or 1 are obtained on all skin lesions at the end of therapy or one week thereafter using a 4-grade assessment scale (013 absent, 1—mild, 2—moderate and 3—severe/marked).

As used herein “mycological efficacy” refers to an observable beneficial effect in a subject resulting from a treatment, which can be expressed in a measurable parameter of mycological cure. In some embodiments, it is defined as a reduction of dermatophyte culture obtained from a target skin lesion (most representative of overall severity for all lesions identified prior to treatment) at the end of therapy or one week thereafter. In some embodiments, the subject is a human and a negative potassium hydroxide (KOH) test and a negative dermatophyte culture can be obtained from a target skin lesion (most representative of overall severity for all lesions identified prior to treatment) at the end of therapy or one week thereafter.

As used herein the term “potassium hydroxide (KOH) test” refers to an assay which diagnoses dermatophytes in a patient. The test provides a fungal evaluation of a specimen collected from a dermatophyte. A specimen is collected (e.g. by scraping) from the infected area on the patient (e.g., a sample of skin, nail, or hair). The specimen is then placed onto a microscope slide and is covered with KOH (typically 10% or 20%). The slide is allowed to stand until it becomes clear due to destruction of skin cells, hair, and debris (typically after about five to fifteen minutes). Optionally, and in order to enhance clearing, dimethyl sulfoxide can be added to the slide or the slide may be gently heated. The slide is then visualized under a microscope and fungus evaluation is performed. As used herein the term “dermatophyte culture” refers to a test which identifies the infecting organism in a dermatophyte sample. A specimen taken from a dermatophyte lesion is inoculated into a medium (such as dextrose agar containing) and incubated at 25-30° C. for about two days or more. Fungal growth is investigated under the microscope. According to some embodiments, the term “dermatophyte culture” is interchangeable with the term “mycological/mycologic evaluation”.

Reference to the term “about” is intended to have its usual meaning in the context of pharmaceutical formulations of a reasonable variation around a stated amount that retains the ability to achieve one or more functional effect to substantially the same extent as the stated amount. The term may also refer herein to a value of plus or minus 10% of the stated value; or plus or minus 5%, or plus or minus 1%, or plus or minus 0.5%, or plus or minus 0.1%, or any percentage in between. For example “about 20” may mean or include amounts from 18 up to and including 22, to the extent those amounts achieve one or more functional effect to substantially the same extent as an amount of 20 would.

### EXAMPLES

The invention will be described in greater detail by way of specific examples. The following examples are offered for illustrative purposes, and to exemplify the foam or foamable compositions and methods described herein and are not intended to limit the invention in any manner. Many variations will suggest themselves and are within the full intended scope. Those of skill in the art will readily recognize a variety of non-critical parameters which can be changed or modified to yield essentially the same results.

### General Methods and Materials

Metered Dose: Optionally a metered dosage unit can be utilized to achieve delivery of repeatable measured doses of foam, for example as described in U.S. Patent 8,978,936 entitled “APPARATUS AND METHOD FOR RELEASING A UNIT DOSE OF CONTENT FROM A CONTAINER”, which is incorporated herein by reference in its entirety.

### Materials

Table 1—Exemplary possible ingredients suitable for the production of foamable compositions disclosed herein. Equivalent materials from other manufacturers can also be used satisfactorily.

TABLE 1
Exemplary Possible Ingredients Suitable for the Production of Foamable Compositions

| Chemical Name | Function | Commercial Name | Supplier |
|---|---|---|---|
| Ethanol 95% | Solvent | Ethanol 95% | Biolab |
| Ethanol | Solvent | Ethanol absolute | Biolab |
| Propylene glycol | Solvent | Kollisolv PG | BASF |
| Dimethyl sulfoxide | Solvent | Dimethyl sulfoxide | Sigma |
| Polyethylene glycol | Solvent | Polyethylene glycol | Merck |
| 200 | | 200 | |
| Isopropyl alcohol | Solvent | 2-propanol | J. T. Baker |
| Transcutol | Solvent | Transcutol P | Gatefosse |
| Glycerin | Solvent | Kollisolv ® G99 | BASF |
| Water | Solvent | Purified Water | Teva |
| Glycofurol | Solvent | Tetraglycol | Sigma |
| Hexylene glycol | Solvent | Hexylene glycol | Spectrum |
| Polyethylene glycol | Solvent | Kollisolv ® PEG 400 | BASF |
| 400 | | | |
| Dimethyl isosorbide | Solvent | Arlasolve DMI | Croda |
| Diethyl sebacate | Solvent | Diethyl sebacate | Sigma-Aldrich |
| Isopropyl myristate | Solvent | Crodamol IPM | Croda |
| Oleyl alcohol | Solvent | Kollicream ® OA | BASF |
| Oleic acid | Solvent | Oleic acid | Spectrum |
| Isostearic acid | Solvent | Isostearic acid | Stearinerie Dubois |
| | | | Fils |
| PPG-15 stearyl ether | Solvent | Arlamol ™ PS15 | Croda |
| Olive oil | Solvent | Olive oil | Henry Lamotte |
| | | | GmbH |
| Castor oil | Solvent | Castor oil | Fluka |
| Soybean oil | Solvent | Soybean oil | Henry Lamotte |
| Mineral oil light | Solvent | Mineral oil light | Columbia |
| Octyldodecanol | Solvent | Kollicream ® OD | BASF |
| Diisopropyl adipate | Solvent | Crodamol ™ DA | Croda |
| Capric caprylic | Solvent | Kollisolv ® MCT 70 | BASF |
| triglycerides | | | |
| Sodium pyroglutamic | Solvent | Ajidew N-50 | Ajinomoto |
| acid | | | |
| Cyclomethicone | Solvent | Cyclomethicone 5 | Dow Corning |
| Hydroxypropyl | Polymer | Klucel EF | Hercules |
| cellulose | | | |
| Benzyl alcohol | Preservative | Benzyl alcohol pure, | AppliChem |
| | | NF | |
| Cetostearyl alcohol | Foam adjuvant | Kolliwax CSA 50 | BASF |
| Trolamine | pH adjusting agent | Triethanolamine USP- | Panreac |
| | | NF | |
| Edetate Disodium | Chelating agent | Dissolvine NA2-P | AkzoNobel |
| Naftifine | API (active | Naftifine hydrochloride | Erregierre |
| hydrochloride | pharmaceutical | USP | |
| | ingredient) | | |
| Propellant AP-70 | Propellant | Propellant AP-70 | Aeropress |

Animals: Male albino Harlan-Sprague-Dawley Guinea-Pigs with a body weight of 450-500 g were used in the following studies. Animals were allowed to acclimate for a minimum of 5 days prior to use. Environmental controls for the animal room were set to maintain a temperature of 16 to 22 ° C., a relative humidity of 30 to 70%, and a 12:12 light-dark cycle. All procedures in the Guinea Pig protocols were performed in compliance with the Animal Welfare Act, the Guide for the Care and Use of Laboratory Animals, and the Office of Laboratory Animal Welfare.

Calculations for the release experiments were performed using the HPLC processing software Empower2 and the program EXCEL 2010 from Microsoft Corporation, USA. The calculations were performed using the full number of digits handled by EXCEL 2010.

The test articles (i.e. the foam formulations) were received in pressurized cans. These cans were hand shaken prior to dispensing the test article. To prepare the test article for dosing, the foam was dispensed and allowed to collapse. The test article was dispensed into a 50 mL conical tube, the cap was loosely replaced and allowed to stand until the foam was collapsed. The tube was tapped lightly to aid in collapsing the foam. Once collapsed, the foam was applied to the infected area, in the amount of 0.2 mL/application once or twice daily depending on the treatment group. Untreated control guinea pigs were left untreated.

### Production Under Vacuum

Optionally, the foamable carrier may be produced under nitrogen and under vacuum.

### Canisters Filling and Crimping

Each aerosol canister is filled with the pre-foam formulation (“PFF”, i.e., foamable carrier) and crimped with valve, optionally using a vacuum crimping machine. The process of applying a vacuum will cause most of the oxygen present to be eliminated.

### Pressurizing & Propellant Filling

Pressurizing is carried out using a propellant, e.g. a hydrocarbon gas or gas mixture. Optionally, canisters are then warmed for 30 seconds in a warm bath at 50° C. and well shaken immediately thereafter. Such a process prepares a foamable composition according to certain embodiments as provided herein.

### Tests

By way of non-limiting example the objectives of characterization tests are briefly set out below as would be appreciated by a person of the art.

### Collapse Time

Collapse Time, which is the measure of thermal stability, is examined by dispensing a given quantity of foam and photographing sequentially its appearance with time during incubation at 36° C. The collapse time result is defined as the time when the foam height reaches 50% of its initial height or if the foam has not yet reached 50% of its initial height after say 180 seconds then the collapse time is recorded as being >180. By way of illustration one foam may remain at 100% of its initial height for three minutes, a second foam may reach 90% of its initial height after three minutes, a third foam may reach 70% of its initial height after three minutes, and a fourth foam may reach 51% of its initial height after three minutes, nevertheless in each of these four cases the collapse time is recorded as >180 seconds since for practical purposes for easy application by a patient to a target the majority of the foam remains intact for more than 180 seconds. If the foam for example reaches 50% of its original height after say 100 seconds it would be recorded as having a collapse time of 100 seconds. It is useful for evaluating foam products, which maintain structural stability at skin temperature for at least 1 minute. Foams which are structurally stable on the skin for at least one minute are termed “short term stable” carriers or foams.

Alternatively, a Simple Collapse Time can be assessed by placing a foam sample on the warm fingers of a volunteer and measuring the time it takes to melt on the fingers.

### Density

In this procedure, the foam product is dispensed into vessels (including dishes or tubes) of a known volume and weight. Replicate measurements of the mass of foam filling the vessels are made and the density is calculated. The canister and contents are allowed to reach room temperature. The canister is shaken to mix the contents and then 5-10 mL of the contents is dispensed and discarded. Next, the foam is dispensed into a pre-weighed tube, filling it until excess is extruded Immediately excess foam is leveled off and removed at both ends and the filled tube is weighed on the weighing balance.

### Viscosity

Viscosity is measured with Brookfield LVDV-II+PRO with spindle SC4-25 at ambient temperature and 10, 5 and 1 RPM. Viscosity is usually measured at 10RPM. However, at about the apparent upper limit for the spindle of ˜>50,000CP, the viscosity at 1RPM may be measured, although the figures are of a higher magnitude. Unless otherwise stated viscosity of the pre-foam formulation (PFF) is provided. It is not practical to try and measure the viscosity of the foamable formulation with regular propellants since they have to be stored in sealed pressurized canisters or bottles.

### Chemical Stability

The amount of active agent present is analyzed in foam expelled from various pressurized canisters containing foam formulations using HPLC. Analysis is carried out at zero time and at appropriate time intervals thereafter. The canisters are stored in controlled temperature incubators at one or more of 5° C., at 25° C., at, 40° C. and at 50° C. At appropriate time intervals canisters are removed and the amount of active agent in the foam sample is measured.

### Microscope Size

The light microscope enables observing and measuring particles from few millimeters down to one micron. Light microscope is limited by the visible light wavelength and therefore is useful to measuring size of particles above 800 nanometers and practically from 1 micron (1,000 nanometers). The general appearance of the formulation can also be noted using the light microscope.

### Centrifugation

This test is performed on the pre foam formulation, before addition of propellant. A sample is subjected to centrifugation for 3 minutes at 3000 rpm. The result is determined by visual inspection. If no phase separation; and no creaming; and no sedimentation is observed the formulation is characterized as homogenous or uniform.

### Shakability

Shakability represents the degree to which the user is able to feel / hear the presence of the liquid contents when the filled pressurized canister is shaken. Shaking is with normal mild force without vigorous shaking or excessive force. When the user cannot sense the motion of the contents during shaking the product may be considered to be non-shakable. This property may be of particular importance in cases where shaking is required for affecting proper dispersion of the contents.

Shakability scoring:

Good shakability: 2

Moderate shakability: 1

Not shakable but may still be flowable and allow foam formation of quality: 0

### Example 1

### Formulating Naftifine HCl Gel and Cream Compositions

### Example 1.1

### 2% Naftifine HCl Gel Composition

For use in the experiments provided herein, a naftifine hydrochloride gel formulation from Example 1 of WO 2014/121048 was prepared as described therein. The formulations is shown in Table 2 below:

TABLE 2
Formulation of 2% naftifine hydrochloride gel formulation

| Ingredient | % w/w |
|---|---|
| Purified water USP | 51.06 |
| Polysorbate 20 NF | 5.00 |
| Edetate disodium USP | 0.02 |
| Ethanol | 19.00 |
| Propylene glycol | 20.00 |
| Naftifine hydrochloride USP | 2.00 |
| Hydroxyethyl cellulose | 1.75 |
| Benzyl alcohol | 1.00 |
| Trolamine | 0.17 |
| Total | 100 |

In order to formulate the gel, three separate phases, i.e., an alcohol phase, a solubilizing agent phase and a water phase, were prepared and combined. More specifically, the alcohol phase was prepared by combining the propylene glycol, ethanol, and benzyl alcohol, which were mixed until uniform. Next, naftifine hydrochloride was added and mixed until dissolved. Hydroxyethyl cellulose was then added and mixed until dispersed.

The solubilizing agent phase was prepared by combining purified water, polysorbate 20 and edetate disodium. The ingredients were mixed until dissolved.

Next, the alcohol phase was combined with the solubilizing agent phase and mixed together to obtain a mixed phase. To the mixed phase, ethanol was added, and the resulting product was mixed.

The water phase was prepared by combining trolamine and purified water and mixing until dissolved. Subsequently, the water phase was combined with mixed phases and the product was mixed until hydrated. After that, the product was deaerated with a counter- rotating mixer to obtain a bulk drug product. The bulk drug product may be transferred to a holding tank and/or packaged.

### Example 1.2.

### 2% Naftifine HCl Cream Composition

NAFTIN® Cream, 2%, a commercially available naftifine hydrochloride cream formulation, was used as a comparative composition. The formulation of NAFTIN is shown in Table 3 below:

TABLE 3
Formulation of 2% naftifine hydrochloride cream formulation
Ingredient

Purified water USP
Polysorbate 60
Isopropyl myristate
Stearyl alcohol
Cetyl alcohol
Cetyl esters wax
Natfitine hydrochloride USP
Sorbitan monostearate
Benzyl alcohol
Sodium hydroxide

The NDC code for NAFTIN® Cream, 2% is 45 g NDC 0259-1102-45.

### Example 2

### Solubility of Naftifine HCl in 2% Gel Compositions

As shown above, the gel composition described in WO 2014/121048 contains about 5% of Polysorbate 20, a surfactant. Three additional gel compositions were formulated to assess the effect of the surfactant on the solubility of naftifine HCl. The gel composition from WO 2014/121048 (referred to hereinbelow as “Formulation #1”) and the three additional gel compositions are summarized in Table 4.

TABLE 4
Components of Formulations for Analyzing Naftifine Solubility

| | | Formu- | Formu- | Formu- |
|---|---|---|---|---|
| | | lation #2 | lation #3 | lation #4 |
| | Formu- | (5% propyl- | (2% poly- | (5% hydroxy- |
| Ingredient | lation #1 | ene glycol) | sorbate 20) | ethylcellulose) |
| Purified water | 51.06% | 66.06% | 54.06% | 47.81% |
| Naftifine HCl | 2% | 2% | 2% | 2% |
| Edetate | 0.02% | 0.02% | 0.02% | 0.02% |
| Disodium | | | | |
| Propylene | 20% | 5% | 20% | 20% |
| Glycol | | | | |
| Polysorbate | 5% | 5% | 2% | 5% |
| 20 | | | | |
| Hydroxy- | 1.75% | 1.75% | 1.75% | 5% |
| ethylcellulose | | | | |
| Benzyl | 1% | 1% | 1% | 1% |
| Alcohol | | | | |
| Alcohol 190 | 19% | 19% | 19% | 19% |
| Proof | | | | |
| Trolamine | 0.17% | 0.17% | 0.17% | 0.17% |
| Final pH | 5.52 | 5.0 | 5.5 | 5.6 |

The four formulations were prepared according to the following method: two separate phases, i.e., an active phase and a polysorbate phase were prepared and combined. The active phase was prepared by combining the propylene glycol, ethanol, and benzyl alcohol which were mixed until uniform. Next, naftifine hydrochloride was added and mixed until dissolved. Hydroxyethyl cellulose was then added and mixed until dispersed. The polysorbate phase was prepared by combining purified water, polysorbate 20 and edetate disodium. The ingredients were mixed until dissolved. Next, the active phase was combined with the polysorbate phase and mixed together to obtain a mixed phase. To the mixed phase Trolamine was added, and stirred until the final formulation was formed. Each of the four formulations was prepared such that addition of 0.17% Trolamine was the final step to adjust the pH to a value suitable for dermal administration.

Prior to this final pH adjustment step, all four gels maintained clarity. However, upon addition of Trolamine, Formulation #4, comprising a higher amount of hydroxyl ethylcellulose as compared to other three compositions, became solid after the active phase had been combined with the polysorbate phase. Formulation #2, comprising a lower amount of propylene glycol as compared to three other compositions, became cloudy, suggesting that some portion of previously solubilized naftifine HCl began crashing out from the composition. Interestingly, while Formulation #3 and Formulation #1 are identical, except that Formulation #3 contains a lower amount of Polysorbate 20 as compared to Formulation #1, this difference was sufficient to lower the solubility of naftifine HCl in Formulation #3.

Overall, these comparisons demonstrate that the 2% gel composition requires at least 5% of polysorbate 20 to maintain the solubility of naftifine HCl.

### Example 3

### Solubility of Naftifine Hydrochloride

As discussed above, many surfactants are known to be irritating when left on the skin, as they can extract lipids from the skin, thereby damaging skin barrier and exposing the skin to contact with pro-inflammatory factors (see e.g., Dermatitis, Vol. 33(4) 217-225, 11 Apr. 2006, John Wiley & Sons).

To generate a foam composition that is capable of maintaining the high solubility of naftifine HCl without needing to add in surfactants, the solubility of the API with various excipients was tested. Naftifine hydrochloride was combined with different solvents to identify which solvents are able to solubilize the API. The objective of this study was to determine which solvents can be useful in the development of formulations where the API is soluble. The objective of this study was not to determine the maximal solubility of the API in the tested solvents.

To prepare the mixtures, the API was added at a 1% w/w concentration to each solvent at room temperature under mixing. Solubility at room temperature was then assessed after 24 hours by determining the presence of API crystals using microscopic observation at ×200 magnification under polarized light. The mixture was then heated to about 80° C. for 10 minutes and mixed. Solubility at 80° C. was assessed by visual observation. The mixture was then cooled to room temperature and solubility was assessed after 24 hours by determining the presence of API crystals using microscopic evaluation at ×200 magnification under polarized light. For solvents that were found to solubilize the API, the same procedure was followed with increased concentrations of API.

The composition and results of each of the mixtures involved in this study are detailed in Table 5. The first column in Table 5 relates to the solubility at room temperature before heating to 80° C. The second column relates to the solubility at 80° C. as observed visually. The third column relates to the solubility after the mixture has been heated to 80° C. and cooled down to room temperature.

Solvents which were able to dissolve 8% of API were not further tested as part of this study, and the solubility result was reported as >8%.

TABLE 5
Solubility of naftifine hydrochloride in various solvents

| | | | Max. |
|---|---|---|---|
| | Maximum | Maximum | Solubility |
| | Solubility | Solubility | at RT after |
| Solvent | at RT | at 80° C. | heating at 80° C. |
| Ethanol 95% | ~4% | >8% | >8% |
| Propylene glycol | <1% | >8% | >8% |
| Dimethyl sulfoxide | ~2% | >8% | >8% |
| Polyethylene glycol 200 | <1% | ~6% | ~3% |
| Isopropyl alcohol | <1% | ~2% | ~2% |
| Transcutol | <1% | ~2% | ~2% |
| Glycerin | <1% | ~1% | ~1% |
| Water | <1% | ~1% | ~1% |
| Glycofurol | <1% | ~1% | ~1% |
| Hexylene glycol | <1% | ~1% | ~1% |
| Polyethylene glycol 400 | <1% | <1% | <1% |
| Dimethyl isosorbide | <1% | <1% | <1% |
| Diethyl sebacate | <1% | <1% | <1% |
| Isopropyl myristate | <1% | <1% | <1% |
| Oleyl alcohol | <1% | <1% | <1% |
| Oleic acid | <1% | <1% | <1% |
| Isostearic acid | <1% | <1% | <1% |
| PPG-15 stearyl ether | <1% | <1% | <1% |
| Olive oil | <1% | <1% | <1% |
| Castor oil | <1% | <1% | <1% |
| Soybean oil | <1% | <1% | <1% |
| Mineral oil light | <1% | <1% | <1% |
| Octyldodecanol | <1% | <1% | <1% |
| Diisopropyl adipate | <1% | <1% | <1% |
| Capric caprylic | <1% | <1% | <1% |
| triglycerides | | | |
| Sodium pyroglutamic | <1% | <1% | <1% |
| acid | | | |
| Cyclomethicone | <1% | <1% | <1% |

The above results showed that naftifine hydrochloride did not dissolve in hydrophobic solvents, even under heating. However, it was surprising to discover that naftifine HCl dissolved well in propylene glycol (after heating), ethanol, dimethyl sulfoxide and polyethylene glycol 200 (after heating).

### Example 4

### Preparation of Hydroalcoholic Foam Formulations

As shown Example 3, various solvents were capable of retaining a large amount of naftifine HCl in solution. For instance, the solubility of naftifine HCl in ethanol doubled after heating the solution to 80° C. Surprisingly, the solubility remained higher than 8% even after the solution was cooled to room temperature. Therefore, focusing on maintained solubility after initial heating, the hydroalcoholic foam formulations provided herein were prepared according to the following procedure:

### Step 1

Naftifine hydrochloride and propylene glycol were mixed at 70° C. in a suitable vessel until complete dissolution of the active pharmaceutical ingredient (API) (i.e., naftifine hydrochloride) was achieved, as assessed by microscopic observation. The API remained dissolved in the composition until the end of the manufacturing procedure, as well as in the foam released from the canister. The mixture was cooled to room temperature under mixing.

### Step 2

Ethanol and benzyl alcohol, if present, were added to the mixture of Step 1 at room temperature. Hydroxypropyl cellulose was added at room temperature and mixed until a clear formulation was obtained. Fatty alcohols (e.g., cetostearyl alcohol, cetyl alcohol, stearyl alcohol), if present, were added at room temperature and mixed until a clear formulation was obtained.

### Step 3

In a separate vessel, trolamine and edetate disodium were added to a portion of the water (about 10% of the total formulation) at room temperature and mixed until a uniform suspension was obtained. The remaining water was then added under mixing until a clear solution was obtained.

### Step 4

The water phase of Step 3 was added to the ethanol phase of Step 2 in small portions. The addition was performed under moderate agitation until a uniform composition was reached. At Step 4, as well as in the subsequent steps, the API was in solution (i.e., no API crystals were observed microscopically) and the fatty alcohols were in suspension in the composition. Upon addition of propellant, in Step 6, suspended fatty alcohols dissolved.

### Step 5

The ethanol amount was adjusted by weight as necessary to account for evaporative loss. The amount of ethanol to be added was determined by weighing the vessel containing the formulation and subtracting the vessel's tare weight.

### Step 6

The formulation was filled into aluminum canisters. Each aerosol canister was filled with about 20 grams of formulation and crimped using IWKA Aerofill Dual Pak Crimping Machine with a valve. Aerosol canisters were pressurized with propellant mixture AP-70 by Aeropres composed of propane, butane, and isobutane, using IWKA Aerofill Dual Pak Gas Filling Machine. Each aerosol canister was inspected for closure integrity. Aerosol canisters are equipped with a foaming actuator and a suitable label was attached.

Table 6 shows the composition of hydroalcoholic foam formulations used in the examples provided herein. Compositions A-F do not comprise any surfactant. All compositions retained the high solubility of naftifine HCl.

TABLE 6
Composition of Hydroalcoholic Formulations

| | | | | | | F |
|---|---|---|---|---|---|---|
| Formulation | A | B | C | D | E | (placebo) |
| Ingredient | % w/w | % w/w | % w/w | % w/w | % w/w | % w/w |
| Water, purified | 34.59 | 34.06 | 54.59 | 54.06 | 55.06 | 58.09 |
| Ethanol | 30 | 30 | 20 | 20 | 20 | 20 |
| Propylene | 28.5 | 28.5 | 19 | 19 | 19 | 19 |
| glycol | | | | | | |
| Hydroxypropyl | 1.75 | 1.75 | 1.75 | 1.75 | 1.75 | 1.75 |
| cellulose | | | | | | |
| Benzyl alcohol | 1 | 1 | 1 | 1 | 0 | 0 |
| Cetostearyl | 1.5 | 1.5 | 1 | 1 | 1 | 1 |
| alcohol | | | | | | |
| Trolamine | 0.14 | 0.17 | 0.14 | 0.17 | 0.17 | 0.14 |
| Edetate | 0.02 | 0.02 | 0.02 | 0.02 | 0.02 | 0.02 |
| Disodium | | | | | | |
| Naftifine | 2.5 | 3 | 2.5 | 3 | 3 | 0 |
| hydrochloride | | | | | | |
| Total | 100 | 100 | 100 | 100 | 100 | 100 |
| Propellant | 8 | 8 | 8 | 8 | 8 | 8 |
| AP-70 | | | | | | |

Results

1. Formulations A-F are of excellent quality.

2. Table 6B shows characteristics for Formulation D

TABLE 6B
Characteristics of Formulation D

| Test | Formulation D |
|---|---|
| Density (g/mL) | 0.08 |
| Collapse Time (seconds) | >180 |
| Viscosity | 471 cP |
| API Solubility in Foam | API soluble in foam composition |
| Foam Quality | Excellent |
| Centrifugation | homogenous |

Naftifine was found to be stable after 3m and after 6m at 25° C., 30° C. and 40° C.

3. Table 6C shows characteristics for Formulation E

TABLE 6C
Characteristics of Formulation E

| Test | Formulation E |
|---|---|
| Density (g/mL) | 0.07 |
| Expansion of foam on release | At least 75% foam expansion |
| | occurs within 5 seconds after |
| | release |
| Foam Skin Absorption | Foam absorbs after gentle rubbing |
| | on skin |
| Shakability | 2 |
| API Solubility in Foam | API soluble in foam composition |
| Foam Quality | Excellent |
| Foam Microscopy | No API crystals observed |

Packaging Components

The detailed components of the packaging configuration used in this stability study are provided in Table 7.

TABLE 7
Packaging Components

| Packaging | | Coating of | |
|---|---|---|---|
| Component | Description | Inner Side | Manufacturer |
| Canister | Aluminum canister, | PAM | Nussbaum |
| | 1 inch, 35 × 65 mm | | |
| Valve | 1 inch valve with | Micoflex | Seaquist |
| | dip-tube, | | |
| | ref. 0064286 | | |
| Actuator | Foam spout, | Not Applicable | Seaquist |
| | ref. 1010276 | | |

### Example 5

### Release Experiments

### Example 5.1

### Methods of Assessing Release Characteristics of Formluations

An in vitro release testing apparatus composed of Franz diffusion cells was used. The method was performed in 6-fold for each formulation. The slope of release was compared statistically and acceptance criteria were set based on the statistical method (U-test) described in the SUPAC-SS guideline (see e.g., http://www.gmp-compliance.org/guidemgr/files/1-6-12.PDF).

Based on the obtained drug concentrations and the amount of recipient phase withdrawn at each specific time, the total cumulative released amount of drug was calculated for each diffusion cell and plotted as a function of time. Averages and standard deviations were calculated.

In the calculations, the individual values of Franz cells used in the experiments were considered. Release is theoretically proportional to the square root of time, based on the relation provided by Higuchi et al in the following equation (1):

Q=A\sqrt{D*cₛ*(2c₀−cₛ)}*\sqrt{t} Equation (1)

wherein Q is the released amount of active pharmaceutical ingredient (API) at time t (g); A is the diffusion area (cm²); D is the diffusion coefficient (cm·min⁻¹); cₛ is the saturation solubility of API in the formulation (g·mL⁻¹); C₀ is the API concentration in the matrix (g·mL⁻¹); and t is time (min) (see e.g., Higuchi et al., J. Pharm. Sci. 1961, 50:874-5).

A plot of the amount of drug released per unit area (μg-cm⁻²) against the square root of time yields a straight line, where the slope of the line represents the release rate, a measure that is formulation specific. For each Franz cell, the release rate was calculated from the linear portion of the cumulative amount released, according to equation (2):

$$m = \frac{dQ}{A \sqrt{dt}} \qquad Equation ( 2 )$$

wherein Q is the released amount of API at time t (g); A is the diffusion area (cm²); and t is time (min).

### Example 5.2

### Release Analysis for Intact and Collapsed Foam Formulations

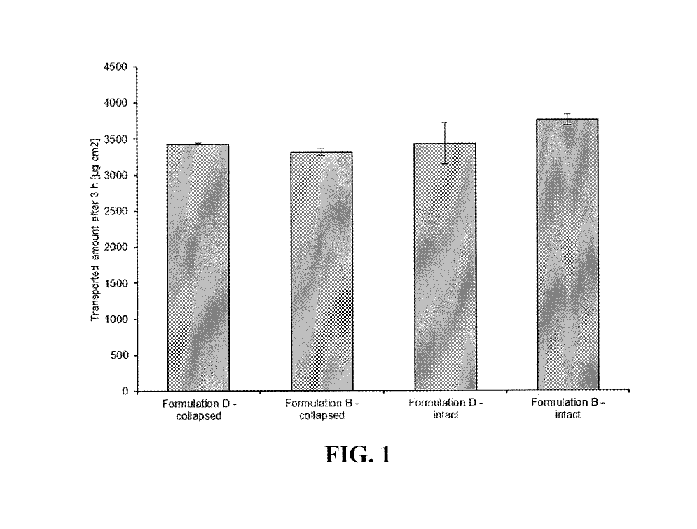

The evaluation of the influence of collapsed and intact foam shows that collapsed foam leads to lower standard deviation than intact foam. This can be explained by the more homogenous contact surface of the collapsed foam to the membrane. When the foam is used as it is sprayed from the aerosol can, the collapsing cannot be controlled. Furthermore, the release experiments showed that the slopes of intact and collapsed foam are different, but the differences between the transported amounts are not relevant. Table 8 and FIG. 1 show the transported amount of naftifine hydrochloride from intact and collapsed foam formulations after 3 hours.

TABLE 8
Transported Amount of Naftifine hydrochloride
from Intact and Collapsed Foam Formulations

| | | | Transported amount after | |
|---|---|---|---|---|
| | | | 3 h (μg · cm⁻²) | |
| | | | | Relative |
| | | | | Standard |
| | | | | Deviation |
| Formulation | Description | State of Foam | Mean | (RSD) (%) |
| B | 3% naftifine | Intact | 3755.53 | 2.00 |
| | hydrochloride, | Collapsed | 3312.01 | 1.30 |
| | hydro-ethanolic | | | |
| | foam | | | |
| D | 3% naftifine | Intact | 3426.95 | 8.24 |
| | hydrochloride, | Collapsed | 3425.71 | 0.68 |
| | hydro-ethanolic | | | |
| | foam | | | |

### Example 5.3

### Comparative Analysis: Foam, Gel, and Cream Formulations

Release characteristics of two foam formulations, one gel (Example 1.1.), and one cream formulation (Example 1.2) were investigated using the Franz cell (i.e., an apparatus comprising an upper chamber, a lower chamber, and a synthetic membrane clamped between the upper and lower chambers) and differences between the formulations were analyzed.

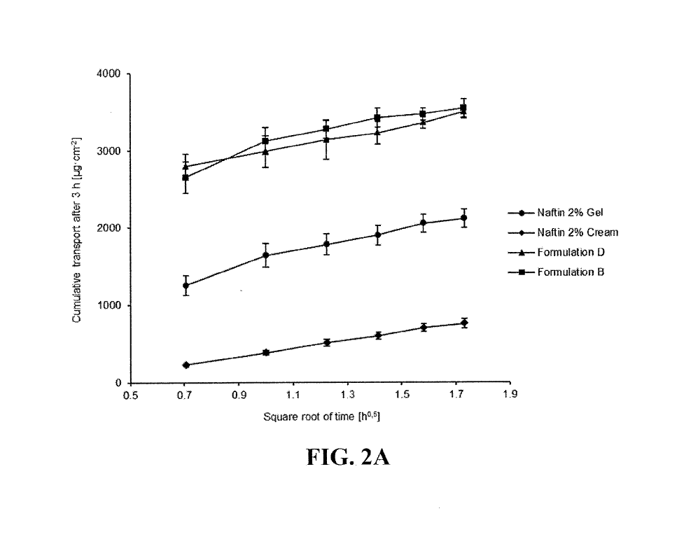

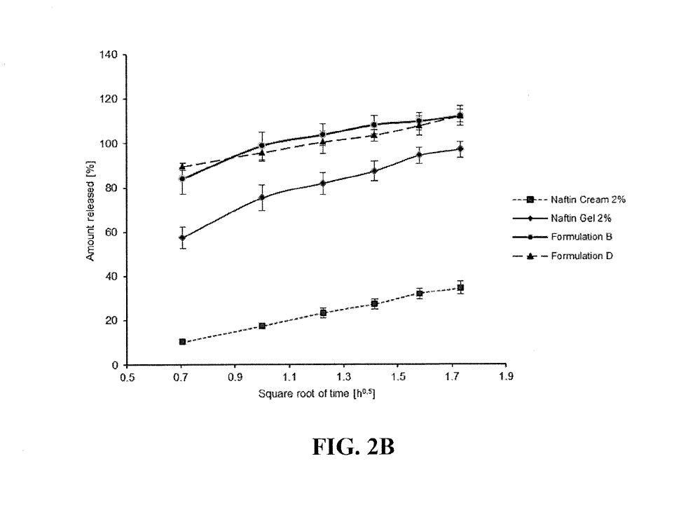

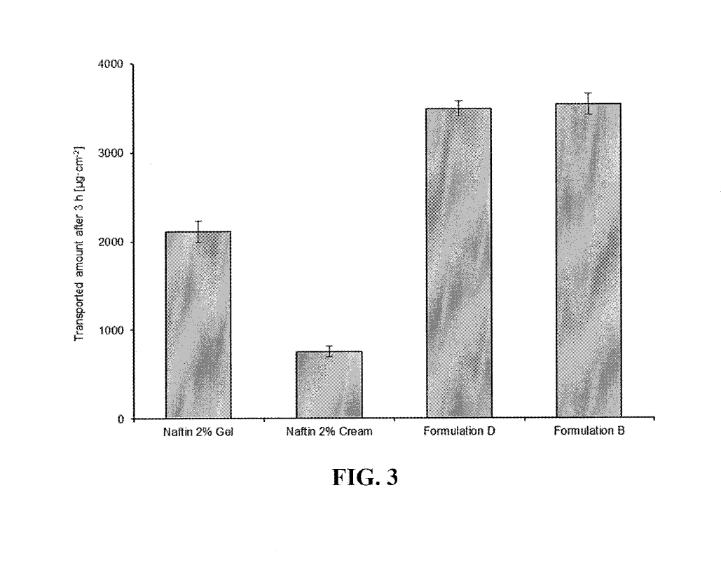

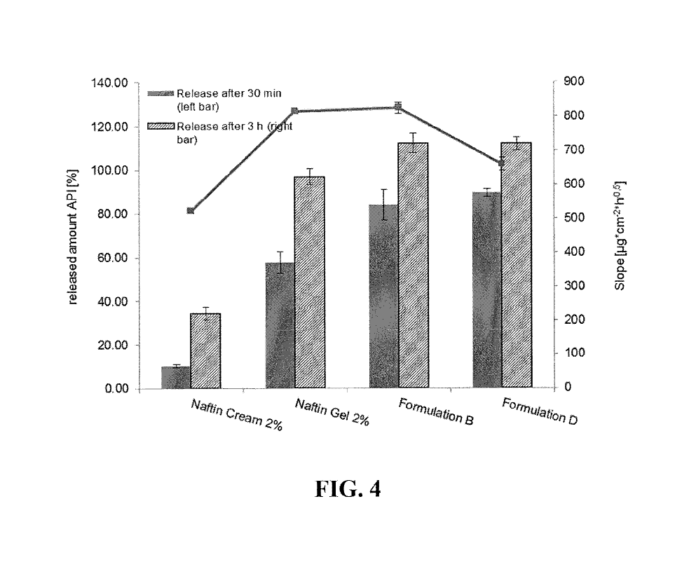

Results of the release experiments of Naftin Cream 2%, Naftin Gel 2%, Formulation B, and Formulation D after 3 hours are shown below in Table 9 and in FIGS. 2A-4. The slope after 3 hours is calculated following Equitation (2) in this example (i.e., a slope of a straight line plotting the amount of drug released per unit area (μg·cm⁻²) against the square root of time). The transported amount after 3 hours indicates the accumulated drug per area (μg·cm⁻²) released after 3 hours, which was measured using a Franz cell. The percentage of the active agent released within a certain time is calculated such that the theoretical amount of drug present in the applied amount of a formulation is set as 100%. Amounts higher than 100% were produced by calculating the cumulative transport. For this calculation, the amount of API removed during each sampling (i.e., a dilution effect) was added to the measured amount. Each step in a measurement or determination is accompanied by deviations. If all these deviations are added, the statistical phenomenon of greater than 100% recovery can occur.

TABLE 9
Results of Release Experiments

| | | | | Transported | | | |
|---|---|---|---|---|---|---|---|
| | | Slope | | Amount | | | |
| | | after 3 h | | after 3 h | | Amount | Amount |
| | | [μg · cm⁻² · h^{-0.5}] | | (μg · cm⁻²) | | Released | Released |
| | | | RSD | | RSD | after 3 h | after 0.5 h |
| Formulation | Description | Mean | (%) | Mean | (%) | (%) | (%) |
| Naftin | 2% naftifine | 523.2 | 8.3 | 753.1 | 8.4 | 34 | 10 |
| Cream 2% | hydrochloride, | | | | | | |
| | cream based | | | | | | |
| Naftin | 2% naftifine | 815.4 | 6.4 | 2111.0 | 5.6 | 97 | 57 |
| Gel 2% | hydrochloride, | | | | | | |
| | gel based | | | | | | |
| B | 3% naftifine | 824.7 | 15.6 | 3542.0 | 3.4 | 112 | 84 |
| | hydrochloride, | | | | | | |
| | hydro-ethanolic | | | | | | |
| | foam | | | | | | |
| D | 3% naftifine | 659.3 | 19.4 | 3490.0 | 2.4 | 112 | 89 |
| | hydrochloride, | | | | | | |
| | hydro-ethanolic | | | | | | |
| | foam | | | | | | |

The released amount increased continuously over time. Naftin Cream 2% has the lowest release rate due to its matrix that contains fatty components such as wax and stearate. The Naftin 2% Gel released approximately three times more API in the same time compared to the 2% Cream formulation, which is mainly attributed to the presumed burst effect (e.g., after 30 minutes, (first sampling) 57% of the applied drug amount is released from the formulation). Additionally, the slope of the release curve (release rate) is greater for the 2% Gel formulation compared to the 2% Cream.

All foams exhibit a “burst effect” similar to the gel formulation. The two hydroethanolic foams released approximately 85% API within the first 30 minutes. The greater the amount of dissolved API in the formulation is, the higher the transport rate due to a steep concentration gradient which leads to a higher diffusion velocity.

Overall, both compositions B and D released a higher amount of naftifine HCl over a 3 hour period (amount released after 3hr (%)) as compared to the gel composition. These data clearly demonstrate that these foam compositions were capable of releasing naftifine HCl faster than the comparable gel compositions. And the foam compositions achieved these superior functional properties without any need of surfactants, components which were required by the gel compositions to maintain the high solubility of naftifine HCl. These data show superiority of foam over gel and cream formulations in that the use of foam formulations leads to higher totally released amount API compared to the gel and cream formulations.

### Example 6

### Topical Treatment of Trichophyton mentagrophytes Dermatophytosis in a Guinea Pig Model

Formulation E was further tested in vivo tinea capitis animal models. T. mentagrophytes ATCC 24953 was obtained from CMM Culture Collection and used as the infecting fungus. The strain was selected because it can cause skin infection and results in inflammatory reaction. Petri dishes were plated with T. mentagrophytes on Potato Dextrose Agar (PDA) and incubated at 30 ° C. for 5-7 days. The colonies were scraped from the plates with sterile normal saline. After washing for three times with sterile saline solution (NaCl, 0.85%) the conidia were re-suspended in the same solution. Ten-fold dilutions of conidia suspension were prepared and counted using a hemacytometer. Working suspension of conidia was prepared at a final concentration of 1×10⁷ Colony Forming Units (CFUs)/100 μL in normal saline.

Each animal was anesthetized with a cocktail of xylazine, and ketamine; 0.2 mL intramuscularly. Using an electric shaver, hair was clipped on left side of the guinea pig back. A closer shave was done with a disposable razor. Using a stencil, a square of 2.5 cm×2.5 cm was marked. The marked skin area was abraded with sterile fine grit sandpaper. A cell suspension containing 1×10⁷ conidia in 100 μL was applied and rubbed thoroughly on the abraded skin.

### Study Procedure

Infected guinea pigs were randomized into the following groups (5 per group); Formulation E foam (once a day); Formulation E (twice a day); and an untreated control. Three days after inoculation, animals were treated topically once or twice daily for a period of seven days, for example, as shown below:

Study Day 1: The animals were infected with dermatophyte (T. mentagrophytes).

Study Day 4: After about 72 hours of infection, the animals were treated with test article, applied once or twice daily for 7 days.

Study Day 10 (treatment Day 7): The last application of the test article to the test area.

Study Day 13: Clinical evaluation of the four quadrants in the marked area was done. Hair samples were obtained from each quadrant. Hairs (10 per quadrant) were planted in Potato Dextrose Agar, and incubated for 48 h for mycological evaluation.

### Hair Root Invasion Test for Mycologic Evaluation

Hair samples were removed with a sterile forceps from four quadrants. Ten hair samples from each quadrant were inoculated onto the corresponding quadrant on the Potato Dextrose agar plate and incubated at 30° C. for 2 days. The fungal growth at the hair root was examined under a stereomicroscope. The effectiveness of a compound in reducing the number of mycologically positive hair samples per treated group is expressed as percentage relative to the untreated control group of animals according to equation (3):

% Efficacy=100−(T×100/K) Equation (3)

wherein, T=Positive hair in the test group; and K=Positive hair in the Untreated control.

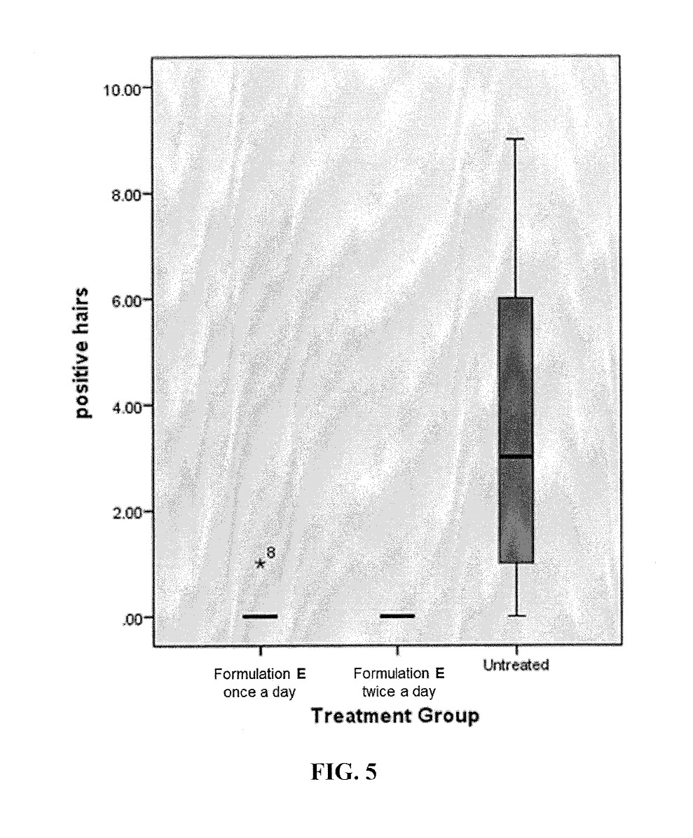

Table 10 and FIG. 5 show the mycological efficacy of each test compound as compared to an untreated control. The controls behaved as expected with the untreated control having the highest average fungal positive hairs. When compared to the untreated controls, percent efficacies for Formulation E (once a day), Formulation E (twice a day), were 98.6% and 100%, respectively. All treatment groups showed significant efficacy when compared to the untreated control (P-values of <0.001).

### Clinical Evaluation

Clinical assessment of local changes of the infected skin area was scored as follows:

0=No lesions

1=Few slightly erythematous places on the skin

2=Well-defined redness, swelling with bristling hairs.

3=Large areas or marked redness incrustation, scaling, bald patches, ulcerated in places.

4=Partial damage to the integument and loss of hair

5=Extensive damage to the integument and complete loss of hair at the site of infection.

Any other local skin reactions were noted. The assessment of clinical evaluation in the change of scores per treatment group is expressed as percentage relative to the untreated control group of animals according to equation (4):

% Efficacy=100−(T×100/K)

where, T=Scores in the test group; and K=Scores in the untreated control

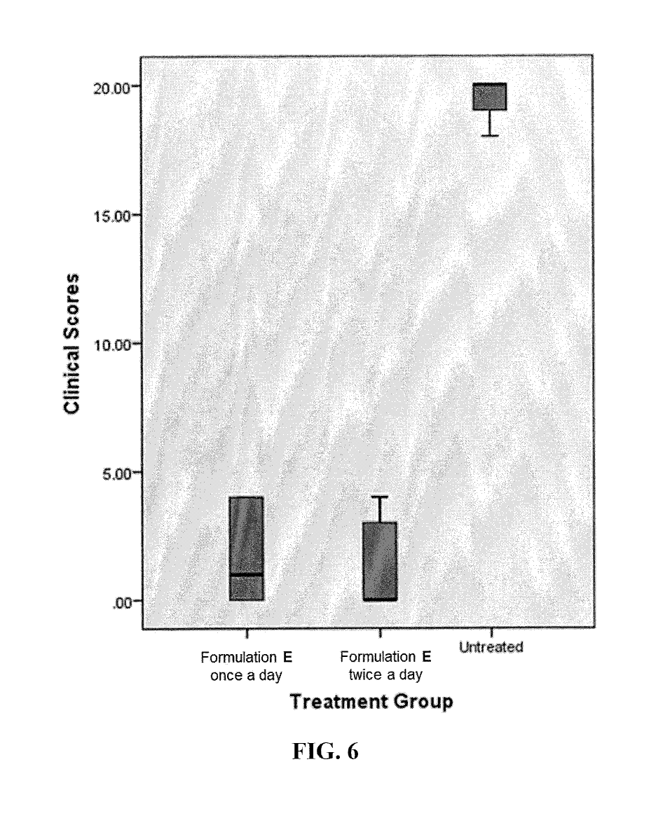

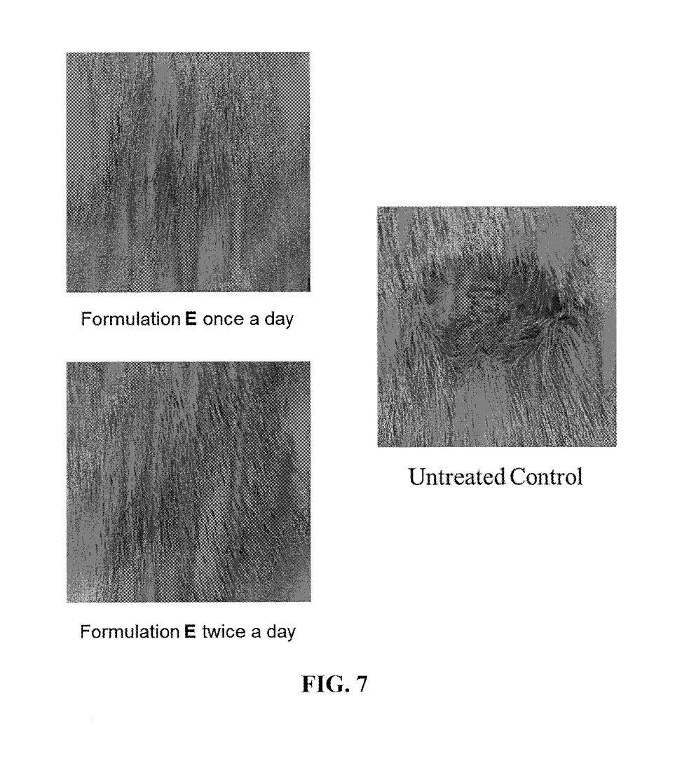

Table 10 and FIGS. 6-7 show the clinical efficacy of each test compound as compared to an untreated control. As expected, the untreated control guinea pigs showed hair loss and ulcerated, scaly skin. When compared to the untreated controls percent efficacies for Formulation E (once a day) and Formulation E (twice a day), were 90.7% and 92.8%, respectively. All treatment groups showed significant efficacy when compared to the untreated controls (P-values of <0.001).

TABLE 10
Summary of the Clinical and Mycological Efficacy
of Treatment Groups Compared to Untreated Control

| | |
|---|---|
| Dose | Percent Efficacy |

| Test Compound | (mg/kg) | Clinical | Mycological |
|---|---|---|---|
| Formulation E | Once a day | 90.7* | 98.6* |
| | Twice a day | 92.8* | 100* |
| Untreated Control | | 0 | 0 |

*P-value of <0.001 when compared to the untreated control

Both treatment groups of Naftifine 3% foam demonstrated significant efficacy both clinically and mycologically when compared to the untreated control. This data clearly demonstrate that naftifine 3% Formulation E foam is highly effective in the treatment of T. mentagrophytes-dermatophytosis in a guinea pig model.

### Example 7

### Topical Treatment of Trichophyton tonsurans Dermatophytosis in a Guinea Pig Model

T. tonsurans MRL #28157 was obtained from the CMM Culture Collection and used as the infecting fungus. Petri dishes were plated with T. tonsurans on Saboraud Dextrose Agar (SDA) and incubated at 30 ° C. for 10 - 13 days. The colonies were scraped from the plates with sterile normal saline. After washing for three times with sterile saline solution (NaCl, 0.85%) the conidia were re-suspended in the same solution. Ten-fold dilutions of conidia suspension were prepared and counted using a hemacytometer. Working suspension of conidia was prepared at the appropriate concentration in CFUs/100 μL in normal saline.

Each animal was anesthetized with a cocktail of xylazine, ketamine and acepromazine; 3:3:1 (by volume); 0.2 mL intramuscularly. Using an electric shaver, hair was clipped on left side of the guinea pig back. A closer shave was done with a disposable razor. Using a stencil, a square of 2.5 cm x 2.5 cm was marked. The marked skin area was abraded with sterile fine grit sandpaper. A cell suspension containing in the inoculum in 100 μL was applied and rubbed thoroughly on the abraded skin.

Guinea pigs were randomized into the following groups (5 per group); T. tonsurans 8×10⁴; T. tonsurans 8×10⁵; and T. tonsurans 8×10⁶ colony forming units (CFUs) Animals were observed daily for signs of infection. Infected guinea pigs were randomized into the following groups (5 per group): Formulation E (once a day); Formulation E (twice a day); Formulation F placebo; and an untreated control. Three days after inoculation, animals were treated topically once or twice daily for a period of seven days, for example, as shown below:

Study Day 1: The animals were infected with dermatophyte (T. tonsurans).

Study Day 4: After about 72 hours of infection, the animals were treated with test article, applied once or twice daily for 7 days.

Study Day 10 (treatment Day 7): The last application of the compounds to the test area.

Study Day 13: Clinical evaluation of the four quadrants in the marked area was done. Procedures for clinical and mycological evaluations are described below.

### Mycologic Evaluation

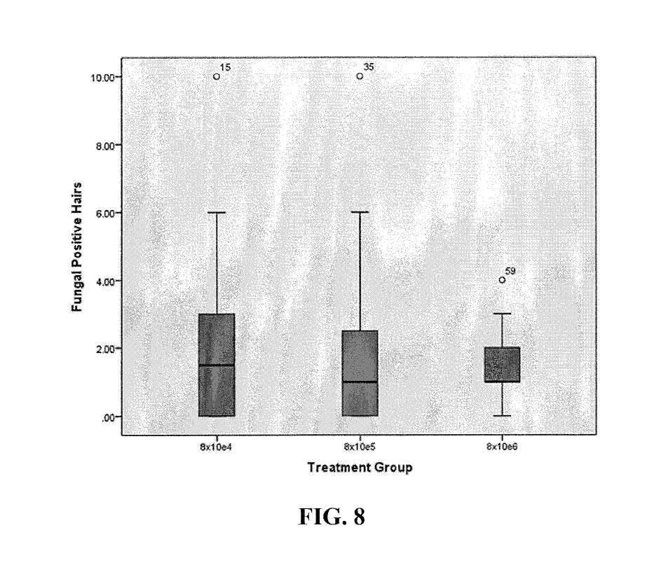

Hair samples were removed with a sterile forceps from four quadrants. Ten hair samples from each quadrant were inoculated onto the corresponding quadrant on the Potato Dextrose agar plate and incubated at 30 ° C. for 2 days. The fungal growth at the hair root was examined under a stereomicroscope. FIG. 8 shows the mycological evaluation of each inocula. The average fungal positive hairs±SD for the T. tonsurans 8×10⁴, 8×10⁵, and 8×10⁶ CFUs were 2.1±2.5, 1.8±2.5, and 1.45±0.99, respectively. There were no significant differences in fungal positive hairs between the inocula (P-values>0.05).

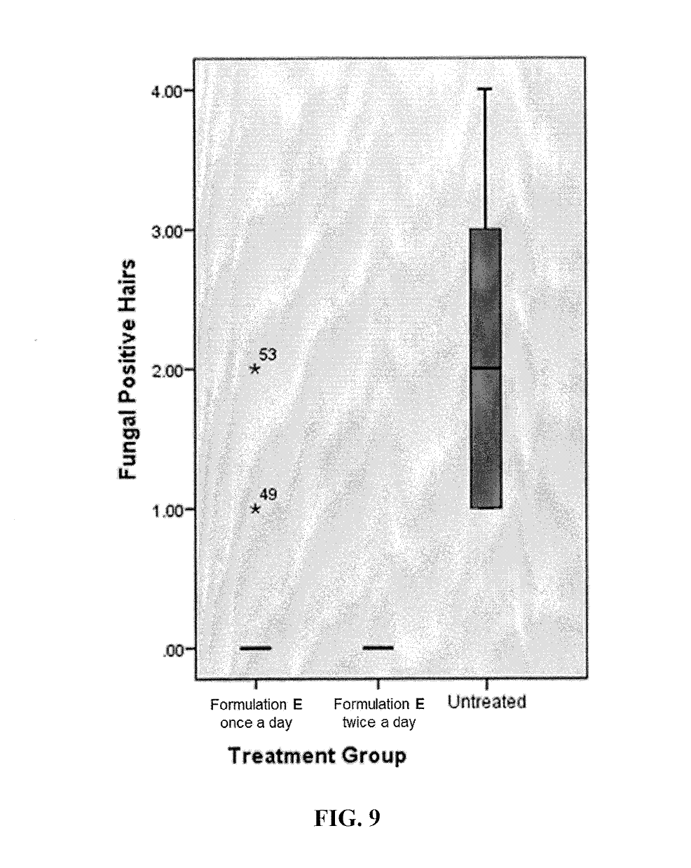

Table 11 and FIG. 9 show the mycological efficacy of each test compound as compared to the untreated control. The control behaved as expected with the untreated control having the highest average fungal positive hairs. When compared to the untreated controls percent efficacies Formulation E (once a day) and Formulation E (twice a day) were 92.7% and 100%, respectively. All treatment groups showed significant efficacy when compared to the untreated control (P-values of <0.001). The placebo (Formulation F)—treated group showed a percent mycological efficacy of 61.0%. However, all other treated groups demonstrated significant mycological efficacy when compared to the placebo-treated control group.

### Clinical Evaluation

Clinical assessment was performed according to the procedures described in Example 6.

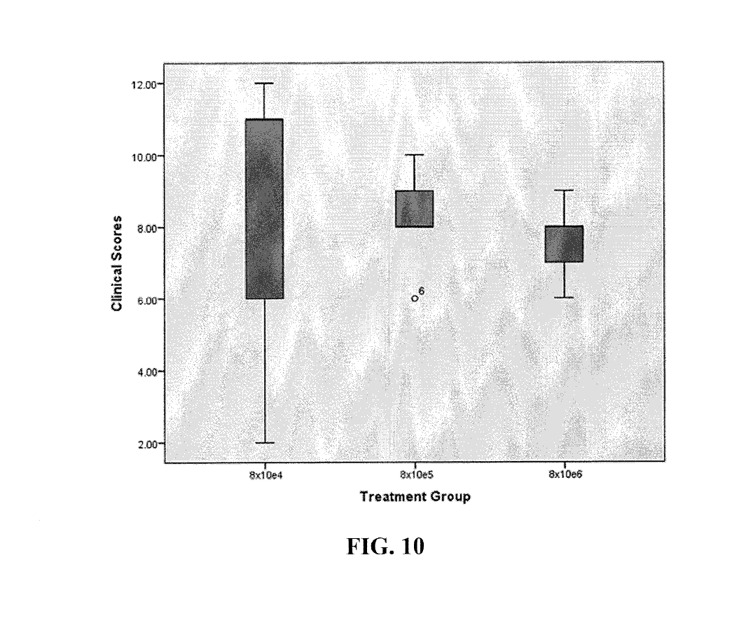

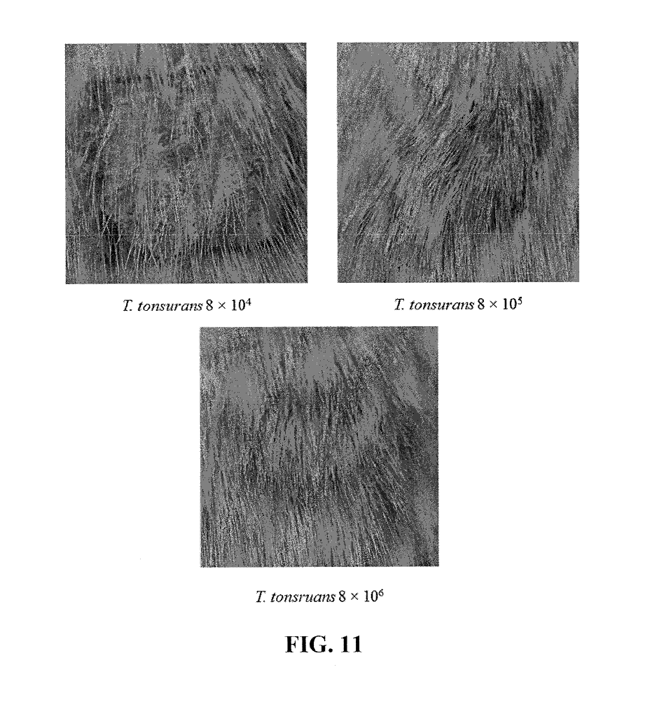

FIGS. 10-11 show the clinical assessment of each inocula. All groups showed scaling and redness. The average clinical score±SD for the T. tonsurans 8×10⁴, 8×10⁵, and 8×10⁶ CFUs were 8.4±4.3, 8.2±1.5, and 7.6±1.1, respectively. There was no significant difference between T. tonsurans infected groups (P-values of >0.05). The clinical symptoms observed in the group infected with T. tonsurans 8×10⁴ were not consistent from animal to animal, while, the 8×10⁵, and 8×10⁶ CFU-infected groups displayed consistent symptoms from animal to animal.

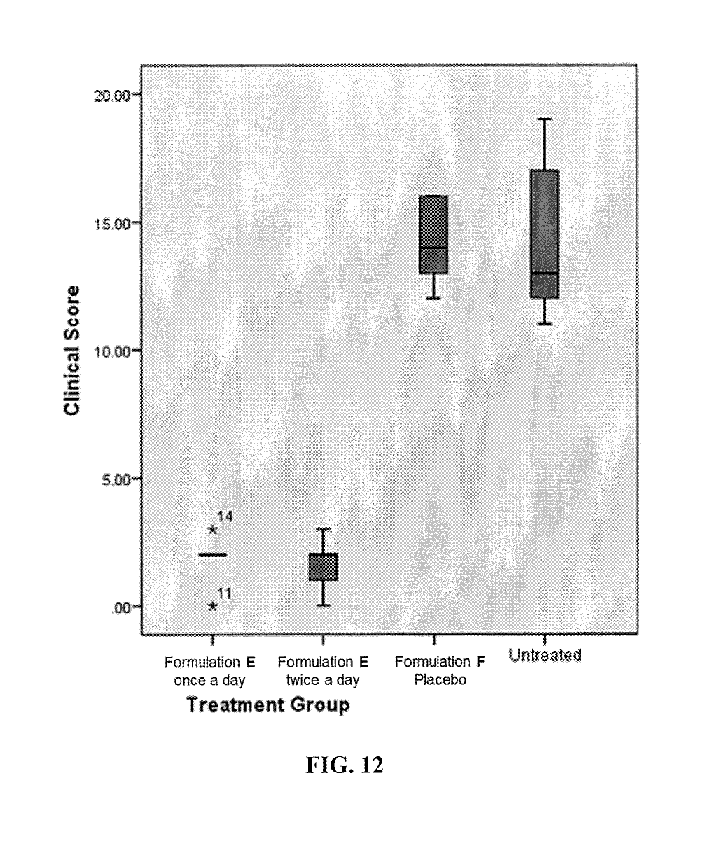

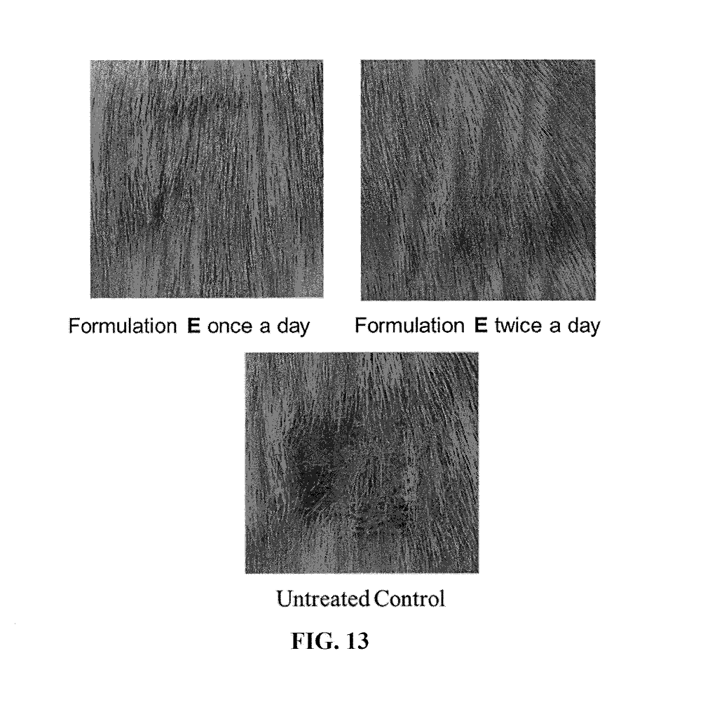

Table 11 and FIGS. 12-13 show the clinical efficacy of each test compound as compared to the untreated control. As expected, the untreated control guinea pigs showed hair loss and ulcerated, scaly skin. When compared to the untreated control, percent efficacies for Formulation E (once a day) and Formulation E (twice a day) were 87.5% and 88.9%, respectively. All treatment groups showed significant efficacy when compared to the untreated controls (P-values of <0.001). The Formulation E once and twice a day—treated groups also showed significant efficacy—treated groups (P-values of 0.001). The placebo (Formulation F)—treated group showed little clinical effect with a percent clinical efficacy of 1.4%. All treated groups demonstrated significant clinical efficacy when compared to the placebo-treated control group (P-values of <0.001).

TABLE 11
Summary of the Clinical and Mycological Efficacy of
Treatment Groups as Compared to the Untreated Control

Percent Efficacy

| Test Compounds | Dose | Clinical | Mycological |
|---|---|---|---|
| Formulation E | Once a day | 87.5^{a,b} | 92.7^{a,b} |
| | Twice a day | 88.9^{a,b} | 100^{a,b} |
| Formulation F | Once a day | 1.4 | 61.0^{a} |
| Untreated Control | | 0 | 0 |

^{a}P-value of <0.05 when compared to the untreated control
^{b}P-value of <0.05 when compared to the placebo control

Both treatment groups of Naftifine 3% foam (given once or twice daily) demonstrated significant efficacy both clinically and mycologically when compared to the untreated and placebo controls. This data clearly demonstrate that Naftifine 3% Formulation E foam is highly effective in the treatment of T. tonsurans-dermatophytosis in a guinea pig model. The placebo Formulation F also exhibited antifungal activity.

### Example 8

### Topical Treatment of Microsporum canis Dermatophytosis in a Guinea Pig Model

M. canis was obtained from the CMM Culture Collection and used as the infecting fungus. Petri dishes were plated with M. canis on Saboraud Dextrose Agar (SDA) and incubated at 30° C. for 10-13 days. The colonies were scraped from the plates with sterile normal saline. After washing for three times with sterile saline solution (NaCl, 0.85%) the conidia were re-suspended in the same solution. Ten-fold dilutions of conidia suspension were prepared and counted using a hemacytometer. Working suspension of conidia was prepared at the appropriate concentration in CFUs/100 μL in normal saline.

Each animal was anesthetized with a cocktail of xylazine, and ketamine; 0.2-0.3 ml intramuscularly. Using an electric shaver, hair was clipped on left side of the guinea pig back. A closer shave was done with a disposable razor. Using a stencil, a square of 2.5 cm×2.5 cm was marked. The marked skin area was abraded with sterile fine grit sandpaper. A cell suspension containing in the inoculum in 100 μL was applied and rubbed thoroughly on the abraded skin.

Guinea pigs were randomized into the following groups (5 per group); M. canis 8×10⁴, M. canis 8×10⁵, and M. canis 8×10⁶ CFUs. Animals were observed daily for signs of infection. Infected guinea pigs were randomized into the following groups (5 per group); Formulation E (once a day), Formulation E (twice a day), and an untreated control.

Three days after inoculation, animals were treated topically once or twice daily for a period of seven days, for example, as shown below:

Study Day 1: The animals were infected with dermatophyte (M. canis).

Study Day 4: After about 72 hours of infection, the animals are treated with test article, applied once or twice daily for 7 days.

Study Day 10 (treatment Day 7): The last application of the compounds to the test area.

Study Day 13: Clinical evaluation of the four quadrants in the marked area is done.

### Mycologic Evaluation

Hair samples were removed with a sterile forceps from four quadrants. Ten hair samples from each quadrant were inoculated onto the corresponding quadrant on the Potato Dextrose agar plate and incubated at 30° C. for 2 days. The fungal growth at the hair root was examined under a stereomicroscope.

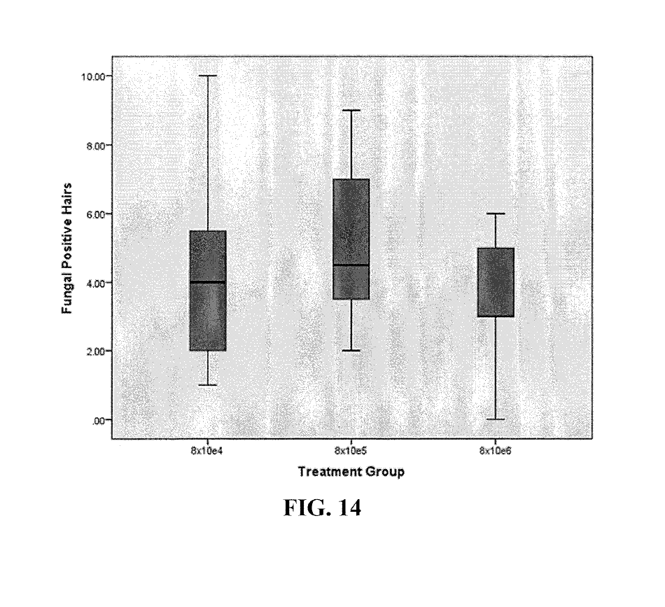

FIG. 14 shows the mycological evaluation of each inocula. The average fungal positive hairs±SD for the M. canis 8×10⁴, 8×10⁵, and 8×10⁶ were 4.1±2.4, 5.1±2.2, and 3.5±1.6, respectively. Animals infected with M. canis 8×10⁵ CFUs showed the highest average fungal positive hairs. However, animals infected with 5×10⁶ CFUs had severe hair loss, therefore samples were not available from each quadrant. There was no statistical difference between inocula (P-value>0.05).

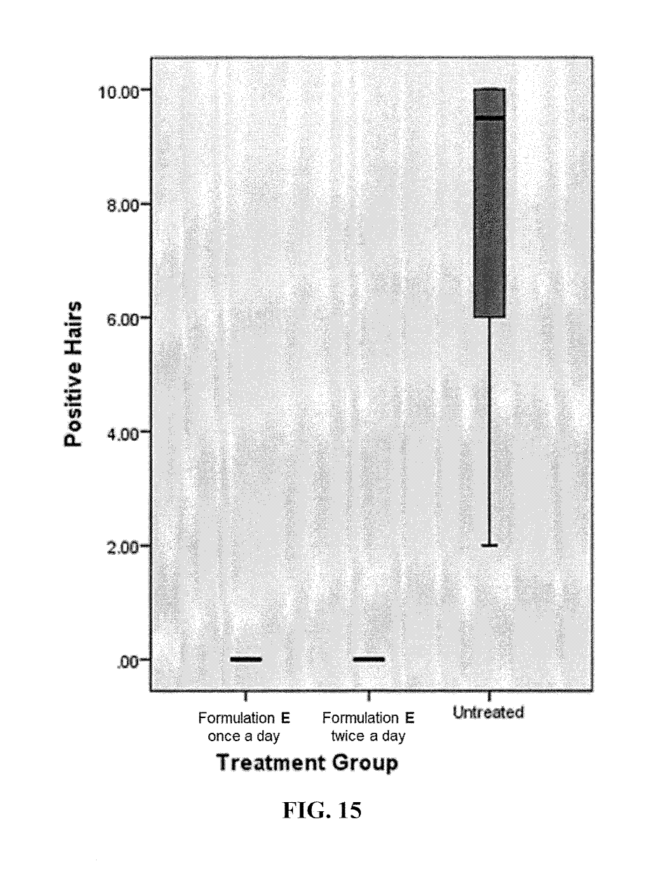

Table 12 and FIG. 15 show the mycological efficacy of each test compound as compared to the untreated control. The controls behaved as expected with the untreated control having the highest average fungal positive hairs. When compared to the untreated controls, percent efficacies for Formulation E (once a day) and Formulation E (twice a day) were 98.1% and 100%, respectively. All treatment groups showed significant efficacy when compared to the untreated control (P-values of <0.001).

### Clinical Evaluation

Clinical assessment was performed according to the procedures described in Example 5.

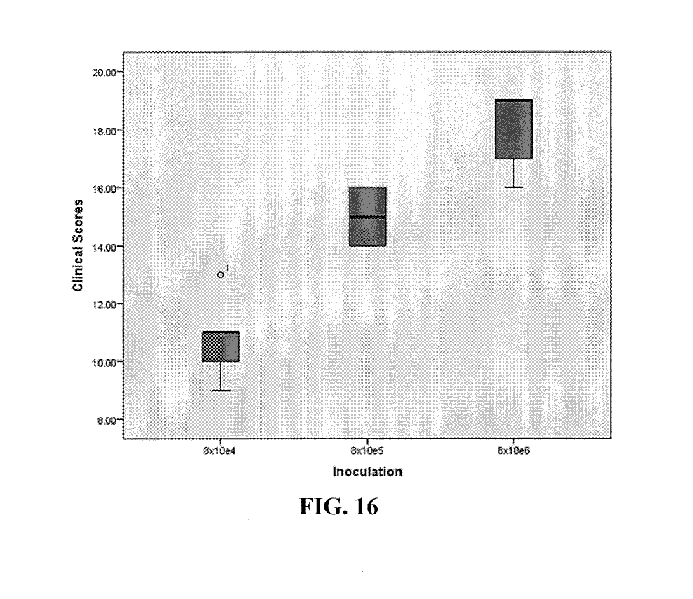

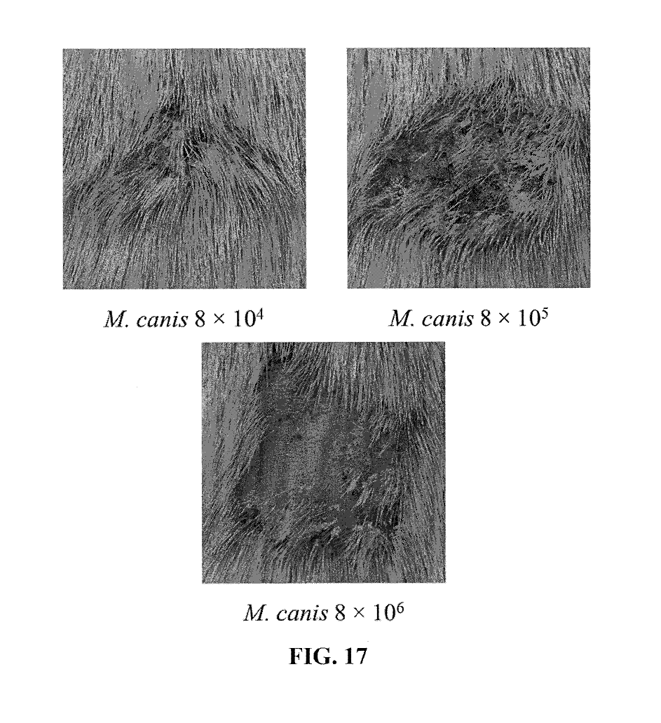

FIGS. 16-17 show the clinical assessment of each inocula. All groups showed scaling and redness and hair loss. The severity of clinical signs increased with the size of the inoculum. The average clinical score±SD for the M. canis 8×10⁴, 8×10⁵, and 8×10⁶ CFUs were 10.8±1.5, 15±1.0, and 18±1.4, respectively . Both the 8×10⁵, and 8×10⁶ CFU inoculum demonstrated significantly higher clinical assessments when compared to the 8×10⁴ (P-values of 0.001 and <0.001, respectively). Also the 8×10⁶ inocula showed a significantly higher clinical score when compared to the 8×10⁵ inocula (P-value of 0.011).

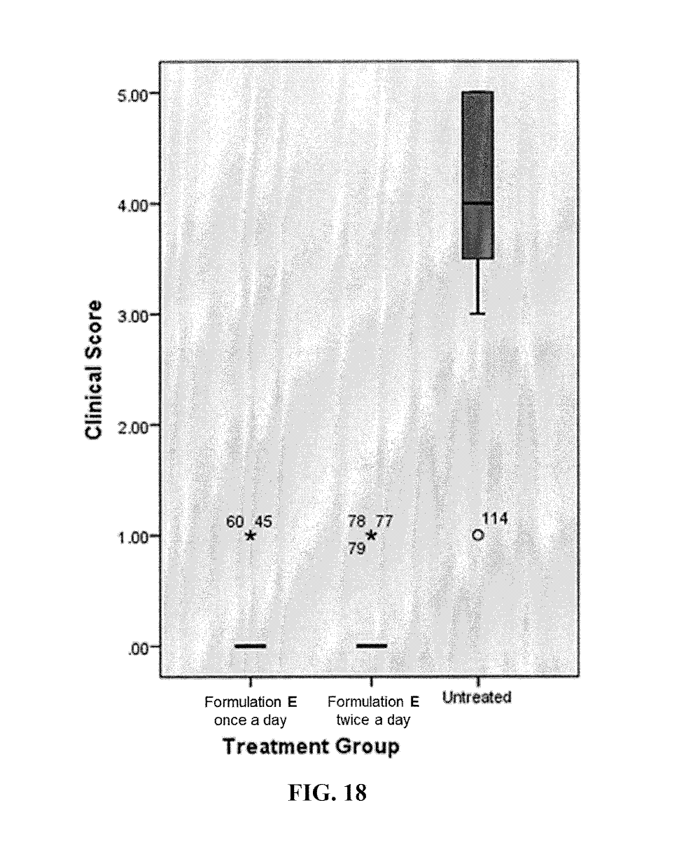

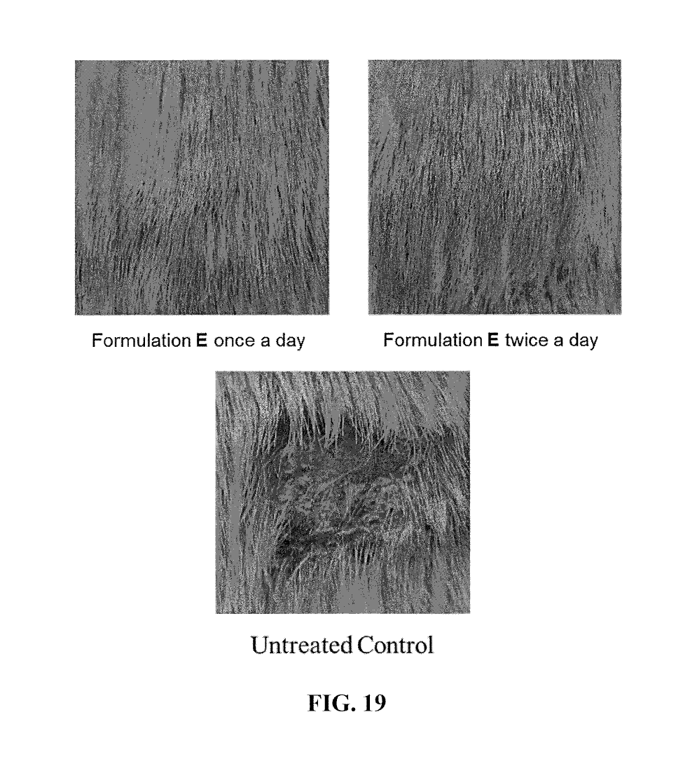

Table 12 and FIGS. 18-19 show the clinical efficacy of each test compound as compared to the untreated control. As expected, the untreated control guinea pigs showed hair loss and ulcerated, scaly skin. When compared to the untreated control, percent efficacies for Formulation E (once a day) and Formulation E (twice a day) were 96.2% and 94.9%, respectively. All treatment groups showed significant efficacy when compared to the untreated controls (P-values of <0.001).

TABLE 12
Summary of the Clinical and Mycological Efficacy of
Treatment Groups Compared to the Untreated Control

Percent Efficacy

| Test Compounds | Dose | Clinical | Mycological |
|---|---|---|---|
| Formulation E | Once a day | 96.2^{a} | 98.1^{a} |
| | Twice a day | 94.9^{a} | 100^{a} |
| Untreated Control | | 0 | 0 |

^{a}P-value of <0.05 when compared to the untreated control

Both treatment groups of Naftifine 3% foam demonstrated significant efficacy both clinically and mycologically when compared to the untreated. This data clearly demonstrate that Naftifine 3% Formulation E foam is highly effective in the treatment of M. canis-dermatophytosis in a guinea pig model.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-77. (canceled)

78. A method of treating tinea capitis in a subject in need thereof, comprising administering topically to an infected area of scalp of the subject a foam prepared from a foamable formulation, wherein the foamable formulation comprises:
(1) a carrier comprising:
(a) about 2% to about 4% by weight of the carrier of naftifine or a pharmaceutically acceptable salt thereof;
(b) about 15% to about 35% by weight of the carrier of a C₁₋₆ alkyl alcohol;
(c) about 15% to about 30% by weight of the carrier of an alkylene glycol;
(d) about 1% to about 2% by weight of the carrier of a polymer;
(e) about 0.5% to about 2% by weight of the carrier of a long chain fatty alcohol;
(f) a pH adjuster;
(g) about 0.01% to about 0.3% by weight of the carrier of a chelating agent; and
(h) water; and
(2) a liquefied or compressed gas propellant, wherein the propellant is present at about 5% to about 15% by weight of the formulation.

79. The method of claim 78, wherein the pharmaceutically acceptable salt form is a hydrochloride salt.

80. The method of claim 78, wherein the C₁₋₆ alkyl alcohol comprises methanol, ethanol, n-propyl alcohol, isopropyl alcohol, n-butyl alcohol, t-butyl alcohol, 2-butanol, iso-butanol, pentanol, hexanol, and/or cyclohexanol.

81. The method of claim 78, wherein the alkylene glycol comprises ethylene glycol, propylene glycol, hexylene glycol, and/or butylene glycol.

82. The method of claim 78, wherein the polymer comprises a methylcellulose, a hydroxypropyl cellulose, a hydroxypropyl methylcellulose, a hydroxyethyl cellulose, a hydroxyl propylmethyl cellulose, a methylhydroxyethylcellulose, a methylhydroxypropylcellulose, a hydroxyethylcarboxymethylcellulose, a carboxymethyl cellulose, a carboxymethylcellulose, and/or a carboxymethylhydroxyethylcellulose.

83. The method of claim 78, wherein the long chain fatty alcohol comprises myristyl alcohol, arachidyl alcohol, behenyl alcohol, cetyl alcohol, and/or stearyl alcohol.

84. The method of claim 78, wherein the pH adjuster comprises a pharmaceutically acceptable acid or base.

85. The method of claim 78, wherein the chelating agent comprises ethylenediaminetetraacetic acid (EDTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), polyphosphates, porphine, and/or a pharmaceutically acceptable salt thereof.

86. The method of claim 78, further comprising a preservative.

87. The method of claim 78, wherein the carrier comprises:
(a) about 2.5% to about 3% by weight of the carrier of naftifine hydrochloride;
(b) about 18% to about 22% by weight of the carrier of the C₁₋₆ alkyl alcohol;
(c) about 17% to about 23% by weight of the carrier of the alkylene glycol;
(d) about 1.5% to about 2% by weight of the carrier of the polymer;
(e) about 0.5% to about 1.5% by weight of the carrier of the long chain fatty alcohol;
(f) the pH adjuster;
(g) about 0.01% to about 0.3% by weight of the carrier of the chelating agent; and
(h) water; and
wherein the propellant is present at about 5% to about 10% by weight of the formulation.

88. The method of claim 78, wherein the carrier comprises:
(a) about 2.5% to about 3% by weight of the carrier of naftifine hydrochloride;
(b) about 28% to about 32% by weight of the carrier of the C₁₋₆ alkyl alcohol;
(c) about 25% to about 30% by weight of the carrier of the alkylene glycol;
(d) about 1.5% to about 2% by weight of the carrier of the polymer;
(e) about 1% to about 2% by weight of the carrier of the long chain fatty alcohol;
(f) the pH adjuster;
(g) about 0.01% to about 0.3% by weight of the carrier of the chelating agent; and
(h) water; and
wherein the propellant is present at about 5% to about 10% by weight of the formulation.

89. The method of claim 87, wherein the carrier comprises:
(a) about 2.5% to about 3% by weight of the carrier of naftifine hydrochloride;
(b) about 20% by weight of the carrier of ethanol;
(c) about 19% by weight of the carrier of propylene glycol;
(d) about 1.75% by weight of the carrier of hydroxypropyl cellulose;
(e) about 1% by weight of the carrier of cetostearyl alcohol;
(f) about 0.14% to about 0.17% by weight of the carrier of trolamine;
(g) about 0.02% by weight of the carrier of disodium edetate; and
(h) water; and
wherein the propellant is AP-70 propellant at about 8% by weight of the formulation.

90. The method of claim 88, wherein the carrier comprises:
(a) about 2.5% to about 3% by weight of the carrier of naftifine hydrochloride;
(b) about 30% by weight of the carrier of ethanol;
(c) about 28.5% by weight of the carrier of propylene glycol;
(d) about 1.75% by weight of the carrier of hydroxypropyl cellulose;
(e) about 1.5% by weight of the carrier of cetostearyl alcohol;
(f) about 0.14% to about 0.17% by weight of the carrier of trolamine;
(g) about 0.02% by weight of the carrier of disodium edetate; and
(h) water; and
wherein the propellant is AP-70 propellant at about 8% by weight of the formulation.

91. The method of claim 78, wherein:
(1) the percentage of naftifine released from the foam within 30 minutes of application is at least about 75% to at least about 90%; and/or
(2) the percentage of naftifine released from the foam within 3 hours of application is at least about 95% to at least about 99%.

92. The method of claim 78, wherein:
(1) a release rate of naftifine from the foam is higher than a release rate of naftifine from a comparable gel or cream formulation; and/or
(2) a maximal release of naftifine from the foam occurs earlier than a comparable gel or cream formulation.

93. The method of claim 78, wherein the foam is more effective in eliminating fungal infection than a placebo treatment or no treatment.

94. The method of claim 93, wherein the effectiveness of the foam is assessed by:
(1) comparing a clinical score of a subject treated with the foam to that of a subject treated with a placebo treatment or given no treatment; and/or
(2) comparing a number of mycologically affected hairs obtained from a subject treated with the foam and that of mycologically affected hairs obtained from a subject treated with a placebo or given no treatment.

95. The method of claim 78, wherein the tinea capitis is associated with Trichophyton mentagrophytes, Microsporum canis, and/or Trichophyton tonsurans.

96. The method of claim 78, wherein the foam is applied for at least 2 to 7 consecutive days. (New) A foamable formulation comprising:
(1) a carrier comprising:
(a) about 2% to about 4% by weight of the carrier of naftifine or a pharmaceutically acceptable salt thereof;
(b) about 15% to about 35% by weight of the carrier of a C₁₋₆ alkyl alcohol;
(c) about 15% to about 30% by weight of the carrier of an alkylene glycol;
(d) about 1% to about 2% by weight of the carrier of a polymer;
(e) about 0.5% to about 2% by weight of the carrier of a long chain fatty alcohol;
(f) a pH adjuster;
(g) about 0.01% to about 0.3% by weight of the carrier of a chelating agent; and
(h) water; and
(2) a liquefied or compressed gas propellant, wherein the propellant is present at about 5% to about 15% by weight of the formulation.
